(19) United States
(12) Patent Application Publication
(10) Pub. No.: US 2025/0332249 A1
(43) Pub. Date: Oct. 30, 2025

(54) CYTOMEGALOVIRUS T CELL EPITOPES AND USES THEREOF

(71) Applicant: LA JOLLA INSTITUTE FOR IMMUNOLOGY, La Jolla, CA (US)

(72) Inventors: Christopher BENEDICT, La Jolla, CA (US); Alessandro SETTE, La Jolla, CA (US)

(21) Appl. No.: 18/560,626

(22) PCT Filed: May 13, 2022
(86) PCT No.: PCT/IB2022/054452

§ 371 Date: Nov. 13, 2023

Related U.S. Application Data
(60) Provisional application No. 63/187,933, filed on May 13, 2021.

(51) Int. Cl. A61K 39/245; A61K 39/00; A61P 31/22; C07K 14/005; C12N 7/00; G01N 33/569

(52) U.S. Cl. CPC A61K 39/245; A61P 31/22; C07K 14/005; C12N 7/00; G01N 33/56972; G01N 33/56994; A61K 2039/572; C12N 2710/16122; C12N 2710/16134; G01N 2333/045

(57) ABSTRACT

The present invention includes compositions and methods for detecting the presence of: a cytomegalovirus or an immune response relevant to a cytomegalovirus infection including T cells responsive to one or more cytomegalovirus peptides or proteins comprising, consisting of, or consisting essentially of: one or more amino acid sequences selected from those sequences set forth in Table 1 or Table 2, or a subsequence, portion, homologue, variant or derivative thereof; a fusion protein comprising one or more amino acid sequences selected from those sequences set forth in Table 1 or Table 2; a pool of 2 or more peptides selected from the amino acid sequences set forth in Table 1 or Table 2; or a polynucleotide that encodes one or more peptides or proteins, comprising, consisting of, or consisting essentially of an amino acid sequence selected from those sequences set forth in Table 1 or Table 2, or a subsequence, portion, homologue, variant or derivative thereof. The invention further provides vaccines, diagnostics, therapies, and kits, comprising such proteins or peptides.

### CROSS-REFERENCE TO RELATED APPLICATIONS

This application is a Section 371 National Stage Application of International Application PCT/IB2022/054452, filed on May 13, 2022, entitled “CYTOMEGALOVIRUS T CELL EPITOPES AND USES THEREOF”, which claims priority to U.S. Provisional Patent Application No. 63/187,933, titled “CYTOMEGALOVIRUS T CELL EPITOPES AND USES THEREOF,” which was filed on May 13, 2021. The entire contents of the foregoing patent applications are incorporated herein by reference, including all text, tables and drawings.

### STATEMENT OF FEDERALLY FUNDED RESEARCH

This invention was made with government support under R01 AI139749 to the La Jolla Institute for Immunology awarded by the National Institutes of Health (NIH). The government has certain rights in the invention.

### SEQUENCE LISTING

The instant application contains a Sequence Listing which has been submitted electronically in ASCII format and is hereby incorporated by reference in its entirety. Said ASCII copy, created on Oct. 2, 2023, is named “051501-0577228_Sequence_Listing” and is 91,662 bytes in size.

### TECHNICAL FIELD OF THE INVENTION

The present invention relates in general to the field of peptides that are T cell epitopes for human cytomegalovirus (hCMV), and more particularly, to compositions and methods for the prevention, treatment, diagnosis, kits comprising, and uses of such T cell epitopes.

### BACKGROUND OF THE INVENTION

Without limiting the scope of the invention, its background is described in connection with human Cytomegalovirus (hCMV).

Cytomegalovirus is a significant human pathogen. It is the number one infectious cause of congenital birth defects, is strongly associated with vascular disease and can cause serious disease in immune compromised patients.

A need remains for better processes, compositions, and methods for screening hCMV positive patients, isolating hCMV epitope-specific immune cells for use in vaccine design and proof of efficacy, and further characterization of hCMV immune cell phenotypes and effector functions, in particular, CD4 T cell epitopes.

### SUMMARY OF THE INVENTION

In one embodiment, the present invention includes a composition comprising: one or more peptides or proteins, comprising, consisting of, or consisting essentially of an amino acid sequence selected from the sequences set forth in Table 1 or Table 2, or a subsequence, portion, homologue, variant or derivative thereof; a fusion protein comprising one or more amino acid sequences selected from those sequences set forth in Table 1 or Table 2; a pool of 2 or more or more peptides comprising, consisting of, or consisting essentially of amino acid sequences selected from those sequences set forth in Table 1 or Table 2; or a polynucleotide that encodes one or more peptides or proteins, comprising, consisting of, or consisting essentially of an amino acid sequence selected from those sequences set forth in Table 1 or Table 2, or a subsequence, portion, homologue, variant or derivative thereof. In one aspect, the one or more peptides or proteins comprises, or wherein the fusion protein comprises 2 or more or more amino acid sequences selected from those sequences set forth in Table 1 or Table 2, or a subsequence, portion, homologue, variant or derivative thereof. In another aspect, the amino acid sequence is selected from a cytomegalovirus T cell epitope selected from those sequences set forth in Table 1 or Table 2 In another aspect, the composition comprises one or more HCMV peptides amino acid sequences selected from those sequences set forth in Table 1 or Table 2, or a subsequence, portion, homologue, variant or derivative thereof; a fusion protein comprising one or more amino acid sequences selected from those sequences set forth in Table 1 or Table 2; or a pool of 2 or more peptides selected from those sequences set forth in Table 1 or Table 2. In another aspect, the peptide or protein comprises a cytomegalovirus T cell epitope. In another aspect, the one or more peptides or proteins comprises a cytomegalovirus CD8+ or CD4+ T cell epitope. In another aspect, the cytomegalovirus is HCMV and the HCMV T cell epitope is not conserved in another cytomegalovirus. In another aspect, the cytomegalovirus is HCMV and the HCMV T cell epitope is conserved in another cytomegalovirus. In another aspect, the one or more peptides or proteins has a length from about 9-15, 15-20, 20-25, 25-30, 30-40, 40-50, 50-75 or 75-100 amino acids. In another aspect, the one or more peptides or proteins elicits, stimulates, induces, promotes, increases or enhances a T cell response to a cytomegalovirus. In another aspect, the one or more peptides or proteins that elicits, stimulates, induces, promotes, increases or enhances the T cell response to the cytomegalovirus is a HCMV Glycoprotein B, 65 kDa lower matrix phosphoprotein, HCMVUL83, phosphorylated matrix protein (pp65), tegument protein pp65, 55 kDa immediate-early protein 1, regulatory protein IE1, UL123; IE1, 45 kDa immediate-early protein 2, single-stranded DNA-binding protein, envelope glycoprotein H, glycoprotein H precursor, major capsid protein, or HCMV UL75 protein or peptide, or a variant, homologue, derivative or subsequence thereof.

In another aspect, the composition further comprises formulating the one or more peptides or proteins into an immunogenic formulation with an adjuvant. In another aspect, the adjuvant is selected from the group consisting of adjuvant is selected from the group consisting of alum, aluminum hydroxide, aluminum phosphate, calcium phosphate hydroxide, cytosine-guanosine oligonucleotide (CpG-ODN) sequence, granulocyte macrophage colony stimulating factor (GM-CSF), monophosphoryl lipid A (MPL), poly(I:C), MF59, Quil A, N-acetyl muramyl-L-alanyl-D-isoglutamine (MDP), FIA, montanide, poly (DL-lactide-coglycolide), squalene, virosome, AS03, ASO4, IL-1, IL-2, IL-3, IL-4, IL-5, IL-6, IL-7, IL-8, IL-10, IL-12, IL-15, IL-17, IL-18, STING, CD40L, pathogen-associated molecular patterns (PAMPs), damage-associated molecular pattern molecules (DAMPs), Freund's complete adjuvant, Freund's incomplete adjuvant, transforming growth factor (TGF)-beta antibody or antagonists, A2aR antagonists, lipopolysaccharides (LPS), Fas ligand, Trail, lymphotactin, Mannan (M-FP), APG-2, Hsp70 and Hsp90, pattern recognition receptor ligands, TLR3 ligands, TLR4 ligands, TLR5 ligands, TLR7/8 ligands, and TLR9 ligands.

In another embodiment, the present invention includes a composition comprising monomers or multimers of: peptides or proteins comprising, consisting of, or consisting essentially of: one or more amino acid sequences selected from those sequences set forth in Table 1 or Table 2, concatemers, subsequences, portions, homologues, variants or derivatives thereof; a fusion protein comprising one or more amino acid sequences selected from those sequences set forth in Table 1 or Table 2; or a polynucleotide that encodes one or more peptides or proteins, comprising, consisting of, or consisting essentially of an amino acid sequence selected from those sequences set forth in Table 1 or Table 2, or a subsequence, portion, homologue, variant or derivative thereof.

In another embodiment, the present invention includes a composition comprising one or more peptide-major histocompatibility complex (MHC) monomers or multimers, wherein the peptide-MHC monomer or multimer comprises a peptide comprising, consisting of, or consisting essentially of an amino acid sequence selected from those sequences set forth in Table 1 or Table 2, in a groove of the MHC monomer or multimer.

In another embodiment, the present invention includes a composition comprising: one or more peptides or proteins comprising, consisting of, or consisting essentially of an amino acid sequence selected from those sequences set forth in Table 1 or Table 2, or a subsequence, portion, homologue, variant or derivative thereof; a fusion protein comprising one or more amino acid sequences selected from those sequences set forth in Table 1 or Table 2; a pool of 2 or more peptides selected from those sequences set forth in Table 1 or Table 2; a polynucleotide that encodes one or more peptides or proteins, comprising, consisting of, or consisting essentially of an amino acid sequence selected from those sequences set forth in Table 1 or Table 2, or a subsequence, portion, homologue, variant or derivative thereof. In one aspect, the one or more peptides or proteins comprises, or wherein the fusion protein comprises, 2 or more amino acid sequences selected from those sequences set forth in Table 1 or Table 2, or a subsequence, portion, homologue, variant or derivative thereof. In another aspect, the protein or peptide comprises a HCMV T cell epitope. In another aspect, the one or more peptides or proteins comprises a HCMV CD8+ or CD4+ T cell epitope. In another aspect, the HCMV T cell epitope is not conserved in another cytomegalovirus. In another aspect, the HCMV T cell epitope is conserved in another cytomegalovirus. In another aspect, the one or more peptides or proteins has a length from about 9-15, 15-20, 20-25, 25-30, 30-40, 40-50, 50-75 or 75-100 amino acids. In another aspect, the one or more peptides or proteins elicits, stimulates, induces, promotes, increases or enhances a T cell response to HCMV. In another aspect, the one or more peptides or proteins that elicits, stimulates, induces, promotes, increases or enhances the T cell response to HCMV is an HCMV Glycoprotein B, 65 kDa lower matrix phosphoprotein, HCMVUL83, phosphorylated matrix protein (pp65), tegument protein pp65, 55 kDa immediate-early protein 1, regulatory protein IE1, UL123; IE1, 45 kDa immediate-early protein 2, single-stranded DNA-binding protein, envelope glycoprotein H, glycoprotein H precursor, major capsid protein, or HCMV UL75 protein or peptide, or a variant, homologue, derivative or subsequence thereof. In another aspect, the composition further comprises formulating the one or more peptides or proteins into an immunogenic formulation with an adjuvant. In another aspect, the adjuvant is selected from the group consisting of adjuvant is selected from the group consisting of alum, aluminum hydroxide, aluminum phosphate, calcium phosphate hydroxide, cytosine-guanosine oligonucleotide (CpG-ODN) sequence, granulocyte macrophage colony stimulating factor (GM-CSF), monophosphoryl lipid A (MPL), poly(I.C), MF59, Quil A, N-acetyl muramyl-L-alanyl-D-isoglutamine (MDP), FIA, montanide, poly (DL-lactide-coglycolide), squalene, virosome, ASO3, ASO4, IL-1, IL-2, IL-3, IL-4, IL-5, IL-6, IL-7, IL-8, IL-10, IL-12, IL-15, IL-17, IL-18, STING, CD40L, pathogen-associated molecular patterns (PAMPs), damage-associated molecular pattern molecules (DAMPs), Freund's complete adjuvant, Freund's incomplete adjuvant, transforming growth factor (TGF)-beta antibody or antagonists, A2aR antagonists, lipopolysaccharides (LPS), Fas ligand, Trail, lymphotactin, Mannan (M-FP), APG-2, Hsp70 and Hsp90, pattern recognition receptor ligands, TLR3 ligands, TLR4 ligands, TLR5 ligands, TLR7/8 ligands, and TLR9 ligands.

In another embodiment, the present invention includes a composition comprising monomers or multimers of: one or more peptides or proteins comprising, consisting of, or consisting essentially of: one or more HCMV amino acid sequences selected from those sequences set forth in Table 1 or Table 2, concatemers, subsequences, portions, homologues, variants or derivatives thereof; a fusion protein comprising one or more amino acid sequences selected from those sequences set forth in Table 1 or Table 2; or a polynucleotide that encodes one or more peptides or proteins, comprising, consisting of, or consisting essentially of an amino acid sequence selected from those sequences set forth in Table 1 or Table 2, or a subsequence, portion, homologue, variant or derivative thereof.

In another embodiment, the present invention includes a composition comprising one or more peptide-major histocompatibility complex (MHC) monomers or multimers, wherein the peptide-MHC monomer or multimer comprises a peptide comprising, consisting of, or consisting essentially of an amino acid sequence selected from those sequences set forth in Table 1 or Table 2, in a groove of the (MHC) monomer or multimer.

In another embodiment, the present invention includes a method for detecting the presence of: (i) a cytomegalovirus or (ii) an immune response relevant to cytomegalovirus infections, vaccines or therapies, including T cells responsive to one or more cytomegalovirus peptides, comprising: providing one or more proteins or peptides for detection of an amount or a relative amount of, and/or the activity of, and/or the state of antigen-specific T-cells; contacting a biological sample suspected of having cytomegalovirus-specific T-cells to one or more proteins or peptides for detection; and detecting an amount or a relative amount of, and/or the activity of, and/or the state of antigen-specific T-cells in the biological sample, wherein the one or more proteins or peptides for detection comprise one or more amino acid sequences set forth in Table 1 or Table 2, or comprise a pool of 2 or more or more amino acid sequences set forth in Table 1 or Table 2. In one aspect, detecting the amount or a relative amount of, and/or activity of antigen-specific T-cells comprises one or more steps of identification or detection of the antigen-specific T-cells and measuring the amount of the antigen-specific T-cells. In another aspect, the one or more peptides or proteins comprises 2 or more amino acid sequences selected from Table 1 or Table 2 In another aspect, the detecting the amount or a relative amount of, and/or activity of antigen-specific T-cells comprises indirect detection and/or direct detection. In another aspect, the method of detecting an immune response relevant to the cytomegalovirus comprises the following steps: providing an MHC monomer or an MHC multimer; contacting a population T-cells to the MHC monomer or MHC multimer; and measuring the number, activity or state of T-cells specific for the MHC monomer or MHC multimer. In one aspect, the MHC monomer or MHC multimer comprises a protein or peptide of the cytomegalovirus. In another aspect, the protein or peptide comprises a CD8+ or CD4+ T cell epitope. In another aspect, the T cell epitope is not conserved in another cytomegalovirus. In another aspect, the T cell epitope is conserved in another cytomegalovirus. In another aspect, the protein or peptide has a length from about 9-15, 15-20, 20-25, 25-30, 30-40, 40-50, 50-75 or 75-100 amino acids. In another aspect, the proteins or peptides comprise 2 or more amino acid sequences selected from those sequences set forth in Table 1 or Table 2, or a subsequence, portion, homologue, variant or derivative thereof. In another aspect, the method further comprises detecting the presence or amount of the one or more peptides in a biological sample, or a response thereto, which is diagnostic of a cytomegalovirus infection. In another aspect, the detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay. In another aspect, the method further comprises administering a treatment comprising the composition of one or more proteins, peptides or multimers to the subject from which the biological sample was drawn that increases the amount or relative amount of, and/or activity of the antigen-specific T-cells.

In another embodiment, the present invention includes a method for detecting the presence of: (i) HCMV or (ii) an immune response relevant to HCMV infections, vaccines or therapies, including T cells responsive to one or more HCMV peptides, comprising: providing one or more proteins or peptides for detection of an amount or a relative amount of, and/or the activity of, and/or the state of antigen-specific T-cells; contacting a biological sample suspected of having HCMV-specific T-cells to one or more proteins or peptides for detection; and detecting an amount or a relative amount of, and/or the activity of, and/or the state of antigen-specific T-cells in the biological sample, wherein the one or more proteins or peptides for detection comprise one or more amino acid sequences set forth in those sequences set forth in Table 1 or Table 2, or comprise a pool of 2 or more amino acid sequences set forth in those sequences set forth in Table 1 or Table 2. In one aspect, detecting the amount or a relative amount of, and/or activity of antigen-specific T-cells comprises one or more steps of identification or detection of the antigen-specific T-cells and measuring the amount of the antigen-specific T-cells. In another aspect, the one or more peptides or proteins comprises 2 or more amino acid sequences selected from those sequences set forth in Table 1 or Table 2. In another aspect, detecting the amount or a relative amount of, and/or activity of antigen-specific T-cells comprises indirect detection and/or direct detection. In another aspect, detecting an immune response relevant to HCMV comprises the following steps: providing an MHC monomer or an MHC multimer; contacting a population T-cells to the MHC monomer or MHC multimer; and measuring the number, activity or state of T-cells specific for the MHC monomer or MHC multimer. In another aspect, the MHC monomer or MHC multimer comprises a protein or peptide of HCMV. In another aspect, the protein or peptide comprises a HCMV CD8+ or CD4+ T cell epitope. In another aspect, the HCMV T cell epitope is not conserved in another cytomegalovirus. In another aspect, the HCMV T cell epitope is conserved in another cytomegalovirus. In another aspect, the protein or peptide has a length from about 9-15, 15-20, 20-25, 25-30, 30-40, 40-50, 50-75 or 75-100 amino acids. In another aspect, the proteins or peptides comprise 2 or more amino acid sequences selected from those sequences set forth in Table 1 or Table 2, or a subsequence, portion, homologue, variant or derivative thereof. In another aspect, the method further comprises detecting the presence or amount of the one or more peptides in a biological sample, or a response thereto, which is diagnostic of a HCMV infection. In another aspect, detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay. In another aspect, the method further comprises administering a treatment comprising the composition of one or more proteins, peptides or multimers to the subject from which the biological sample was drawn that increases the amount or relative amount of, and/or activity of the antigen-specific T-cells.

In another embodiment, the present invention includes a method detecting a cytomegalovirus infection or exposure in a subject, the method comprising, consisting of, or consisting essentially of: contacting a biological sample from a subject with a composition of composition of one or more proteins, peptides or multimers; and determining if the composition elicits an immune response from the contacted cells, wherein the presence of an immune response indicates that the subject has been exposed to or infected with cytomegalovirus. In one aspect, the sample comprises T cells. In another aspect, the response comprises inducing, increasing, promoting or stimulating anti-cytomegalovirus activity of T cells. In another aspect, the T cells are CD8+ or CD4+ T cells. In another aspect, the method comprises determining whether the subject has been infected by or exposed to the cytomegalovirus more than once by determining if the subject elicits a secondary T cell immune response profile that is different from a primary T cell immune response profile. In another aspect, the method further comprises diagnosing a cytomegalovirus infection or exposure in a subject, the method comprising contacting a biological sample from a subject with a composition of composition of one or more proteins, peptides or multimers, and determining if the composition elicits a T cell immune response, wherein the T cell immune response identifies that the subject has been infected with or exposed to a cytomegalovirus. In another aspect, the method is conducted three or more days following the date of suspected infection by or exposure to a cytomegalovirus.

In another embodiment, the present invention includes a method detecting HCMV infection or exposure in a subject, the method comprising, consisting of, or consisting essentially of: contacting a biological sample from a subject with a composition of composition of one or more proteins, peptides or multimers; and determining if the composition elicits an immune response from the contacted cells, wherein the presence of an immune response indicates that the subject has been exposed to or infected with HCMV. In another aspect, the sample comprises T cells. In another aspect, the response comprises inducing, increasing, promoting or stimulating anti-HCMV activity of T cells. In another aspect, the T cells are CD8+ or CD4+ T cells. In another aspect, the method comprises determining whether the subject has been infected by or exposed to HCMV more than once by determining if the subject elicits a secondary T cell immune response profile that is different from a primary T cell immune response profile. In another aspect, the method further comprises diagnosing a HCMV infection or exposure in a subject, the method comprising contacting a biological sample from a subject with a composition of one or more proteins, peptides or multimers; and determining if the composition elicits a T cell immune response, wherein the T cell immune response identifies that the subject has been infected with or exposed to HCMV. In another aspect, the method is conducted three or more days following the date of suspected infection by or exposure to a cytomegalovirus.

In another embodiment, the present invention includes a kit for the detection of cytomegalovirus or an immune response to cytomegalovirus in a subject comprising, consisting of or consisting essentially of: one or more T cells that specifically detect the presence of: one or more amino acid sequences selected from those sequences set forth in Table 1 or Table 2, or a subsequence, portion, homologue, variant or derivative thereof; or a fusion protein comprising one or more amino acid sequences selected from those sequences set forth in Table 1 or Table 2; or a pool of 2 or more or more peptides selected from the amino acid sequences set forth in Table 1 or Table 2. In one aspect, the one or more amino acid sequences are selected from a cytomegalovirus T cell epitope set forth in Table 1 or Table 2. In another aspect, the composition comprises: one or more amino acid sequences selected from those sequences set forth in Table 1 or Table 2, or a subsequence, portion, homologue, variant or derivative thereof; a fusion protein comprising one or more amino acid sequences selected from those sequences set forth in Table 1 or Table 2; or a pool of 2 or more peptides selected from the amino acid sequences set forth in those sequences set forth in Table 1 or Table 2. In another aspect, the amino acid sequence comprises a cytomegalovirus CD8+ or CD4+ T cell epitope. In another aspect, the T cell epitope is not conserved in another cytomegalovirus. In another aspect, the T cell epitope is conserved in another cytomegalovirus. In another aspect, the fusion protein has a length from about 9-15, 15-20, 20-25, 25-30, 30-40, 40-50, 50-75 or 75-100 amino acids. In another aspect, the kit includes instruction for a diagnostic method, a process, a composition, a product, a service or component part thereof for the detection of: (i) cytomegalovirus or (ii) an immune response relevant to cytomegalovirus infections, vaccines or therapies, including T cells responsive to cytomegalovirus. In another aspect, the kit includes reagents for detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay. In another aspect, the kit includes reagents for determining a Human Leukocyte Antigen (HLA) profile of a subject, and selecting peptides that are presented by the HLA profile of the subject for detecting an immune response to cytomegalovirus.

In another embodiment, the present invention includes a kit for the detection of HCMV or an immune response to HCMV in a subject comprising, consisting of or consisting essentially of: one or more T cells that specifically detect the presence of: one or more amino acid sequences selected from those sequences set forth in Table 1 or Table 2, or a subsequence, portion, homologue, variant or derivative thereof; a fusion protein comprising one or more amino acid sequences selected from those sequences set forth in Table 1 or Table 2; or a pool of 2 or more peptides selected from the amino acid sequences set forth in those sequences set forth in Table 1 or Table 2. In another aspect, the amino acid sequence comprises a HCMV CD8+ or CD4+ T cell epitope. In another aspect, the HCMV T cell epitope is not conserved in another cytomegalovirus. In another aspect, the HCMV T cell epitope is conserved in another cytomegalovirus. In another aspect, the fusion protein has a length from about 9-15, 15-20, 20-25, 25-30, 30-40, 40-50, 50-75 or 75-100 amino acids. In another aspect, the kit includes instruction for a diagnostic method, a process, a composition, a product, a service or component part thereof for the detection of: (i) HCMV or (ii) an immune response relevant to HCMV infections, vaccines or therapies, including T cells responsive to HCMV. In another aspect, the kit includes reagents for detecting an amount or a relative amount of, and/or the activity of, and/or the state of antigen-specific T-cells in the biological sample comprises measuring one or more of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, or a cytokine proliferation assay. In another aspect, the kit includes reagents for determining a Human Leukocyte Antigen (HLA) profile of a subject, and selecting peptides that are presented by the HLA profile of the subject for detecting an immune response to HCMV.

In another embodiment, the present invention includes a method of stimulating, inducing, promoting, increasing, or enhancing an immune response against a cytomegalovirus in a subject, comprising: administering a composition of one or more proteins, peptides, multimers or a polynucleotide that expresses the protein, peptide or multimers, in an amount sufficient to stimulate, induce, promote, increase, or enhance an immune response against the cytomegalovirus in the subject. In another aspect, the immune response provides the subject with protection against a cytomegalovirus infection or pathology, or one or more physiological conditions, disorders, illnesses, diseases or symptoms caused by or associated with cytomegalovirus infection or pathology. In another aspect, the immune response is specific to: one or more HCMV peptides selected from the amino acid sequences set forth in Table 1 or Table 2, or a subsequence, portion, homologue, variant or derivative thereof.

In another embodiment, the present invention includes a method of stimulating, inducing, promoting, increasing, or enhancing an immune response against HCMV in a subject, comprising: administering a composition of proteins, peptides, multimers or a polynucleotide that expresses the protein, peptide or multimers, in an amount sufficient to stimulate, induce, promote, increase, or enhance an immune response against HCMV in the subject. In one aspect, the immune response provides the subject with protection against a HCMV infection or pathology, or one or more physiological conditions, disorders, illnesses, diseases or symptoms caused by or associated with HCMV infection or pathology. In another aspect, the immune response is specific to: one or more HCMV peptides selected from the amino acid sequences set forth in those sequences set forth in Table 1 or Table 2, or a subsequence, portion, homologue, variant or derivative thereof.

In another embodiment, the present invention includes a method of stimulating, inducing, promoting, increasing, or enhancing an immune response against HCMV in a subject, comprising: administering to a subject an amount of a protein or peptide comprising, consisting of or consisting essentially of an amino acid sequence of the HCMV Glycoprotein B, 65 kDa lower matrix phosphoprotein, HCMV UL83, phosphorylated matrix protein (pp65), tegument protein pp65, 55 kDa immediate-early protein 1, regulatory protein IE1, UL123; IE1, 45 kDa immediate-early protein 2, single-stranded DNA-binding protein, envelope glycoprotein H, glycoprotein H precursor, major capsid protein, or HCMV UL75 protein or peptide, or a variant, homologue, derivative or subsequence thereof, wherein the protein or peptide comprises at least two peptides selected from the amino acid sequences set forth in Table 1 or Table 2 or a subsequence, portion, homologue, variant or derivative thereof, in an amount sufficient to prevent, stimulate, induce, promote, increase, immunize against, or enhance an immune response against HCMV in the subject. In one aspect, the immune response provides the subject with protection against HCMV infection or pathology, or one or more physiological conditions, disorders, illnesses, diseases or symptoms caused by or associated with HCMV infection or pathology.

In another embodiment, the present invention includes a method of treating, preventing, or immunizing a subject against HCMV infection, comprising administering to a subject an amount of a protein or peptide comprising, consisting of, or consisting essentially of an amino acid sequence of a cytomegalovirus HCMV Glycoprotein B, 65 kDa lower matrix phosphoprotein, HCMV UL83, phosphorylated matrix protein (pp65), tegument protein pp65, 55 kDa immediate-early protein 1, regulatory protein IE1, UL123; IE1, 45 kDa immediate-early protein 2, single-stranded DNA-binding protein, envelope glycoprotein H, glycoprotein H precursor, major capsid protein, or HCMV UL75 protein or peptide, or a variant, homologue, derivative or subsequence thereof, wherein the protein or peptide comprises at least two amino acid sequences selected from Table 1 or Table 2 or a subsequence, portion, homologue, variant or derivative thereof, in an amount sufficient to treat, prevent, or immunize the subject for HCMV infection, wherein the protein or peptide comprises or consists of a cytomegalovirus T cell epitope that elicits, stimulates, induces, promotes, increases, or enhances an anti-HCMV T cell immune response. In one aspect, the one or more amino acid sequences are selected from those sequences set forth in Table 1 or Table 2, or a subsequence, portion, homologue, variant or derivative thereof; a fusion protein comprising one or more amino acid sequences selected from those sequences set forth in Table 1 or Table 2; or a pool of 2 or more peptides selected from the amino acid sequences set forth in those sequences set forth in Table 1 or Table 2. In one aspect, the anti-HCMV T cell response is a CD8+, a CD4+ T cell response, or both. In another aspect, the T cell epitope is conserved across two or more clinical isolates of HCMV, two or more circulating forms of HCMV, or two or more cytomegaloviruses. In another aspect, the HCMV infection is an acute infection. In another aspect, the subject is a mammal or a human. In another aspect, the method reduces HCMV viral titer, increases or stimulates HCMV viral clearance, reduces or inhibits HCMV viral proliferation, reduces or inhibits increases in HCMV viral titer or HCMV viral proliferation, reduces the amount of a HCMV viral protein or the amount of a HCMV viral nucleic acid, or reduces or inhibits synthesis of a HCMV viral protein or a HCMV viral nucleic acid. In another aspect, the method reduces one or more adverse physiological conditions, disorders, illness, diseases, symptoms or complications caused by or associated with HCMV infection or pathology. In another aspect, the method improves or prevents one or more adverse physiological conditions, disorders, illness, diseases, symptoms or complications caused by or associated with HCMV infection or pathology, for example, pneumonia, hepatitis, encephalitis, jaundice, etc. In another aspect, the symptom is fever or chills, perspiration, cough, fatigue, uneasiness, sore throat, swollen glands, joint and muscle pain, low appetite, weight loss, diarrhea, ulcerations in the mouth and/or gastrointestinal system, gastrointestinal bleeding, shortness of breath, hypoxemia, problems with vision (blind spots, blurred vision, etc.), inflamed liver, inflammation of the brain, rash, and/or skin spots or splotches. In another aspect, the method reduces or inhibits susceptibility to HCMV infection or pathology. In another aspect, the protein or peptide, or a subsequence, portion, homologue, variant or derivative thereof, is administered prior to, substantially contemporaneously with or following exposure to or infection of the subject with HCMV. In another aspect, a plurality of HCMV T cell epitopes are administered prior to, substantially contemporaneously with or following exposure to or infection of the subject with HCMV. In another aspect, the protein or peptide, or a subsequence, portion, homologue, variant or derivative thereof is administered within 2-72 hours, 2-48 hours, 4-24 hours, 4-18 hours, or 6-12 hours after a symptom of HCMV infection or exposure develops. In another aspect, the protein or peptide, or a subsequence, portion, homologue, variant or derivative thereof is administered prior to exposure to or infection of the subject with HCMV.

In another embodiment, the present invention includes a method of treating, preventing, or immunizing a subject against HCMV infection, comprising administering to a subject the composition of one or more proteins, peptides or multimers in an amount sufficient to treat, prevent, or immunize the subject for HCMV infection. In one aspect, the HCMV infection is an acute infection. In another aspect, the method reduces HCMV viral titer, increases or stimulates HCMV viral clearance, reduces or inhibits HCMV viral proliferation, reduces or inhibits increases in HCMV viral titer or HCMV viral proliferation, reduces the amount of a HCMV viral protein or the amount of a HCMV viral nucleic acid, or reduces or inhibits synthesis of a HCMV viral protein or a HCMV viral nucleic acid. In another aspect, the method reduces one or more adverse physiological conditions, disorders, illness, diseases, symptoms or complications caused by or associated with HCMV infection or pathology. In another aspect, the method improves or prevents one or more adverse physiological conditions, disorders, illness, diseases, symptoms or complications caused by or associated with HCMV infection or pathology, for example, pneumonia, hepatitis, encephalitis, jaundice, etc. In another aspect, the symptom is fever or chills, perspiration, cough, fatigue, uneasiness, sore throat, swollen glands, joint and muscle pain, low appetite, weight loss, diarrhea, ulcerations in the mouth and/or gastrointestinal system, gastrointestinal bleeding, shortness of breath, hypoxemia, problems with vision (blind spots, blurred vision, etc.), inflamed liver, inflammation of the brain, rash, and/or skin spots or splotches. In another aspect, the method reduces or inhibits susceptibility to HCMV infection or pathology. In another aspect, the composition is administered prior to, substantially contemporaneously with or following exposure to or infection of the subject with HCMV. In another aspect, the composition is administered prior to, substantially contemporaneously with or following exposure to or infection of the subject with HCMV. In another aspect, the composition is administered within 2-72 hours, 2-48 hours, 4-24 hours, 4-18 hours, or 6-12 hours after a symptom of HCMV infection or exposure develops. In another aspect, the composition is administered prior to exposure to or infection of the subject with HCMV.

In another embodiment, the present invention includes a peptide or peptides that are immunoprevalent or immunodominant in a virus obtained by a method consisting of, or consisting essentially of: obtaining an amino acid sequence of the virus; determining one or more sets of overlapping peptides spanning one or more virus antigen using unbiased selection; synthesizing one or more pools of virus peptides comprising the one or more sets of overlapping peptides; combining the one or more pools of virus peptides with Class I major histocompatibility proteins (MHC), Class II MHC, or both Class I and Class II MHC to form peptide-MHC complexes; contacting the peptide-MHC complexes with T cells from subjects exposed to the virus; determining which pools triggered cytokine release by the T cells; and deconvoluting from the pool of peptides that elicited cytokine release by the T cells, which peptide or peptides are immunoprevalent or immunodominant in the pool. In one aspect, the virus is a cytomegalovirus. In another aspect, the cytomegalovirus is HCMV. In another aspect, the immunodominant peptides are selected from 1, 2 or more peptides selected from the amino acid sequences set forth in Table 1 or Table 2. In another aspect, the immunodominant peptides are selected from 1, 2 or more peptides selected from the amino acid sequences set forth in those sequences set forth in Table 1 or Table 2.

In another embodiment, the present invention includes a method of selecting an immunoprevalent or immunodominant peptide or protein of a virus comprising, consisting of, or consisting essentially of: obtaining an amino acid sequence of the virus; determining one or more sets of overlapping peptides spanning one or more virus antigen using unbiased selection; synthesizing one or more pools of virus peptides comprising the one or more sets of overlapping peptides; combining the one or more pools of virus peptides with Class I major histocompatibility proteins (MHC), Class II MHC, or both Class I and Class II MHC to form peptide-MHC complexes; contacting the peptide-MHC complexes with T cells from subjects exposed to the virus; determining which pools triggered cytokine release by the T cells; and deconvoluting from the pool of peptides that elicited cytokine release by the T cells, which peptide or peptides are immunoprevalent or immunodominant in the pool. In one aspect, the virus is a cytomegalovirus. In another aspect, the cytomegalovirus is HCMV. In another aspect, the immunodominant peptides are selected from 1, 2 or more peptides selected from the amino acid sequences set forth in Table 1 or Table 2. In another aspect, the immunodominant peptides are selected from 1, 2 or more peptides selected from the amino acid sequences set forth in those sequences set forth in Table 1 or Table 2.

In another embodiment, the present invention includes a polynucleotide that expresses one or more peptides or proteins, comprising, consisting of, or consisting essentially of an amino acid sequence selected from those sequences set forth in Table 1 or Table 2, or a subsequence, portion, homologue, variant or derivative thereof; a fusion protein comprising one or more amino acid sequences selected from those sequences set forth in Table 1 or Table 2; or a pool of 2 or more or more peptides comprising, consisting of, or consisting essentially of amino acid sequences selected from those sequences set forth in Table 1 or Table 2. In one aspect, the vector comprises the polynucleotide of claim that expresses one or more peptides or proteins, comprising, consisting of, or consisting essentially of an amino acid sequence selected from those sequences set forth in Table 1 or Table 2, or a subsequence, portion, homologue, variant or derivative thereof; a fusion protein comprising one or more amino acid sequences selected from those sequences set forth in Table 1 or Table 2; or a pool of 2 or more or more peptides comprising, consisting of, or consisting essentially of amino acid sequences selected from those sequences set forth in Table 1 or Table 2, a viral vector, or a host cell the comprises the same.

In another embodiment, the present invention includes a polynucleotide that expresses one or more peptides or proteins comprising, consisting of, or consisting essentially of an amino acid sequence selected from those sequences set forth in Table 1 or Table 2, or a subsequence, portion, homologue, variant or derivative thereof; a fusion protein comprising one or more amino acid sequences selected from those sequences set forth in Table 1 or Table 2; or a pool of 2 or more peptides selected from those sequences set forth in Table 1 or Table 2. In one aspect, the vector comprises the polynucleotide of claim that expresses one or more peptides or proteins comprising, consisting of, or consisting essentially of an amino acid sequence selected from those sequences set forth in Table 1 or Table 2, or a subsequence, portion, homologue, variant or derivative thereof; a fusion protein comprising one or more amino acid sequences selected from those sequences set forth in Table 1 or Table 2; or a pool of 2 or more peptides selected from those sequences set forth in Table 1 or Table 2, a viral vector, or a host cell that comprises the same.

### BRIEF DESCRIPTION OF THE DRAWINGS

For a more complete understanding of the features and advantages of the present invention, reference is now made to the detailed description of the invention along with the accompanying figures and in which:

FIG. 1 provides a non-limiting example of a Strategy for CMV specific epitope identification: PBMCs from HCMV seropositive subjects were stimulated with 2 μg/ml pools and plated on IFN-γ coated fluorospot plates for 20 hours. The top 10 positive pools (indicated by * on bars) were deconvoluted to identify individual epitopes. PBMC were stimulated with 10 μg/ml of each individual peptide contained in the pool and reactivity was measured by IFN-γ fluorospot. (A) SFC/10⁶ PBMC for one representative subject against the 89 peptide pools (B) Deconvoluted pool representing individual peptides (C) Intracellular IFN-γ staining representing the CD4+ and CD8+ phenotype of the immune responses.

FIG. 2 provides a non-limiting example of the Breadth, dominance and phenotypic characterization of CMV responses: A. The number of epitopes recognized by each donor, mean±range. B. Proportion of the 19 donors that responded to the indicated number of epitopes. (C and D). The number of epitopes and the % response identified in one, two, three, four or more than equal to five donors. (E and F). The number of events and % response attributed to CD4+ and CD8+ responses of dominant epitopes (n=58) that demonstrated response frequency of 0.15 (15%).

FIG. 3 provides a non-limiting example of Epitope distribution by ORF of origin: 235 epitopes mapped to 89 ORFs. Left Y axis denotes the number of epitopes associated with each ORF (bars) and right Y axis denotes the response frequency associated with each ORFs (dotted line). ORFs inducing both Class I and Class II responses are highlighted in red and ORFs that mount only Class I responses are blue. ORFL147C is the first ‘novel’ ORF identified by rRNA profiling from left-to-right, and only induces responses in 2/19 individuals tested.

FIG. 4 provides a non-limiting example of the overlap between the IEDB and newly identified immunogenic ORFs identified in the present screen. 7 ORFs were shared between IEDB and the present screen, 82 ORFs were novel in terms of the inducing T cell responses. 52 of 82 ORFs were canonical and 30 were identified by recent ribosomal studies.

FIG. 5 provides a non-limiting example of Antigen specific CD4+ T cell responses in CMV (+) and CMV (−) subjects detected with different HCMV peptide pools: (A) Representative FACS plots showing CMV specific CD4+ T cell reactivity against different peptide pools based on activation-induced marker assays (OX40+ and CD137+ double expression). PBMCs from CMV (+) (red circles) and CMV (−) donors (grey circles) were stimulated with 2 μg/ml of the Mabtech pool or IEDB-II/P235 pools for 24 hrs. (B). Epitope-pool specific CD4+ T cells measured as percentage of activation-induced marker assay positive (OX40+CD137+) CD4+ T cells. Each dot represents an individual subject. CMV (+) subjects demonstrated significantly higher CD4 T cell AIM responses than CMV (−) subjects with all the different pools tested. Mabtech CMV+ vs CMV− p=0.0007; P235 CMV+ vs CMV− p=0.0065; IEDB-II CMV+ vs CMV− p=0.0009; P235/IEDB-II CMV+ vs CMV− p=0.004. Two-tailed Mann-Whitney test. Comparisons were made using the Wilcoxon matched-pairs signed ranked test, Two-tailed p values; Geometric mean with geometric standard deviation.

FIG. 6 provides a non-limiting example of the Confirmation of CMV seropositive donors for the T cell screen. (A) IgG levels in plasma of subjects in the screening cohort (n=10 males, n=9 females) and (B) the validation cohort (n=13 males, n=26 females). Determined by ELISA. Dotted line represents the cut off for positivity (10 NTU).

FIG. 7 provides a non-limiting example of the total response captured by the top 10 pools in each subject. The sum of response magnitude mounted by top 10 pools divided by the total response magnitude mounted by all the positive pools in percent. The average % of total response covered by top 10 pools accounted for ˜90% response.

FIG. 8 provides a non-limiting example of the Response magnitude of each epitope identified in CMV seropositive individuals: Each dot represents an epitope. Y axis represents the response magnitude of individual epitopes. X axis represents each subject. Median±interquartile range is shown.

FIG. 9 provides a non-limiting example of the Frequency and magnitude of response in males and females: Each dot represents a donor. Black dot/bar represents males and red dot/bar represents females. Median with interquartile range is displayed. Two-tailed Mann-Whitney test.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention. Numerous variations, changes, and substitutions will now occur to those skilled in the art without departing from the invention. It should be understood that various alternatives to the embodiments of the invention described herein may be employed in practicing the invention. To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims. Unless specifically stated or obvious from context, as used herein, the term “or” is understood to be inclusive.

Despite the prevalence and medical significance of human cytomegalovirus (HCMV) infections, a systematic analysis of the targets of T cell recognition in humans, spanning the entire genome and including recently described potential novel ORFs (1), has not yet been generated. The inventors screened a library of epitopes predicted to bind HLA class II spanning over 500 different HCMV ORFs, including ˜250 previously described and ˜270 recently described potential novel ORFS, using an ex vivo IFNγ fluorospot assay. 235 unique HCMV specific epitopes derived from over 100 ORFs were identified, including previously described immunodominant ones and several additional that were not previously described to be immunogenic. Of those, 29 belong to the set of recently reported novel ORFs, thus providing evidence that at least some of these are actually expressed in vivo in humans. These data reveal that the breadth of the human T cell response to HCMV is much greater than previously thought. The ORFs and epitopes identified help elucidate how T cell immunity relates to HCMV pathogenesis and instruct ongoing HCMV vaccine research.

The inventors have identified greater than two hundred novel peptide epitopes which are targeted by HCMV-specific antiviral T cells. These encompass the immune response against 82 HCMV ORFs where no epitope has previously been described. In certain embodiments, these epitopes can be used in clinical diagnostics for screening HCMV infected people for the magnitude of their virus specific T cell response, especially CD4 T cells. In other embodiments, these epitopes comprise new targets for HCMV vaccine development. In further embodiments, these epitopes are used to isolate HCMV-specific T cells from patients undergoing cellular immunotherapy in cases of CMV-risk in immune ablating procedures (by way of example and not by way of limitation, bone marrow transplantation, kidney transplantation, etc.).

### Example 1

Human cytomegalovirus (HCMV, HHV-5) is a s-herpesvirus that infects the majority of the world's population. Infection in healthy persons is characterized by a primary asymptomatic phase followed by the establishment of lifelong persistence/latency in several cell types (2, 3). HCMV's 236 kbp double stranded DNA genome facilitates its persistence and reactivation when immunity is compromised, with both viral and cellular proteins controlling viral gene expression and regulating the dynamic and reversible latent-lytic cycle that develops over a lifelong infection (4, 5). Although largely persistent, its reactivation in immunocompromised populations, such as transplant recipients and AIDS patients, causes severe disease outcomes (6-12). Congenital infection in the developing fetus is also the leading infectious cause of birth defects (13-19). Moreover, the available antiviral drug therapies are insufficient, and often toxic in young children (20-23). Consequently, HCMV is recognized as a major public health problem, and development of a vaccine that prevents or at least mitigates virus-induced disease is atop priority (24-26).

Although both humoral and cell mediated immune responses protect against HCMV infection, a considerable effort has been made towards identifying HCMV targets of CTL responses due to their pivotal role in controlling HCMV disease in immunocompromised individuals (27-30). However, HCMV targets for CD4+ T helper cell responses that function to amplify CTL and antibody responses, or may mediate direct antiviral activity themselves, remain to be explored in detail. Therefore, it is imperative for the success of an HCMV vaccine to identify and assess the immunogenicity of the large number of candidate viral proteins with the potential to induce robust CD4+ T cell responses.

Previous work from Sylwester et al. extensively characterized the canonical HCMV proteins that are targeted by CD4+ and CD8+ T cell responses (31), and work by many other groups has identified immunodominant epitopes derived from these that include the 65kDA phosphoprotein (UL83/pp65), immediate early protein 1 (UL123), tegument protein pp150 (UL32), envelope glycoprotein B (UL55), viral transcription factor IE2 (UL122), and major capsid protein (UL86) (32-39). However, a comprehensive analysis of HCMV epitope-specific T cell response has been challenging, mainly due to the large size of virus and the evolving impact that persistent infection has on the memory pool. Stern-Ginossar et al. recently reported all HCMV RNAs found to be associated with ribosomes in infected fibroblasts, increasing the potential number of ORFs the virus may encode by ˜3 fold (1). The inventors designed a comprehensive screening approach to assess potential T cell responses against 563 of these ORFs, which included both previously reported and potentially novel HCMV proteins. 2593 15-mer peptides were predicted using computational algorithms, and a high throughput screen was performed using an IFNγ fluorospot assay to identify epitopes targeted by both CD8+ and CD4+ T cells in healthy HCMV-infected adults. This ‘whole ORFeome’ approach resulted in the identification of >200 new CD4+ and CD8+ T cell epitopes.

### Example 2: Targets of HCMV T Cell Reactivity

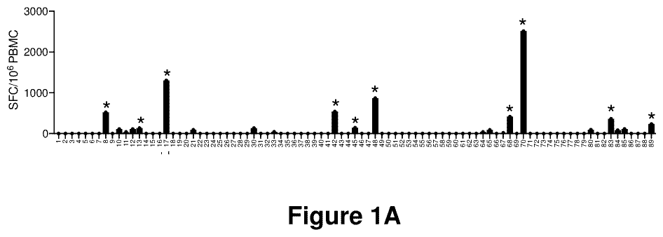

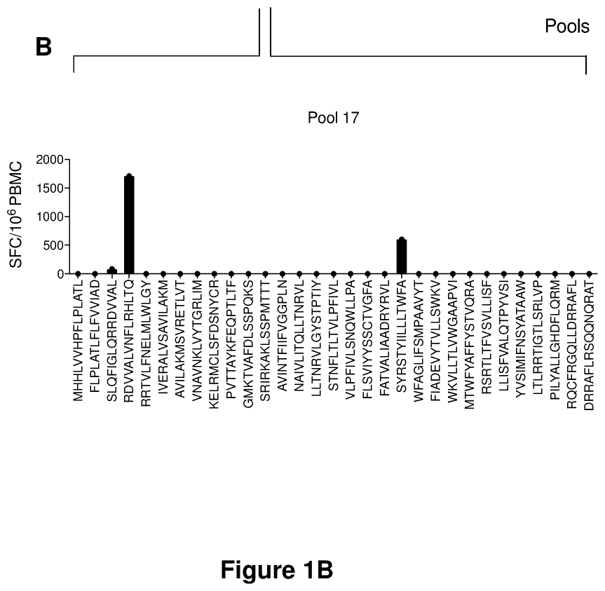

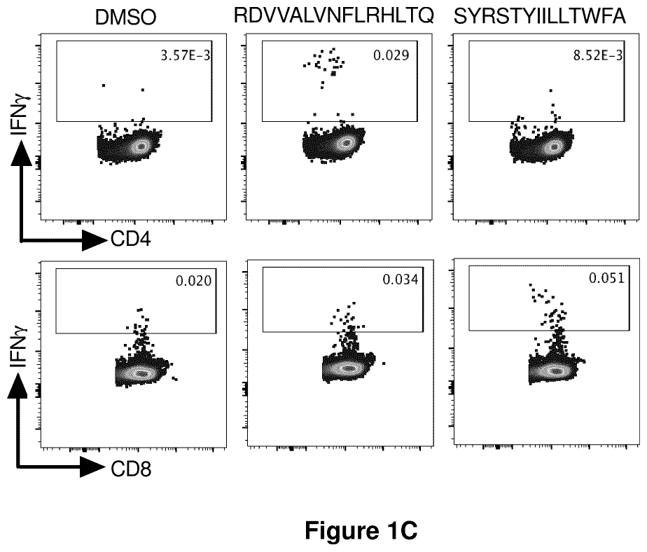

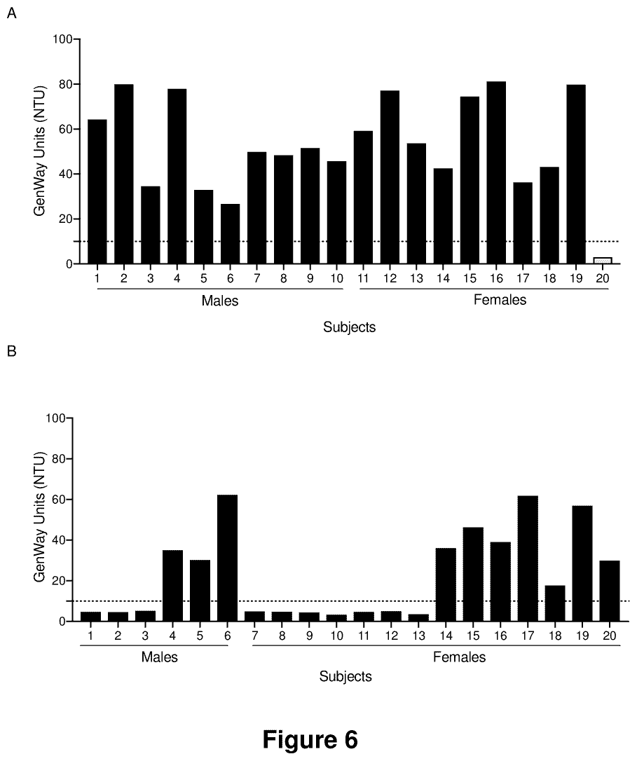

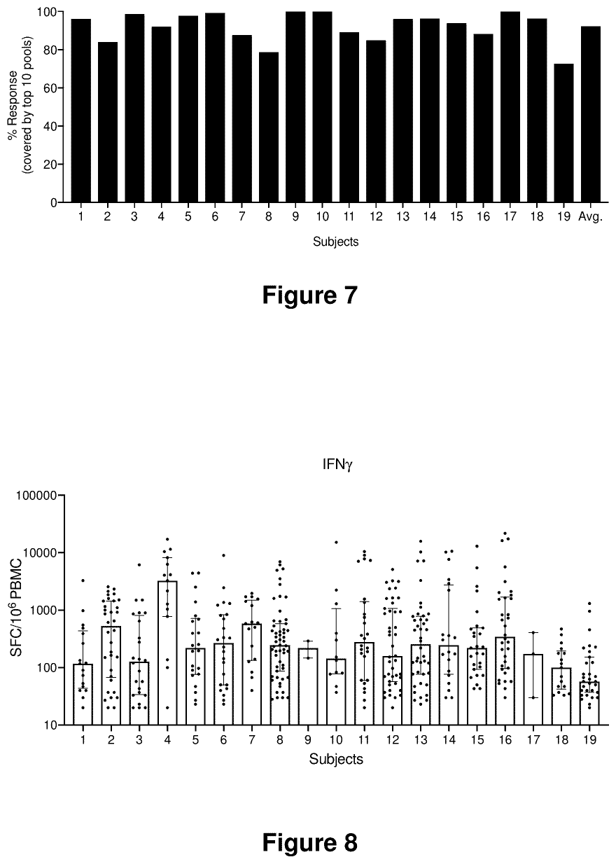

To define the targets of HCMV-specific T cell epitopes recognized in healthy adults, the inventors screened PBMCs of 19 subjects, 10 males and 9 females, recruited from San Diego blood bank (SDBB). The HCMV seropositivity of all the subjects was confirmed by IgG ELISA (FIG. 6A). A total of 2593 HCMV peptides covering a total of 563 ORFs (1) were tested. However, as many of these predicted ORFs overlapped 100% with others, as they were internal to longer ORFs, these 563 contained 359 completely unique ORFs composed of ˜150“canonical” ORFs, and an additional ˜200 identified by ribosomal RNA profiling (1). These peptides corresponded to predicted dominant epitopes, based on a bioinformatic method that predicts promiscuous binding to HLA class II molecules (40). Each of the ORFs above was covered by multiple predicted epitopes (with a minimum of at least 2), with the exception of very small ORFs (less than 15-20 amino acid residues), in which case at least one peptide was synthetized. The 2593 peptides were arranged in 89 pools of 28-30 15-mer peptides. PBMC reactivity of each of the 89 pools was assayed directly ex vivo using an IFN-γ Fluorospot assay. After identification of pools that resulted in IFN-γ production in HCMV+ individuals, the top 10 positive pools in each subject, which accounted for >90% of the total response (FIG. 7) were deconvoluted to identify specific epitopes. Representative results from the initial screening and the deconvolution of a pool in a representative subject are shown in FIG. 1A-B. In conclusion, the results shown here indicate that human T cell responses to HCMV recognize a wide breadth of different epitope specificities.

### Example 3: Characterization of CMV Epitope-Specific Immune Responses

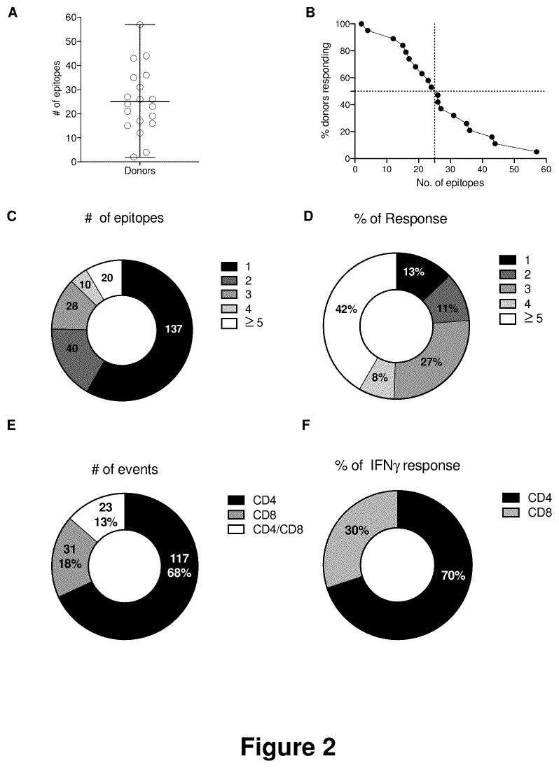

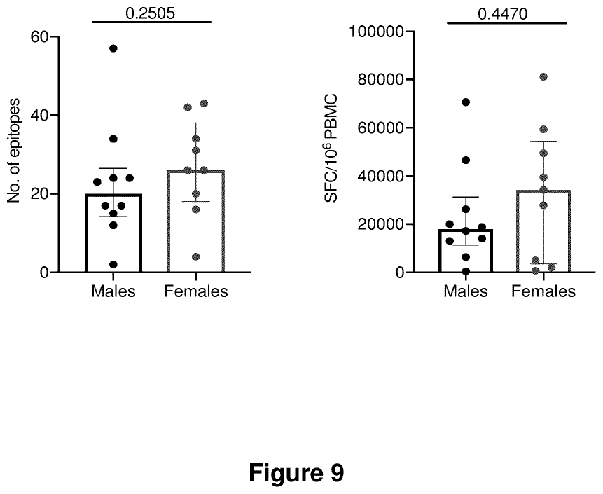

The deconvolution of the top 10 pools from each subject identified widespread reactivity directed against 235 unique epitopes (FIG. 8 and Table 1). Interestingly, females tended to show higher numbers and magnitude of epitope-specific responses compared to males, although this did not reach statistical significance (FIG. 9). On average each subject recognized 25 epitopes (FIG. 2A), and all subjects recognized at least 2 (range 2-57, FIG. 2B). Specifically, 6 out of 19 donors recognized 21-30 epitopes. A quarter of the epitopes (58 of the 235 recognized) were recognized by three or more subjects (FIG. 2C), and these accounted for 77% of the total response (FIG. 2D).

The inventors further characterized the phenotype of T cell responses directed against these 58 dominant epitopes by intracellular IFN-γ staining (representative results shown in FIG. 1C). In the majority of tested subjects, the responding T cells were CD4+; more specifically, in 68% the responding T cells were only CD4+ T cells, and in 13% the responding cells were both CD4+ and CD8+. In 18% of the cases the responses were mediated only by CD8+(FIG. 2E). Similarly, if the magnitude of the response was considered, 70% of the IFN-γ response was attributed to CD4+ T cells and only 30% emanated from CD8+ T cells (FIG. 2F). The fact that the responses were dominated by CD4+ T cells is consistent with the fact that the peptides tested were originally selected based on their predicted likelihood to bind HLA class II alleles. In turn, the occasional identification of epitope-specific CD8+ T cell responses in many cases likely reflects class I epitopes nested within the 15-mer epitopes tested in the screen. Overall, these results indicate that as expected, the screening strategy employed mostly identifies targets of CD4+ T cell reactivity.

### Example 4: Analysis of the ORF of Origin of the Identified Epitopes

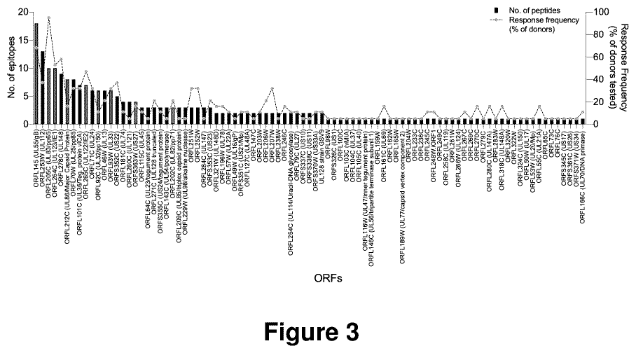

The 235 epitopes identified mapped to a total of 89 of the 359 unique ORFs screened. Of those, 28 ORFs contained >3 immunogenic peptides and 19 ORFs were recognized in 15% or more of the donors (FIG. 3). Notably, the previously well-characterized immunodominant ORFs such as envelope glycoprotein B (UL55), tegument proteins pp65/UL83, IE1 (UL123), major capsid protein UL86, IE2 (UL122), and pp150 (UL32) were amongst those associated with more than three immunogenic peptides.

To address the novelty of these findings, the results were compared with ORFs that have already been reported and curated in the Immune epitope database (IEDB htp://www.iedb.org) (41), as a source of defined epitopes. Specifically, a query of the IEDB revealed 7 ORFs that were previously extensively characterized as targets of T cell responses, and also tested in at least 19 donors and with a minimum of 15% frequency of positive responses, the conditions of our screening results: UL83/pp65 (ORFL205C), UL123/IE1 (ORFL264C), UL122/IE2 (ORFL265C), UL55/gB (ORFL145C), UL32/pp150 (ORFL92C), UL40 (ORFL105C) and UL98 (ORFL229W).

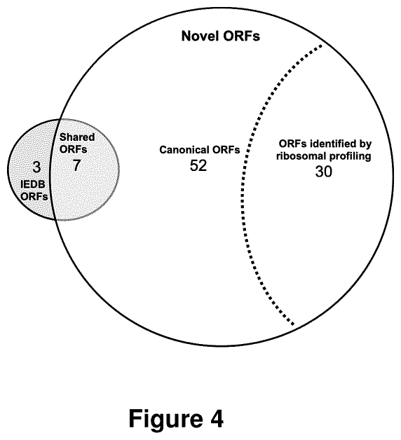

The same query revealed three ORFS that were not identified in the present screen. These ORFs were associated with a limited number of literature-reported and IEDB curated epitopes: UL75/gH (ORFL184C; 1 epitope), UL44 (ORFL112C.iORF1; 3 epitopes) and UL138 (ORFL313C; 1 epitope). Importantly, the present screen identified 82 ORFs that were not previously described as targets of T cell responses (FIG. 4).

Notably, 52 of these 82 ORFs were already described in the ‘canonical HCMV’ annotated genome, but all have not been described as targets of human T cell responses. Even more strikingly, 30 of these 82 ORFs corresponded to those mRNAs only identified by recent ribosomal profiling studies (1), formally proving that these mRNAs are translated in HCMV infected cells. These results indicate that the present approach successfully re-identified known ORFs as targets of T cell responses, and most importantly, greatly expanded the repertoire of canonical and novel ORFs recognized by T cell responses in healthy adults.

### Example 5: Novel Identified Epitope Pools Elicit Antigen Specific CD4+ T Cell Responses

The inventors explored whether the epitopes identified in the presented study could, alone or in combination with previously described epitopes, be utilized to generate epitope “MegaPools'” (MP) (42-46) which would allow to detect CMV-specific CD4 T cell responses. Accordingly, we generated a ‘P235’ MP encompassing the CMV 235 epitopes identified in the present study. As comparators, the commercially available CMV peptide pool (Mabtech, catalog 3619-1) encompassing a total of 42 CD4 and CD8 epitopes was considered. Additionally, we synthetized an MP including known class II epitopes curated in the IEDB database, encompassing a total of 187 CD4 epitopes (IEDB-II, Table 2).

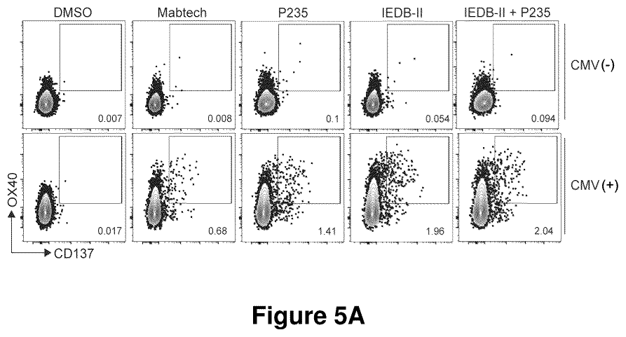

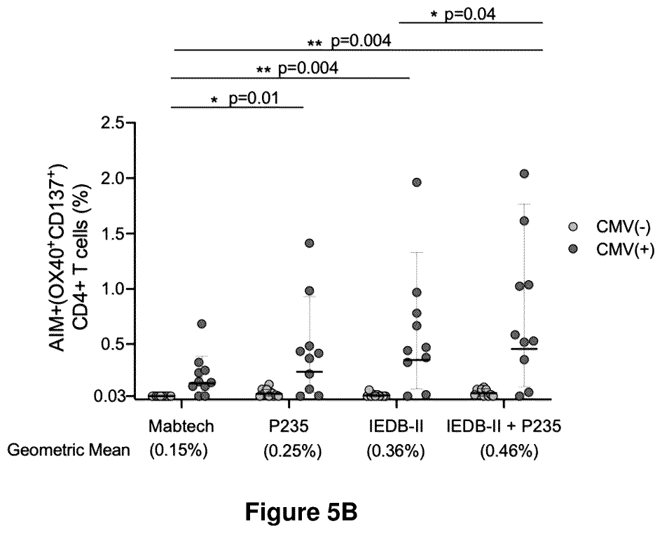

These MPs were tested with PBMC from a new cohort of 20 individuals (6 males and 14 females), which included both HCMV seropositive and seronegative donors (10 CMV+ and 10 CMV⁻, FIG. 6B for IgG ELISA CMV confirmation). PBMC from none of these subjects were used in the original epitope mapping experiments. PBMCs were stimulated with the Mabtech, P235, IEDB-II, or a combination of both P235/IEDB-II MPs. CD4+ T cell responses were measured as percentage of activation-induced marker assay positive (OX40+CD137+) CD4+ T cells and results are displayed in FIG. 5.

All CMV MPs tested were associated with significantly higher CD4 AIM responses in CMV+ individuals compared to CMV− subjects as shown in FIG. 5. Furthermore, and as expected due to the Mabtech pool containing fewer epitopes which are mainly CD8 T cell specific, when comparing seropositive AIM responses between the CMV pools, the P235, IEDB-II and P235/IEDB-II MPs were associated with significantly higher CMV specific CD4 responses (geometric mean 0.15% vs 0.25% CD4 AIM+, p=0.01; and 0.15% vs 0.36%, p=0.004, and 0.15% vs 0.46% CD4 AIM+, p=0.004, respectively). Additionally, the combination of the P235 and IEDB-II MPs elicited higher CD4 responses than either MP alone (0.39% vs 0.56% median CD4 AIM+, p=0.04 and 0.46% vs 0.56% median CD4 AIM+, p=0.0078, respectively) and had the highest magnitude response of all pools tested. This indicates that the combination of known (IEDB-II MP) and novel epitopes and ORFs (P235 MP) can capture the broadest range of CD4 T-cell responses and increased overall signal.

### Example 6

Embodiments of the present invention provide greater 200 new epitopes derived from >100 HCMV ORFs that induce virus-specific T cell responses. Importantly, this demonstrates that the current HLA peptide-binding prediction algorithms that have been refined over the last several decades are extremely efficient (49-53), and represent an excellent alternative to synthesizing genome-wide overlapping peptides, especially for large pathogens such as CMV. Despite the significant diversity in the human HLA repertoire, current advances in algorithm-based epitope identification take into consideration epitopes with the potential binding to diverse haplotypes, which undoubtedly contributed to this success (40, 54). Together, this approach allowed the inventors to increase the known T cell epitope landscape for HCMV by greater than 10-fold by synthesizing only 2593 peptides, illustrating both its efficiency and cost effectiveness for deciphering immune targets of large pathogens.

The inventors chose to use IFN-gamma production as a readout for positive epitope reactivity in a fluorospot-based assay to identify HCMV-specific T cell epitopes in this study. Like is true for most viral infections, CMV drives a strong Th1-like CD4+ response, and most effector and memory viral CD8+ T cells also produce this cytokine (55). However, future studies assessing which of these 235 epitopes may elicit HCMV-specific CD4 T cells to produce other cytokines are merited. Previously we have observed that Dengue virus epitope-specific CD4+ T cells can produce both IFN-gamma and IL-10 (56), something we have also seen during acute CMV infection in mice (57), where IL-10 producing CD4+ T cells enhance the duration of viral persistence (58). Recent studies by the Wills and Moss groups show that subsets of HCMV epitope-specific CD4+ T cells can produce IL-10 and also display cytolytic markers (59, 60). The potential CTL activity of HCMV-specific CD4+ T cells has been postulated for many years (61), and our recent results showing that CMV epitope-specific CD4 T cells can directly kill in vivo support this hypothesis (62). Taken together, this identification of >200 new T cell epitopes that elicit IFNγ production in this study provide valuable new tools to dissect the phenotypes and effector functions of HCMV-specific CD4 T cells in cases of both healthy and immune compromised patients, and will also help instruct ongoing vaccine efforts.

Of the 89 ORFs which we show here to be sources of specific T cell epitopes, 30 were uniquely identified as ribosome-bound RNAs in HCMV infected fibroblasts (1), with these 30 yielding 33 unique epitopes. Notably, of these 30 ORFs, 17 are predicted to produce proteins <50 amino acids in length, and 7 contain non-ATG start codons. This is consistent with recent studies suggesting that short/‘cryptic’ mRNAs present in both virally infected and tumor cells can be translated, proteolytically processed and loaded onto HLA molecules, resulting in the induction of epitope-specific T cell responses (63-65). Interestingly, one of the larger 30 ORFs containing two newly identified T cell epitopes (ORFL147C, 476 amino acids) has very recently been shown to regulate RNA binding/processing, and its deletion compromises CMV replication in fibroblasts (66). Despite >10% of the novel T cell epitopes identified here being derived from these newly described, ribosome-associated HCMV RNAs, no more than 2 of the 19 healthy donors analyzed produce T cells specific for any single one of these epitopes. This indicates that these novel ORFs 1) may not be broad targets of T cell responses in infected persons, 2) that specific individuals may more efficiently present epitopes derived from short/cryptic HCMV RNAs or 3) that minor HLA molecules may present them, with other possibilities also existing. Additionally, whether the proteins derived from these short ORFs are stable and play a role in the HCMV lifecycle remains an open question. Finally, we also identified 24 epitopes derived from 14 ‘canonical’ HCMV ORFs where the only historic support for their existence was the presence of their RNA from infected cells or bioinformatic analyses. Notably, a recent comprehensive study where 169 predicted canonical HCMV proteins (including these 14) were epitope-tagged, expressed stably in infected cells, immunoprecipitated and analyzed for interacting proteins by mass spectrometry supports our results that these ORFs are expressed as proteins (66).

Of the 52 canonical ORFs that we have identified here to contain T cell epitopes, >25% of these are known to function as immunomodulatory proteins (67). This is intriguing, as perhaps these HCMV proteins are more subject to being localized to antigen-processing or presentation compartments within infected cells. One of these epitopes is derived from the HCMV IL-10 orthologue, which is being considered as a potential HCMV vaccine candidate (68, 69). Additionally, 3 epitopes were found to be imbedded within the viral UL128 protein, a critical component of the pentameric envelope protein complex (UL128-131/gH/gL) that mediates entry of HCMV into non-fibroblast cell types (70, 71). This is also of high potential interest in the context of vaccine development, as many believe the pentamer should be included in a viral- or subunit-based approach (72). Notably, both vIL-10 and UL128 have largely been considered only in the context of their abilities to induce antibody-based vaccine protection, but our identification of T cell epitopes derived from both these HCMV proteins suggests they may function to prime both humoral and cellular immunity.

### Methods

### Study Design

For the initial CMV ORF screen, the responses of 19 CMV-seropositive subjects were evaluated. PBMCs were stimulated with 89 pools covering 563 ORFs of HCMV. Each pool comprised of 28-30 15-mer peptides overlapping by 10 residues. PBMCs that were found reactive to a pool were further tested against individual peptides contained in the pool using IFN-γ Fluorospot assay. To further characterize epitopes presented to CD8+ and CD4+ T cells, flow cytometry was used to detect IFN-γ production by PBMCs that were stimulated with individual peptides against which response was observed in IFN-γ fluorospot assay.

For the CMV-235 validation and comparison screen, the responses of anew cohort consisting of 23 CMV-seropositive and 16 seronegative subjects were evaluated. PBMCs were stimulated with CMV-Mabtech peptide pool (Catalog 3619-1), CMV-IEDB peptide pool (Table x) (44, 46), CMV-235 pool, or a combination of both CMV-IEDB and CMV-235 pools. PBMC responses were assayed using the same IFN-γ Fluorospot assay. These studies were approved by the institutional review board committee at La Jolla Institute protocol number: VD-112 and VD-174.

### Subjects

19 subjects (10 males and 9 females) were recruited anonymously from San Diego blood bank (SDBB) for the initial CMV ORF screens. For the CMV235 comparison screens, samples from 39 subjects (13 males and 26 females) were obtained by La Jolla Institute Clinical Core and Continental Services Group (Miami, FL) for prior, unrelated studies. Blood samples were collected by trained staff. At the time of enrollment in the initial studies, all individual subjects provided informed consent that any leftover sample could be used for future studies, which includes this study. These subjects were considered healthy as defined by no known history of any significant systemic diseases (not limited to autoimmune disease, diabetes, kidney or liver disease, congestive heart failure, malignancy, coagulopathy, hepatitis B or C, or HIV). The demographics of those subjects are provided in Table 3.

The IgG antibodies of the subjects for both cohorts was measured using Cytomegalovirus IgG Elisa kit from Genway Biotech Inc. according to manufacturer's instructions.

TABLE 3
Demographic characteristics of CMV (+/−) subjects
analyzed in screening and validation studies.

| | Screening cohort | | Validation cohort | | | |
|---|---|---|---|---|---|---|
| Characteristics | CMV positive | | CMV positive | | CMV negative | |
| Total participants enrolled, n | 19 | | 10 | | 10 | |
| Males/females | 10/9 | | 3/7 | | 3/7 | |
| Median age (range) | 65 | (28-80) | 35.5 | (22-55) | 28.5 | (19-42) |
| Caucasian, % (n) | 68 | (13) | 40 | (4) | 40 | (4) |
| Asian, % (n) | 16 | (3) | 10 | (1) | 20 | (2) |
| African American, % (n) | 5 | (1) | 10 | (1) | 10 | (1) |
| More than one race, % (n) | 0 | (0) | 30 | (3) | 30 | (3) |
| Unknown, % (n) | 10 | (2) | 10 | (1) | 0 | (0) |

Peptide prediction

Based on the 7-allele method as previously described (40), 2593 peptides were predicted for 563 potential HCMV ORFs. Of the 751 ORFs predicted by ribosomal profiling (1), those smaller than 15 amino acids were excluded, and only one peptide of 15-20 amino acids in length was selected for screening.

### Peptide Libraries and Pool Preparation

The predicted peptides were commercially synthesized as crude material by A&A ltd, San Diego. The peptides were solubilized in DMSO at a concentration of 20 mg/ml and spot checked for quality by mass spectrometry. The peptides were pooled into peptide pools containing 28-30 peptides constituting multiple ORFs per pool. A total of 89 pools were prepared covering 563 ORFs of HCMV. The final concentration of each pool was 0.7 mg/ml.

For the IEDB-II (Table x) and CMV235 (Table x) peptide pools peptides were synthesized by A&A ltd, San Diego, resuspended in DMSO, pooled and sequentially lyophilized as previously described (47). The IEDB-II peptide pool was developed based on data available in the IEDB (www.iedb.org) (41). The MHC class II restricted epitopes for CMV was extracted from the IEDB using the following query; Organism: human herpesvirus 5 (ID:10359), positive assays only, no B cell assays, MHC restriction type: class II, host: Homo sapiens. The resulting 187 epitopes (table x) were filtered for size (13-20 amino acids) and discovered using one of the following assays: ELISPOT, ICS, multi- or tetramers, proliferation and “helper response”. The CMV peptide pool for human CD4 and CD8 T cells containing 42 peptides (14 MHC class II restricted and 28 MHC class I restricted) representing pp50, pp65, IE1, IE2, and envelope glycoprotein B was purchased from Mabtech.

### Isolation of PBMC by Ficoll-Paque Density Gradient Centrifugation

1 unit blood from each donor was processed for PBMC isolation. Briefly, blood was centrifuged at for and the top layer of plasma was removed. The remaining blood was diluted and layered over 15 ml of Ficoll-Paque. Tubes were spun at room temperature in a swinging bucket rotor without brake applied. The PBMC interface was carefully removed by pipetting and washed with PBS by centrifugation at 800 rpm for 10 mins with brakes off. PBMC pellet was resuspended in RPMI media, cell number and viability were determined by trypan blue staining and cells were cryopreserved in liquid nitrogen in freezing media (90% Fetal bovine serum and 10% DMSO) at a density of 30 million/ml and stored until further processed.

### Fluorospot Assay

PBMC were thawed, washed and counted for viability using the trypan blue exclusion method. 200,000 cells were plated in triplicates and stimulated with pools (2 ug/ml) or peptides (10 ug/ml), PHA (10 ug/ml) or medium containing equivalent amount of DMSO in 96-well plates (Immubilion-P, Millipore) previously coated with anti IFN-γ antibody (1-DIK, Mabtech, Stockholm, Sweden). After 20 hr incubation at 37° C., cells were discarded and wells were washed six times with PBS/0.05% Tween 20 using an automated plate washer and further incubated with IFN-γ antibody (7-B6-1-FS-BAM) for 2 hrs at room temperature. After incubation, wells were washed and incubated with fluorophore conjugated anti-BAM-490 antibody for 1 hr at room temperature. Finally, the plates were washed and incubated with fluorescence enhancer for 15 min, blotted dry and fluorescent spots were counted by computer assisted image analysis (IRIS Fluorospot reader, Mabtech, Sweden).

Each pool or peptide was considered positive compared to the background that had equivalent amount of DMSO based on the following criteria: (i) 20 or more spot forming cells (SFC) per 10⁶ PBMC after background subtraction, (ii) the stimulation index greater than 2, and (iii) p<0.05 by student's t test or Poisson distribution test

### Intracellular Cytokine Assay for IFN-γ

Intracellular staining for IFN-gamma and flow cytometry was performed to detect antigen specific T cell responses. 1×10⁶ PBMCs suspended in RPMI medium supplemented with 1-% heat inactivated human AB serum, glutamine and penicillin streptomycin were plated in U-bottom 96 well plates. After overnight resting at 37° C., PBMCs were spun and replaced with fresh RPMI media and stimulated with individual peptides at a concentration of 10 μg/ml. PHA at a concentration of 5 μg/ml was used as a positive control. After 1 hr of incubation at 37° C., 2 g/ml of Brefeldin was added and cell were further incubated at 37° C. for additional 5 hrs. The cells were then harvested, washed with 200 μl of MACS Buffer and stained with a cocktail of antibodies that contained CD3-Af700, CD4-APCef780, CD8-BV650, CD14-V500, CD19-V500, and fixable viability dye-e506 for 30 min at 4° C. The cells were then washed thrice with 200 μl MACS buffer, fixed using 4% PFA for 10 mins at 4° C., washed with 200 μl PBS and rested at 4° C. overnight in 200 μl MACS buffer. The following day, cells were washed, permeabilized by washing with 200 μl saponin buffer (0.5% saponin in PBS), washed with blocking buffer (10% human serum prepared in saponin buffer) and stained with IFN-γ-FITC antibody at room temperature for 30 mins. The cells were finally washed with PBS and suspended in 200 μl PBS.

The cells were acquired on ZE5 Biorad plate reader and further analysis was done on Flowjo software. Gates were applied on live single cells for CD3+, CD4+ and CD8+ T cell populations. The percentage of reactive CD4+ or CD8+ IFN-γ T cells were expressed as a percent of the total number of parent population analyzed. Reactive populations met the following 2 criteria: (i) well-defined cell population positive for both IGN-y and CD4 or CD8 constituting at least 0.02% of the total number of CD4+ or CD8+ cells analyzed (ii) stimulation index greater than 2.

### Activation Induced Marker (AIM) Assay

PBMC were thawed, washed and counted for viability using the trypan blue exclusion method. 1 million cells per donor/condition were plated and cultured in the presence of the CMV specific pools (1 ug/mL for CMV-235 and IEDB-II, 2 ug/mL for Mabtech pool), PHA (10 ug/mL), or medium containing equivalent amount of DMSO in 96-well U bottom plates. Cells were then harvested, washed with 200 ul of MACS Buffer and stained with a cocktail of antibodies that contained CD3-Af700, CD4-BV605, CD8-PerCP-Cy5.5, CD14-V500, CD19-V500, OX40-PE-Cy7, CD137-APC, and fixable viability dye-e506 for 30 min at 4° C. The cells were then washed thrice with 200 μl MACS buffer, fixed using 4% PFA for 10 mins at 4° C., and resuspended in 200 μl of PBS for acquisition.

Cells were acquired on a BD LSRFortessa and further analysis was done on Flowjo software. As previously described (44, 48), quantification of live, singlet antigen specific CD4 T cells was determined as a percentage of their OX40+CD137+ expression (AIM+). CMV specific AIM+CD4 T cell signals were background subtracted with their corresponding negative control DMSO samples, with a minimal DMSO level set to 0.005%. The limit of detection (LOD) for the AIM+ assay was calculated by multiplying the upper confidence interval of the geometric mean of all DMSO samples by 2 (0.03).

### Statistical Analysis

Statistical analyses were performed using GraphPad Prism version 8.1.1

### Definitions

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., sgRNA) may be detected by standard PCR or Northern blot methods well known in the art. See, Sambrook et al., 1989 Molecular Cloning: A Laboratory Manual, 18.1-18.88.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, y-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “polypeptide,” “peptide” and “protein” are used interchangeably herein to refer to a polymer of amino acid residues, wherein the polymer may, in embodiments, be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. A “fusion protein” refers to a chimeric protein encoding two or more separate protein sequences that are recombinantly expressed as a single moiety.

Proteins and peptides include isolated and purified forms. Proteins and peptides also include those immobilized on a substrate, as well as amino acid sequences, subsequences, portions, homologues, variants, and derivatives immobilized on a substrate.

Proteins and peptides can be included in compositions, for example, a pharmaceutical composition. In particular embodiments, a pharmaceutical composition is suitable for specific or non-specific immunotherapy, or is a vaccine composition.

Isolated nucleic acid (including isolated nucleic acid) encoding the proteins and peptides are also provided. Cells expressing a protein or peptide are further provided. Such cells include eukaryotic and prokaryotic cells, such as mammalian, insect, fungal and bacterial cells.

Methods and uses and medicaments of proteins and peptides of the invention are included. Such methods, uses and medicaments include modulating immune activity of a cell against a pathogen, for example, a bacteria or virus.

The term “peptide mimetic” or “peptidomimetic” refers to protein-like chain designed to mimic a peptide or protein. Peptide mimetics may be generated by modifying an existing peptide or by designing a compound that mimic peptides, including peptoids and β-peptides.

“Conservatively modified variants” applies to both amino acid and nucleic acid sequences. With respect to particular nucleic acid sequences, “conservatively modified variants” refers to those nucleic acids that encode identical or essentially identical amino acid sequences. Because of the degeneracy of the genetic code, a number of nucleic acid sequences will encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure. The following eight groups each contain amino acids that are conservative substitutions for one another: (1) Alanine (A), Glycine (G); (2) Aspartic acid (D), Glutamic acid (E); (3) Asparagine (N), Glutamine (Q); (4) Arginine (R), Lysine (K); (5) Isoleucine (I), Leucine (L), Methionine (M), Valine (V); (6) Phenylalanine (F), Tyrosine (Y), Tryptophan (W); (7) Serine (S), Threonine (T); and (8) Cysteine (C), Methionine (M) (see, e.g., Creighton, Proteins (1984)).

A “percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site ncbi.nlm.nih.gov/BLAST/or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

An amino acid or nucleotide base “position” is denoted by a number that sequentially identifies each amino acid (or nucleotide base) in the reference sequence based on its position relative to the N-terminus (or 5′-end). Due to deletions, insertions, truncations, fusions, and the like that must be taken into account when determining an optimal alignment, in general the amino acid residue number in a test sequence determined by simply counting from the N-terminus will not necessarily be the same as the number of its corresponding position in the reference sequence. For example, in a case where a variant has a deletion relative to an aligned reference sequence, there will be no amino acid in the variant that corresponds to a position in the reference sequence at the site of deletion. Where there is an insertion in an aligned reference sequence, that insertion will not correspond to a numbered amino acid position in the reference sequence. In the case of truncations or fusions there can be stretches of amino acids in either the reference or aligned sequence that do not correspond to any amino acid in the corresponding sequence.

The terms “numbered with reference to” or “corresponding to,” when used in the context of the numbering of a given amino acid or polynucleotide sequence, refers to the numbering of the residues of a specified reference sequence when the given amino acid or polynucleotide sequence is compared to the reference sequence.

The term “multimer” refers to a complex comprising multiple monomers (e.g., a protein complex) associated by noncovalent bonds. The monomers be substantially identical monomers, or the monomers may be different. In embodiments, the multimer is a dimer, a trimer, a tetramer, or a pentamer.

As used herein, the term “Major Histocompatibility Complex” (MHC) is a generic designation meant to encompass the histocompatibility antigen systems described in different species including the human leucocyte antigens (HLA). Typically, MHC Class I or Class II multimers are well known in the art and include but are not limited to dimers, tetramers, pentamers, hexamers, heptamers and octamers.

As used herein, the term “MHC/peptide multimer” refers to a stable multimeric complex composed of MHC protein(s) subunits loaded with a peptide of the present invention. For example, an MHC/peptide multimer (also called herein MHC/peptide complex) include, but are not limited to, an MHC/peptide dimer, trimer, tetramer, pentamer or higher valency multimer. In humans there are three major different genetic loci that encode MHC class I molecules (the MHC molecules of the human are also designated human leukocyte antigens (HLA)): HLA-A, HLA-B, HLA-C, e.g., HLA-A*01, HLA-A*02, and HLA-A*11 are examples of different MHC class I alleles that can be expressed from these loci. Non-classical human MHC class I molecules such as HLA-E (homolog of mice Qa-1b) and MICA/B molecules are also encompassed by the present invention. In some embodiments, the MHC/peptide multimer is an HLA/peptide multimer selected from the group consisting of HLA-A/peptide multimer, HLA-B/peptide multimer, HLA-C/peptide multimer, HLA-E/peptide multimer, MICA/peptide multimer and MICB/peptide multimer.

In humans there are three major different genetic loci that encode MHC class II molecules: HLA-DR, HLA-DP, and HLA-DQ, each formed of two polypeptides, alpha and beta chains (A and B genes). For example, HLA-DQA1*01, HLA-DRB1*01, and HLA-DRB1*03 are different MHC class II alleles that can be expressed from these loci. It should be further noted that non-classical human MHC class II molecules such as HLA-DM and HL-DOA (homolog in mice is H2-DM and H2-0) are also encompassed by the present invention. In some embodiments, the MHC/peptide multimer is an HLA/peptide multimer selected from the group consisting of HLA-DP/peptide multimer, HLA-DQ/peptide multimer, HLA-DR/peptide multimer, HLA-DM/peptide multimer and HLA-DO/peptide multimer.

An MHC/peptide multimer may be a multimer where the heavy chain of the MHC is biotinylated, which allows combination as a tetramer with streptavidin. MHC-peptide tetramers have increased avidity for the appropriate T cell receptor (TCR) on T lymphocytes. The multimers can also be attached to paramagnetic particles or magnetic beads to facilitate removal of non-specifically bound reporter and cell sorting. Multimer staining does not kill the labelled cells, thus, cell integrity is maintained for further analysis. In some embodiments, the MHC/peptide multimer of the present invention is particularly suitable for isolating and/or identifying a population of CD8+ T cells having specificity for the peptide of the present invention (in a flow cytometry assay).

The peptides or MHC class I or class II multimer as described herein is particularly suitable for detecting T cells specific for one or more peptides of the present invention. The peptide(s) and/or the MHC/multimer complex of the present invention is particularly suitable for diagnosing cytomegalovirus infection in a subject. For example, the method comprises obtaining a blood or PBMC sample obtained from the subject with an amount of a least peptide of the present invention and detecting at least one T cell displaying a specificity for the peptide. Another diagnostic method of the present invention involves the use of a peptide of the present invention that is loaded on multimers as described above, so that the isolated CD8+ or CD4+ T cells from the subject are brought into contact with the multimers, at which the binding, activation and/or expansion of the T cells is measured. For example, following the binding to antigen presenting cells, e.g., those having the MHC class I or class II multimer, the number of CD8+ and/or CD4+ cells binding specifically to the HLA-peptide multimer may be quantified by measuring the secretion of lymphokines/cytokines, division of the T cells, or standard flow cytometry methods, such as, for example, using fluorescence activated cell sorting (FACS). The multimers can also be attached to paramagnetic ferrous or magnetic beads to facilitate removal of non-specifically bound reporter and cell sorting.

The MHC class I or class II peptide multimers as described herein can also be used as therapeutic agents. The peptide and/or the MHC class I or class II peptide multimers of the present invention are suitable for treating or preventing a cytomegalovirus infection in a subject. The MHC Class I or Class II multimers can be administered in soluble form or loaded on nanoparticles.

The term “antibody” refers to a polypeptide encoded by an immunoglobulin gene or functional fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

The phrase “specifically (or selectively) binds” to an antibody or “specifically (or selectively) immunoreactive with,” when referring to a protein or peptide, refers to a binding reaction that is determinative of the presence of the protein or peptide, often in a heterogeneous population of proteins and other biologics. Thus, under designated immunoassay conditions, the specified antibodies bind to a particular protein at least two times the background and more typically more than 10 to 100 times background. Specific binding to an antibody under such conditions requires an antibody that is selected for its specificity for a particular protein. For example, polyclonal antibodies can be selected to obtain only a subset of antibodies that are specifically immunoreactive with the selected antigen and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998) for a description of immunoassay formats and conditions that can be used to determine specific immunoreactivity).

Antibodies are large, complex molecules (molecular weight of ˜150,000 or about 1320 amino acids) with intricate internal structure. A natural antibody molecule contains two identical pairs of polypeptide chains, each pair having one light chain and one heavy chain. Each light chain and heavy chain in turn consists of two regions: a variable (“V”) region involved in binding the target antigen, and a constant (“C”) region that interacts with other components of the immune system. The light and heavy chain variable regions come together in 3-dimensional space to form a variable region that binds the antigen (for example, a receptor on the surface of a cell). Within each light or heavy chain variable region, there are three short segments (averaging 10 amino acids in length) called the complementarity determining regions (“CDRs”). The six CDRs in an antibody variable domain (three from the light chain and three from the heavy chain) fold up together in 3-dimensional space to form the actual antibody binding site which docks onto the target antigen. The position and length of the CDRs have been precisely defined by Kabat, E. et al., Sequences of Proteins of Immunological Interest, U.S. Department of Health and Human Services, 1983, 1987. The part of a variable region not contained in the CDRs is called the framework (“FR”), which forms the environment for the CDRs.

The term “antibody” is used according to its commonly known meaning in the art. Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into a Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (VL) and variable heavy chain (VH) refer to these light and heavy chains respectively. The Fc (i.e., fragment crystallizable region) is the “base” or “tail” of an immunoglobulin and is typically composed of two heavy chains that contribute two or three constant domains depending on the class of the antibody. By binding to specific proteins, the Fc region ensures that each antibody generates an appropriate immune response for a given antigen. The Fc region also binds to various cell receptors, such as Fc receptors, and other immune molecules, such as complement proteins.

As used herein, the term “antigen” and the term “epitope” refers to a molecule or substance capable of stimulating an immune response. In one example, epitopes include but are not limited to a polypeptide and a nucleic acid encoding a polypeptide, wherein expression of the nucleic acid into a polypeptide is capable of stimulating an immune response when the polypeptide is processed and presented on a Major Histocompatibility Complex (MHC) molecule. Generally, epitopes include peptides presented on the surface of cells non-covalently bound to the binding groove of Class I or Class II MHC, such that they can interact with T cell receptors and the respective T cell accessory molecules. However, antigens and epitopes also apply when discussing the antigen binding portion of an antibody, wherein the antibody binds to a specific structure of the antigen.

Proteolytic Processing of Antigens. Epitopes that are displayed by MHC on antigen presenting cells are cleavage peptides or products of larger peptide or protein antigen precursors. For MHC I epitopes, protein antigens are often digested by proteasomes resident in the cell. Intracellular proteasomal digestion produces peptide fragments of about 3 to 23 amino acids in length that are then loaded onto the MHC protein. Additional proteolytic activities within the cell, or in the extracellular milieu, can trim and process these fragments further. Processing of MHC Class II epitopes generally occurs via intracellular proteases from the lysosomal/endosomal compartment. The present invention includes, in one embodiment, pre-processed peptides that are attached to the anti-CD40 antibody (or fragment thereof) that directs the peptides against which an enhanced immune response is sought directly to antigen presenting cells.

The present invention includes methods for specifically identifying the epitopes within antigens most likely to lead to the immune response sought for the specific sources of antigen presenting cells and responder T cells.

As used herein, the term “T cell epitope” refers to a specific amino acid that when present in the context of a Major or Minor Histocompatibility Complex provides a reactive site for a T cell receptor. The T-cell epitopes or peptides that stimulate the cellular arm of a subject's immune system are short peptides of about 8-25 amino acids. T-cell epitopes are recognized by T cells from animals that are immune to the antigen of interest. These T-cell epitopes or peptides can be used in assays such as the stimulation of cytokine release or secretion or evaluated by constructing major histocompatibility (MHC) proteins containing or “presenting” the peptide. Such immunogenically active fragments are often identified based on their ability to stimulate lymphocyte proliferation in response to stimulation by various fragments from the antigen of interest.

As used herein, the term “immunological response” refers to an antigen or composition is the development in a subject of a humoral and/or a cellular immune response to an antigen present in the composition of interest. For purposes of the present disclosure, a “humoral immune response” refers to an immune response mediated by antibody molecules, while a “cellular immune response” is one mediated by T-lymphocytes and/or other white blood cells. One important aspect of cellular immunity involves an antigen-specific response by cytolytic T-cells (“CTL”s). CTLs have specificity for peptide antigens that are presented in association with proteins encoded by the major histocompatibility complex (MHC) and expressed on the surfaces of cells. CTLs help induce and promote the destruction of intracellular microbes, or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+ T-cells. Hence, an immunological response may include one or more of the following effects: the production of antibodies by B-cells; and/or the activation of effector and/or suppressor T-cells and/or gamma-delta T-cells directed specifically to an antigen or antigens present in the composition or vaccine of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art.

As used herein, the term an “immunogenic composition” and “vaccine” refer to a composition that comprises an antigenic molecule where administration of the composition to a subject or patient results in the development in the subject of a humoral and/or a cellular immune response to the antigenic molecule of interest. “Vaccine” refers to a composition that can provide active acquired immunity to and/or therapeutic effect (e.g., treatment) of a particular disease or a pathogen. A vaccine typically contains one or more agents that can induce an immune response in a subject against a pathogen or disease, i.e., a target pathogen or disease. The immunogenic agent stimulates the body's immune system to recognize the agent as a threat or indication of the presence of the target pathogen or disease, thereby inducing immunological memory so that the immune system can more easily recognize and destroy any of the pathogen on subsequent exposure. Vaccines can be prophylactic (e.g., preventing or ameliorating the effects of a future infection by any natural or pathogen) or therapeutic (e.g., reducing symptoms or aberrant conditions associated with infection). The administration of vaccines is referred to vaccination.

In some examples, a vaccine composition can provide nucleic acid, e.g., mRNA that encodes antigenic molecules (e.g., peptides) to a subject. The nucleic acid that is delivered via the vaccine composition in the subject can be expressed into antigenic molecules and allow the subject to acquire immunity against the antigenic molecules. In the context of the vaccination against infectious disease, the vaccine composition can provide mRNA encoding antigenic molecules that are associated with a certain pathogen, e.g., one or more peptides that are known to be expressed in the pathogen (e.g., pathogenic bacterium or virus).

The present invention provides nucleic acid molecules, specifically polynucleotides, primary constructs and/or mRNA that encode one or more polynucleotides that express one or more peptides or proteins, comprising, consisting of, or consisting essentially of an amino acid sequence selected from those sequences set forth in Table 1 or Table 2, or a subsequence, portion, homologue, variant or derivative thereof for use in immune modulation. The term “nucleic acid” refers to any compound and/or substance that comprise a polymer of nucleotides, referred to herein as polynucleotides. Exemplary nucleic acids or polynucleotides of the invention include, but are not limited to, ribonucleic acids (RNAs), deoxyribonucleic acids (DNAs), threose nucleic acids (TNAs), glycol nucleic acids (GNAs), peptide nucleic acids (PNAs), locked nucleic acids (LNAs), including diastereomers of LNAs, functionalized LNAs, or hybrids thereof.

One method of immune modulation of the present invention includes direct or indirect gene transfer, i.e., local application of a preparation containing the one or more polynucleotides (DNA, RNA, mRNA, etc.) that expresses the one or more peptides or proteins, comprising, consisting of, or consisting essentially of an amino acid sequence selected from those sequences set forth in Table 1 or Table 2, or a subsequence, portion, homologue, variant or derivative thereof. A variety of well-known vectors can be used to deliver to cells the one or more polynucleotides or the peptides or proteins expressed by the polynucleotides, including but not limited to adenoviral vectors and adeno-associated vectors. In addition, naked DNA, liposome delivery methods, or other novel vectors developed to deliver the polynucleotides to cells can also be beneficial. Any of a variety of promoters can be used to drive peptide or protein expression, including but not limited to endogenous promoters, constitutive promoters (e.g., cytomegalovirus, adenovirus, or SV40), inducible promoters (e.g., a cytokine promoter such as the interleukin-1, tumor necrosis factor-alpha, or interleukin-6 promoter), and tissue specific promoters to express the immunogenic peptides or proteins of the present invention.

The immunization may include adenovirus, adeno-associated virus, herpes virus, vaccinia virus, retroviruses, or other viral vectors with the appropriate tropism for cells likely to present the antigenic peptide(s) or protein(s) may be used as a gene transfer delivery system for a therapeutic peptide(s) or protein(s), comprising, consisting of, or consisting essentially of an amino acid sequence selected from those sequences set forth in Table 1 or Table 2, or a subsequence, portion, homologue, variant or derivative thereof, gene expression construct. Viral vectors which do not require that the target cell be actively dividing, such as adenoviral and adeno-associated vectors, are particularly useful when the cells are accumulating, but not proliferative. Numerous vectors useful for this purpose are generally known (Miller, Human Gene Therapy 15-14, 1990; Friedman, Science 244:1275-1281, 1989; Eglitis and Anderson, BioTechniques 6:608-614, 1988; Tolstoshev and Anderson, Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; and Miller and Rosman, Bio Techniques 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346).

The immunization may also include inserting the one or more polynucleotides (DNA, RNA, mRNA, etc.) that express the one or more peptides or proteins, comprising, consisting of, or consisting essentially of an amino acid sequence selected from those sequences set forth in Table 1 or Table 2, or a subsequence, portion, homologue, variant or derivative thereof into the viral vector, along with another gene which encodes the ligand for a receptor on a specific target cell, for example, such that the vector is now target specific. Viral vectors can be made target specific by attaching, for example, a sugar, a glycolipid, or a protein. Targeting can also be accomplished by using an antibody to target the viral vector. Those of skill in the art will know of, or can readily ascertain without undue experimentation, specific polynucleotide sequences which can be inserted into the viral genome or attached to a viral envelope to allow target specific delivery of the viral vector containing the gene.

Since recombinant viruses are defective, they require assistance in order to produce infectious vector particles. This assistance can be provided, for example, by using helper cell lines that contain plasmids encoding all of the structural genes of the virus under the control of regulatory sequences within the viral genome. These plasmids are missing a nucleotide sequence which enables the packaging mechanism to recognize a polynucleotide transcript for encapsidation. These cell lines produce empty virions, since no genome is packaged. If a viral vector is introduced into such cells in which the packaging signal is intact, but the structural genes are replaced by other genes of interest, the vector can be packaged and vector virion produced.

Viral or non-viral approaches may also be employed for the introduction of one or more therapeutic polynucleotides that express the one or more peptides or proteins, comprising, consisting of, or consisting essentially of an amino acid sequence selected from those sequences set forth in Table 1 or Table 2, or a subsequence, portion, homologue, variant or derivative thereof, into polynucleotide-encoding polynucleotide into antigen presenting cells. The polynucleotides may be DNA, RNA, mRNA that directly encode the one or more peptides or proteins of the present invention, or may be introduced as part of an expression vector.

Another example of an immunization includes colloidal dispersion systems that include macromolecule complexes, nanocapsules, microspheres, beads, and lipid-based systems including oil-in-water emulsions, micelles, mixed micelles, and liposomes and the one or more polynucleotides that express the one or more peptides or proteins, comprising, consisting of, or consisting essentially of an amino acid sequence selected from those sequences set forth in Table 1 or Table 2, or a subsequence, portion, homologue, variant or derivative thereof. One non-limiting example of a colloidal system for use with the present invention is a liposome. Liposomes are artificial membrane vesicles which are useful as delivery vehicles in vitro and in vivo. It has been shown that large unilamellar vesicles (LUV), which range in size from 0.2-4.0 micrometers that can encapsulate a substantial percentage of an aqueous buffer containing large macromolecules. RNA, DNA and intact virions can be encapsulated within the aqueous interior and be delivered to cells in a biologically active form (Fraley, et al., Trends Biochem. Sci., 6:77, 1981). In addition to mammalian cells, liposomes have been used for delivery of polynucleotides in plant, yeast and bacterial cells. In order for a liposome to be an efficient gene transfer vehicle, the following characteristics should be present: (Zakut and Givol, supra) encapsulation of the genes of interest at high efficiency while not compromising their biological activity; (Fearnhead, et al., supra) preferential and substantial binding to a target cell in comparison to non-target cells; (Korsmeyer, S. J., supra) delivery of the aqueous contents of the vesicle to the target cell cytoplasm at high efficiency; and (Kinoshita, et al., supra) accurate and effective expression of genetic information (Mannino, et al., Bio Techniques, 6:682, 1988).

The composition for immunizing the subject or patient may, in certain embodiments comprise a combination of phospholipid, particularly high-phase-transition-temperature phospholipids, usually in combination with steroids, especially cholesterol. Other phospholipids or other lipids may also be used. The physical characteristics of liposomes depend on pH, ionic strength, and the presence of divalent cations. The targeting of liposomes can be classified based on anatomical and mechanistic factors. Anatomical classification is based on the level of selectivity, for example, organ-specific, cell-specific, and organelle-specific. Mechanistic targeting can be distinguished based upon whether it is passive or active. Passive targeting utilizes the natural tendency of liposomes to distribute to cells of the reticuloendothelial system (RES) in organs which contain sinusoidal capillaries. Active targeting, on the other hand, involves alteration of the liposome by coupling the liposome to a specific ligand such as a monoclonal antibody, sugar, glycolipid, or protein, or by changing the composition or size of the liposome in order to achieve targeting to organs and cell types other than the naturally occurring sites of localization, specifically, cells that can become infected with a cytomegalovirus or interact with the proteins, peptides, and/or gene products of a cytomegalovirus, e.g., immune cells.

For any of the above approaches, the immune modulating polynucleotide construct, composition, or formulation is preferably applied to a site that will enhance the immune response. For example, the immunization may be intramuscular, intraperitoneal, enteral, parenteral, intranasal, intrapulmonary, or subcutaneous. In the gene delivery constructs of the instant invention, polynucleotide expression is directed from any suitable promoter (e.g., the human cytomegalovirus, simian virus 40, actin or adenovirus constitutive promoters; or the cytokine or metalloprotease promoters for activated synoviocyte specific expression).

In one example of the immune modifying peptide(s) or protein(s) include polynucleotides, constructs and/or mRNAs that express the one or more polynucleotides that express the one or more peptides or proteins, comprising, consisting of, or consisting essentially of an amino acid sequence selected from those sequences set forth in Table 1 or Table 2, or a subsequence, portion, homologue, variant or derivative thereof, that are designed to improve one or more of the stability and/or clearance in tissues, uptake and/or kinetics, cellular access by the peptide(s) or protein(s), translational, mRNA half-life, translation efficiency, immune evasion, protein production capacity, accessibility to circulation, peptide(s) or protein(s) half-life and/or presentation in the context of MHC on antigen presenting cells.

The present invention contemplates immunization for use in both active and passive immunization embodiments. Immunogenic compositions, proposed to be suitable for use as a vaccine, may be prepared most readily directly from immunogenic peptides, proteins, monomers, multimers and/or peptide-MHC complexes prepared in a manner disclosed herein. The antigenic material is generally processed to remove undesired contaminants, such as, small molecular weight molecules, incomplete proteins, or when manufactured in plant cells, plant components such as cell walls, plant proteins, and the like. Often, these immunizations are lyophilized for ease of transport and/or to increase shelf-life and can then be more readily dissolved in a desired vehicle, such as saline.

The preparation of immunizations (also referred to as vaccines) that contain the immunogenic proteins of the present invention as active ingredients is generally well understood in the art, as exemplified by United States Letters Patents 4,608,251; 4,601,903; 4,599,231; 4,599,230; 4,596,792; and 4.578,770, all incorporated herein by reference. Typically, such immunizations are prepared as injectables. The immunizations can be a liquid solution or suspension but may also be provided in a solid form suitable for solution in, or suspension in, liquid prior to injection may also be prepared. The preparation may also be emulsified. The active immunogenic ingredient is often mixed with excipients that are pharmaceutically acceptable and compatible with the active ingredient. Suitable excipients are, for example, water, saline, dextrose, glycerol, ethanol, buffers, or the like and combinations thereof. In addition, if desired, the immunization may contain minor amounts of auxiliary substances such as wetting or emulsifying agents, pH buffering agents, or adjuvants which enhance the effectiveness of the vaccines.

The immunization is/are administered in a manner compatible with the dosage formulation, and in such amount as will be therapeutically effective and immunogenic. The quantity to be administered depends on the subject to be treated, including, e.g., the capacity of the individual's immune system to synthesize antibodies, and the degree of protection desired. Precise amounts of active ingredient required to be administered depend on the judgment of the practitioner. However, suitable dosage ranges are of the order of several hundred micrograms active ingredient per vaccination. Suitable regimes for initial administration and booster shots are also variable but are typified by an initial administration followed by subsequent inoculations or other administrations.

The manner of application of the immunization may be varied widely. Any of the conventional methods for administration of a vaccine are applicable. These are believed to also include oral application on a solid physiologically acceptable base or in a physiologically acceptable dispersion, parenterally, by injection or the like. The dosage of the vaccine will depend on the route of administration and will vary according to the size of the host.

Various methods of achieving adjuvant effect for the vaccine includes use of agents such as aluminum hydroxide or phosphate (alum), commonly used as 0.05 to 0.1 percent solution in phosphate buffered saline, admixture with synthetic polymers of sugars (Carbopol) used as 0.25 percent solution, aggregation of the protein in the vaccine by heat treatment with temperatures ranging between 70° to 101° C. for 30 second to 2-minute periods respectively. Aggregation by reactivating with pepsin treated (Fab) antibodies to albumin, mixture with bacterial cells such as C. parvum or endotoxins or lipopolysaccharide components of gram-negative bacteria, emulsion in physiologically acceptable oil vehicles such as mannide mono-oleate (Aracel A) or emulsion with 20 percent solution of a perfluorocarbon (Fluosol-DA) used as a block substitute may also be employed.

In many instances, it will be desirable to have multiple administrations of the vaccine, usually not exceeding six to ten immunizations, more usually not exceeding four immunizations and preferably one or more, usually at least about three immunizations. The immunizations will normally be at from two to twelve-week intervals, more usually from three to five-week intervals. Periodic boosters at intervals of 1-5 years, usually three years, will be desirable to maintain protective levels of the antibodies. The course of the immunization may be followed by assays for antibodies for the supernatant antigens. The assays may be performed by labeling with conventional labels, such as radionuclides, enzymes, fluorescent agents, and the like. These techniques are well known and may be found in a wide variety of patents, such as Hudson and Cranage, Vaccine Protocols, 2003 Humana Press, relevant portions incorporated herein by reference.

Techniques and compositions for making useful dosage forms using the present invention are described in one or more of the following references: Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 2007; Goodman and Gilman, eds., The Pharmacological Basis of Therapeutics, Tenth Edition, McGraw Hill, 2001; Remington's Pharmaceutical Sciences, 20th Ed., Lippincott Williams & Wilkins., 2000, and updates thereto; Martindale, The Extra Pharmacopoeia, Thirty-Second Edition (The Pharmaceutical Press, London, 1999); all of which are incorporated by reference, and the like, relevant portions incorporated herein by reference.

Many suitable expression systems are commercially available, including, for example, the following: baculovirus expression (Reilly, P. R., et al., BACULOVIRUS EXPRESSION VECTORS: A LABORATORY MANUAL (1992); Beames, et al., Biotechniques 11:378 (1991); Pharmingen; Clontech, Palo Alto, Calif)), vaccinia expression systems (Earl, P. L., et al., “Expression of proteins in mammalian cells using vaccinia” In Current Protocols in Molecular Biology (F. M. Ausubel, et al. Eds.), Greene Publishing Associates & Wiley Interscience, New York (1991); Moss, B., et al., U.S. Pat. No. 5,135,855, issued Aug. 4, 1992), expression in bacteria (Ausubel, F. M., et al., CURRENT PROTOCOLS IN MOLECULAR BIOLOGY, John Wiley and Sons, Inc., Media Pa.; Clontech), expression in yeast (Rosenberg, S. and Tekamp-Olson, P., U.S. Pat. No. RE35,749, issued, Mar. 17, 1998, herein incorporated by reference; Shuster, J. R., U.S. Pat. No. 5,629,203, issued May 13, 1997, herein incorporated by reference; Gellissen, G., et al., Antonie Van Leeuwenhoek, 62(1-2):79-93 (1992); Romanos, M. A., et al., Yeast 8(6):423-488 (1992); Goeddel, D. V., Methods in Enzymology 185 (1990); Guthrie, C., and G. R. Fink, Methods in Enzymology 194 (1991)), expression in mammalian cells (Clontech; Gibco-BRL, Ground Island, N.Y.; e.g., Chinese hamster ovary (CHO) cell lines (Haynes, J., et al., Nuc. Acid. Res. 11:687-706 (1983); 1983, Lau, Y. F., et al., Mol. Cell. Biol. 4:1469-1475 (1984); Kaufman, R. J., “Selection and coamplification of heterologous genes in mammalian cells,” in Methods in Enzymology, vol. 185, pp 537-566. Academic Press, Inc., San Diego Calif (1991)), and expression in plant cells (plant cloning vectors, Clontech Laboratories, Inc., Palo-Alto, Calif., and Pharmacia LKB Biotechnology, Inc., Pistcataway, N.J.; Hood, E., et al., J. Bacteriol. 168:1291-1301 (1986); Nagel, R., et al., FEMS Microbiol. Lett. 67:325 (1990); An, et al., “Binary Vectors”, and others in Plant Molecular Biology Manual A3:1-19 (1988); Miki, B. L. A., et al., pp. 249-265, and others in Plant DNA Infectious Agents (Hohn, T., et al., eds.) Springer-Verlag, Wien, Austria, (1987); Plant Molecular Biology: Essential Techniques, P. G. Jones and J. M. Sutton, New York, J. Wiley, 1997; Miglani, Gurbachan Dictionary of Plant Genetics and Molecular Biology, New York, Food Products Press, 1998; Henry, R. J., Practical Applications of Plant Molecular Biology, New York, Chapman & Hall, 1997), relevant portion incorporated herein by reference.

As used herein, the term “effective amount” or “effective dose” refers to that amount of the peptide or protein T cell epitopes of the invention sufficient to induce immunity, to prevent and/or ameliorate an infection or to reduce at least one symptom of an infection and/or to enhance the efficacy of another dose of peptide or protein T cell epitopes. An effective dose may refer to the amount of peptide or protein T cell epitopes sufficient to delay or minimize the onset of an infection. An effective dose may also refer to the amount of peptide or protein T cell epitopes that provides a therapeutic benefit in the treatment or management of an infection. Further, an effective dose is the amount with respect to peptide or protein T cell epitopes of the invention alone, or in combination with other therapies, that provides a therapeutic benefit in the treatment or management of an infection. An effective dose may also be the amount sufficient to enhance a subject's (e.g., a human's) own immune response against a subsequent exposure to an infectious agent. Levels of immunity can be monitored, e.g., by measuring amounts of neutralizing secretory and/or serum antibodies, e.g., by plaque neutralization, complement fixation, enzyme-linked immunosorbent, or microneutralization assay. In the case of a vaccine, an “effective dose” is one that prevents disease and/or reduces the severity of symptoms. A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms, in this case, an infectious disease, and more particularly, a cytomegalovirus infection. The full prophylactic effect does not necessarily occur by administration of one dose, and may occur only after administration of a series of doses. Thus, a prophylactically effective amount may be administered in one or more administrations. Guidance can be found in the literature for appropriate dosages for given classes of pharmaceutical products. For example, for the given parameter, an effective amount will show an increase or decrease of at least 5%, 10%, 15%, 20%, 25%, 40%, 50%, 60%, 75%, 80%, 90%, or at least 100%. Efficacy can also be expressed as “-fold” increase or decrease. For example, a therapeutically effective amount can have at least a 1.2-fold, 1.5-fold, 2-fold, 5-fold, or more effect over a control. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins), relevant portions incorporated herein by reference.

As used herein, the term “immune stimulator” refers to a compound that enhances an immune response via the body's own chemical messengers (cytokines). These molecules comprise various cytokines, lymphokines and chemokines with immunostimulatory, immunopotentiating, and pro-inflammatory activities, such as interferons, interleukins (e.g., IL-1, IL-2, IL-3, IL-4, IL-12, IL-13); growth factors (e.g., granulocyte-macrophage (GM)-colony stimulating factor (CSF)); and other immunostimulatory molecules, such as macrophage inflammatory factor, Flt3 ligand, B7.1; B7.2, etc. The immune stimulator molecules can be administered in the same formulation as peptide or protein T cell epitopes s of the invention, or can be administered separately. Either the protein or an expression vector encoding the protein can be administered to produce an immunostimulatory effect.

As used herein, in certain embodiments, the term “protective immune response” or “protective response” refers to an immune response mediated by antibodies against an infectious agent, which is exhibited by a vertebrate (e.g., a human), which prevents or ameliorates an infection or reduces at least one symptom thereof. Peptide and protein T cell epitopes of the invention can stimulate the production of antibodies that, for example, neutralize infectious agents, blocks infectious agents from entering cells, blocks replication of said infectious agents, and/or protect host cells from infection and destruction. In other embodiments, the term can also refer to an immune response that is mediated by T-lymphocytes and/or other white blood cells against an infectious agent, exhibited by a vertebrate (e.g., a human), that prevents or ameliorates flavivirus infection or reduces at least one symptom thereof. Peptide and protein T cell epitopes of the invention can stimulate the T cell responses that, for example, neutralize infectious agents, kill virus infected cells, blocks infectious agents from entering cells, blocks replication of said infectious agents, and/or protect host cells from infection and destruction.

The terms “biological sample” or “sample” refer to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, and the like), sputum, tissue, cultured cells (e.g., primary cultures, explants, and transformed cells) stool, urine, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

The terms “virus” or “virus particle” are used according to its plain ordinary meaning within Virology and refers to a virion including the viral genome (e.g., DNA, RNA, single strand, double strand), viral capsid and associated proteins, and in the case of enveloped viruses (e.g., herpesvirus), an envelope including lipids and optionally components of host cell membranes, and/or viral proteins. In embodiments, the virus is a cytomegalovirus.

As used herein, a “cell” refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaryotic cells. Prokaryotic cells include but are not limited to bacteria. Eukaryotic cells include but are not limited to yeast cells and cells derived from plants and animals, for example mammalian, insect (e.g., Spodoptera) and human cells. Cells may be useful when they are naturally nonadherent or have been treated not to adhere to surfaces, for example by trypsinization.

As used herein, the term “contacting” is used in accordance with its plain ordinary meaning and refers to the process of allowing at least two distinct species to become sufficiently proximal to react, interact or physically touch. It should be appreciated, however, the resulting reaction product can be produced directly from a reaction between the added reagents or from an intermediate from one or more of the added reagents which can be produced in the reaction mixture. The term “contacting” may include allowing two species to react, interact, or physically touch, wherein the two species may be, for example, an amino acid sequence, protein, or peptide as provided herein and an immune cell, such as a T cell.

As used herein, a “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are valuable in a given situation and be able to analyze data based on comparisons to control values. Controls are also valuable for determining the significance of data. For example, if values for a given parameter are widely variant in controls, variation in test samples will not be considered as significant.

The term “modulator” refers to a composition that increases or decreases the level of a target molecule or the function of a target molecule or the physical state of the target of the molecule relative to the absence of the modulator.

The term “modulate” is used in accordance with its plain ordinary meaning and refers to the act of changing or varying one or more properties. “Modulation” refers to the process of changing or varying one or more properties. For example, as applied to the effects of a modulator on a target protein, to modulate means to change by increasing or decreasing a property or function of the target molecule or the amount of the target molecule.

The terms “associated” or “associated with” in the context of a substance or substance activity or function associated with a disease (e.g. a protein associated disease, a cancer (e.g., cancer, inflammatory disease, autoimmune disease, or infectious disease)) means that the disease (e.g. cancer, inflammatory disease, autoimmune disease, or infectious disease) is caused by (in whole or in part), or a symptom of the disease is caused by (in whole or in part) the substance or substance activity or function. As used herein, what is described as being associated with a disease, if a causative agent, could be a target for treatment of the disease.

The term “aberrant” as used herein refers to different from normal. When used to describe enzymatic activity or protein function, aberrant refers to activity or function that is greater or less than a normal control or the average of normal non-diseased control samples. Aberrant activity may refer to an amount of activity that results in a disease, wherein returning the aberrant activity to a normal or non-disease-associated amount (e.g., by administering a compound or using a method as described herein), results in reduction of the disease or one or more disease symptoms.

The terms “subject” or “subject in need thereof” refers to a living organism who is at risk of or prone to having a disease or condition, or who is suffering from a disease or condition that can be treated by administration of a composition or pharmaceutical composition as provided herein. Non-limiting examples include humans and other primates, but also includes non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered. The system described above is intended for use in any of the above vertebrate species, since the immune systems of all of these vertebrates operate similarly.

The terms “disease” or “condition” refer to a state of being or health status of a patient or subject capable of being treated with a compound, pharmaceutical composition, or method provided herein. In embodiments, a patient or subject is human. In embodiments, the disease is cytomegalovirus infection. In certain alternative embodiments, the disease is HCMV infection.

As used herein, “treatment” or “treating,” or “palliating” or “ameliorating” are used interchangeably herein. These terms refer to an approach for obtaining beneficial or desired results including but not limited to therapeutic benefit and/or a prophylactic benefit. By therapeutic benefit is meant eradication or amelioration of the underlying disorder being treated or the disorder resulting from viral infection. Also, a therapeutic benefit is achieved with the eradication or amelioration of one or more of the physiological symptoms associated with viral infection or the underlying disorder such that an improvement is observed in the patient, notwithstanding that the patient may still be afflicted with the underlying disorder or may still be infected. For prophylactic benefit, the compositions may be administered to a patient at risk of viral infection, of developing a particular disease, or to a patient reporting one or more of the physiological symptoms of a disease, even though a diagnosis of this disease may not have been made. Treatment includes preventing the infection or disease, that is, causing the clinical symptoms of the disease not to develop by administration of a protective composition prior to infection or the induction of the disease; suppressing the disease, that is, causing the clinical symptoms of the disease or infection not to develop by administration of a protective composition after the inductive event or infection but prior to the clinical appearance or reappearance of the disease; inhibiting the disease, that is, arresting the development of clinical symptoms by administration of a protective composition after their initial appearance; preventing re-occurring of the disease and/or relieving the disease, that is, causing the regression of clinical symptoms by administration of a protective composition after their initial appearance. “Treatment” can also refer to any of (i) the prevention of infection or reinfection, as in a traditional vaccine, (ii) the reduction or elimination of symptoms, and (iii) the substantial or complete elimination of the pathogen in question. Treatment may be affected prophylactically (prior to infection) or therapeutically (following infection).

In addition, in certain embodiments, “treatment,” “treat,” or “treating” refers to a method of reducing the effects of one or more symptoms of infection with a cytomegalovirus. Thus, in the disclosed method, treatment can refer to a 10%, 20%, 30%, 40%, 50%, 60%, 70%, 80%, 90%, or 100% reduction in the severity of an established infection, disease, condition, or symptom of the infection, disease or condition. For example, a method for treating a disease is considered to be a treatment if there is a 10% reduction in one or more symptoms of the disease in a subject as compared to a control. Thus, the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition and/or complete prevention of infection. Further, as used herein, references to decreasing, reducing, or inhibiting include a change of 10%, 20%, 30%, 40%, 50%, 60%, 70%, 80%, 90% or greater as compared to a control level and such terms can include but do not necessarily include complete elimination.

As used herein the terms “diagnose” or “diagnosing” refers to recognition of an infection, disease or condition by signs and symptoms. Diagnosing can refer to determination of whether a subject has an infection or disease. Diagnosis may refer to determination of the type of disease or condition a subject has or the type of virus the subject is infected with.

Diagnostic agents provided herein include any such agent, which are well-known in the relevant art. Among imaging agents are fluorescent and luminescent substances, including, but not limited to, a variety of organic or inorganic small molecules commonly referred to as “dyes,” “labels,” or “indicators.” Examples include fluorescein, rhodamine, acridine dyes, Alexa dyes, and cyanine dyes. Enzymes that may be used as imaging agents in accordance with the embodiments of the disclosure include, but are not limited to, horseradish peroxidase, alkaline phosphatase, acid phosphatase, glucose oxidase, 0-galactosidase, 0-glucuronidase or 0-lactamase. Such enzymes may be used in combination with a chromogen, a fluorogenic compound or a luminogenic compound to generate a detectable signal.

The peptide(s) or protein(s) of the present invention can also be used in binding assays including, but are not limited to, immunoassays such as competitive and non-competitive assay systems using techniques such as western blots, radioimmunoassays, ELISA (enzyme linked immunosorbent assay), “sandwich” immunoassays, Meso Scale Discovery (MSD, Gaithersburg, Md.), immunoprecipitation assays, ELISPOT, precipitin reactions, gel diffusion precipitin reactions, immunodiffusion assays, agglutination assays, complement-fixation assays, immunoradiometric assays, fluorescent immunoassays, and protein A immunoassays. Such assays are routine and well known in the art (see, e.g., Ausubel et al., eds, 1994, Current Protocols in Molecular Biology, Vol. 1, John Wiley & Sons, Inc., New York, relevant portions incorporated herein by reference).

Radioactive substances that may be used as imaging agents in accordance with the embodiments of the disclosure include, but are not limited to, ¹⁸F, ³²P, ³³P, ⁴⁵Ti, ⁴⁷Sc, ⁵²Fe, ⁵⁹Fe, ⁶²Cu, ⁶⁴Cu, ⁶⁷Cu, ⁶⁷Ga, ⁶⁸Ga, ⁷⁷As, ⁸⁶Y, ⁹⁰Y, ⁸⁹Sr, ⁸⁹Zr, ⁹⁴Tc, ⁹⁴Tc, ^{99m}Tc, ⁹⁹Mo, ¹⁰⁵Pd, ¹⁰⁵Rh, ¹¹¹Ag, ¹¹¹In, ¹²³I, ¹²⁴I, ¹²⁵I, ¹³¹I, ¹⁴²Pr, ¹⁴³Pr, ¹⁴⁹Pm, ¹⁵³Sm, ¹⁵⁴⁻¹⁵⁸¹Gd, ¹⁶¹Tb, ¹⁶⁶Dy, ¹⁶⁶Ho, ¹⁶⁹Er, ¹⁷⁵Lu, ¹⁷⁷Lu, ¹⁸⁶Re, ¹⁸⁸Re, ¹⁸⁹Re, ¹⁹⁴Ir, ¹⁹⁸Au, ¹⁹⁹Au, ²¹¹At, ²¹¹Pb, ²¹²Bi, ²¹²Pb, ²¹³Bi, ²²³Ra and ²²⁵Ac. Paramagnetic ions that may be used as additional imaging agents in accordance with the embodiments of the disclosure include, but are not limited to, ions of transition and lanthanide metals (e.g., metals having atomic numbers of 21-29, 42, 43, 44, or 57-71). These metals include ions of Cr, V, Mn, Fe, Co, Ni, Cu, La, Ce, Pr, Nd, Pm, Sm, Eu, Gd, Tb, Dy, Ho, Er, Tm, Yb and Lu.

When the imaging agent is a radioactive metal or paramagnetic ion, the agent may be reacted with another long-tailed reagent having a long tail with one or more chelating groups attached to the long tail for binding to these ions. The long tail may be a polymer such as a polylysine, polysaccharide, or other derivatized or derivatizable chain having pendant groups to which the metals or ions may be added for binding. Examples of chelating groups that may be used according to the disclosure include, but are not limited to, ethylenediaminetetraacetic acid (EDTA), diethylenetriaminepentaacetic acid (DTPA), DOTA, NOTA, NETA, TETA, porphyrins, polyamines, crown ethers, bis-thiosemicarbazones, polyoximes, and like groups.

The terms “dose” and “dosage” are used interchangeably herein. A dose refers to the amount of active ingredient given to an individual at each administration. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress. The term “dosage form” refers to the particular format of the pharmaceutical or pharmaceutical composition, and depends on the route of administration. For example, a dosage form can be in a liquid form for nebulization, e.g., for inhalants, in a tablet or liquid, e.g., for oral delivery, or a saline solution, e.g., for injection.

As used herein, the term “administering” means oral administration, administration as a suppository, topical contact, intravenous, intraperitoneal, intramuscular, intralesional, intrathecal, intranasal or subcutaneous administration, or the implantation of a slow-release device, e.g., a mini-osmotic pump, to a subject. Administration is by any route, including parenteral and transmucosal (e.g., buccal, sublingual, palatal, gingival, nasal, vaginal, rectal, or transdermal). Parenteral administration includes, e.g., intravenous, intramuscular, intra-arteriole, intradermal, subcutaneous, intraperitoneal, intraventricular, and intracranial. Other modes of delivery include, but are not limited to, the use of liposomal formulations, intravenous infusion, transdermal patches, etc. By “co-administer” it is meant that a composition described herein is administered at the same time, just prior to, or just after the administration of one or more additional therapies, for example cancer therapies such as chemotherapy, hormonal therapy, radiotherapy, or immunotherapy. The compounds of the invention can be administered alone or can be co-administered to the patient. Co-administration is meant to include simultaneous or sequential administration of the compounds individually or in combination (more than one compound). Thus, the preparations can also be combined, when desired, with other active substances (e.g., to reduce metabolic degradation). The compositions of the present invention can be delivered by transdermally, by a topical route, formulated as applicator sticks, solutions, suspensions, emulsions, gels, creams, ointments, pastes, jellies, paints, powders, and aerosols.

Formulations suitable for oral administration can consist of (a) liquid solutions, such as an effective amount of the antibodies provided herein suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes). Additionally, these carriers can function as immunostimulating agents (i.e., adjuvants).

The term “adjuvant” refers to a compound that when administered in conjunction with the compositions provided herein including embodiments thereof, augments the composition's immune response. Generally, adjuvants are non-toxic, have high-purity, are degradable, and are stable.

Adjuvants can augment an immune response by several mechanisms including lymphocyte recruitment, stimulation of B and/or T cells, and stimulation of macrophages. The adjuvant increases the titer of induced antibodies and/or the binding affinity of induced antibodies relative to the situation if the immunogen were used alone. A variety of adjuvants can be used in combination with the agents provided herein including embodiments thereof, to elicit an immune response. Preferred adjuvants augment the intrinsic response to an immunogen without causing conformational changes in the immunogen that affect the qualitative form of the response. Preferred adjuvants include aluminum hydroxide and aluminum phosphate, 3 De-O-acylated monophosphoryl lipid A (MPL™) (see GB 2220211 (RIBI ImmunoChem Research Inc., Hamilton, Montana, now part of Corixa). Stimulon™ QS-21 is a triterpene glycoside or saponin isolated from the bark of the Quillaja Saponaria Molina tree found in South America (see Kensil et al., in Vaccine Design: The Subunit and Adjuvant Approach (eds. Powell & Newman, Plenum Press, NY, 1995); U.S. Pat. No. 5,057,540), (Aquila BioPharmaceuticals, Framingham, MA). Other adjuvants are oil in water emulsions (such as squalene or peanut oil), optionally in combination with immune stimulants, such as monophosphoryl lipid A (see Stoute et al., N. Engl. J. Med. 336, 86-91 (1997)), pluronic polymers, and killed mycobacteria. Another adjuvant is CpG (WO 98/40100). Adjuvants can be administered as a component of a therapeutic composition with an active agent or can be administered separately, before, concurrently with, or after administration of the therapeutic agent.

Other adjuvants contemplated for the invention are saponin adjuvants, such as Stimulon™ (QS-21, Aquila, Framingham, MA) or particles generated therefrom such as ISCOMs (immunostimulating complexes) and ISCOMATRIX. Other adjuvants include RC-529, GM-CSF and Complete Freund's Adjuvant (CFA) and Incomplete Freund's Adjuvant (IFA). Other adjuvants include cytokines, such as interleukins (e.g., IL-1 a and 3 peptides, IL-2, IL-4, IL-6, IL-12, IL-13, and IL-15), macrophage colony stimulating factor (M-CSF), granulocyte-macrophage colony stimulating factor (GM-CSF), tumor necrosis factor (TNF), chemokines, such as MIP1α and β and RANTES. Another class of adjuvants is glycolipid analogues including N-glycosylamides, N-glycosylureas and N-glycosylcarbamates, each of which is substituted in the sugar residue by an amino acid, as immuno-modulators or adjuvants (see U.S. Pat. No. 4,855,283). Heat shock proteins, e.g., HSP70 and HSP90, may also be used as adjuvants.

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the compound of choice with a base, including, for example, liquid triglycerides, polyethylene glycols, and paraffin hydrocarbons.

Formulations suitable for parenteral administration, such as, for example, by intraarticular (in the joints), intravenous, intramuscular, intratumoral, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration, oral administration, and intravenous administration are the preferred methods of administration. The formulations of compounds can be presented in unit-dose or multi-dose sealed containers, such as ampules and vials.

Injection solutions and suspensions can be prepared from sterile powders, granules, and tablets of the kind previously described. Cells transduced by nucleic acids for ex vivo therapy can also be administered intravenously or parenterally as described above.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents.

The combined administration contemplates co-administration, using separate formulations or a single pharmaceutical formulation, and consecutive administration in either order, wherein preferably there is a time period while both (or all) active agents simultaneously exert their biological activities.

Effective doses of the compositions provided herein vary depending upon many different factors, including means of administration, target site, physiological state of the patient, whether the patient is human or an animal, other medications administered, and whether treatment is prophylactic or therapeutic. However, a person of ordinary skill in the art would immediately recognize appropriate and/or equivalent doses looking at dosages of approved compositions for treating and preventing cancer for guidance.

As used herein, the term “pharmaceutically acceptable” is used synonymously with “physiologically acceptable” and “pharmacologically acceptable”. A pharmaceutical composition will generally comprise agents for buffering and preservation in storage, and can include buffers and carriers for appropriate delivery, depending on the route of administration. As used herein, the terms “pharmaceutically acceptable” or “pharmacologically acceptable” refer to a material which is not biologically or otherwise undesirable, i.e., the material may be administered to an individual in a formulation or composition without causing any unacceptable biological effects or interacting in a deleterious manner with any of the components of the composition in which it is contained.

“Pharmaceutically acceptable excipient” and “pharmaceutically acceptable carrier” refer to a substance that aids the administration of an active agent to and absorption by a subject and can be included in the compositions of the present invention without causing a significant adverse toxicological effect on the patient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances, and the like, that do not deleteriously react with the compounds of the invention. One of skill in the art will recognize that other pharmaceutical excipients are useful in the present invention.

The term “pharmaceutically acceptable salt” refers to salts derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

The pharmaceutical preparation is optionally in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The unit dosage form can be of a frozen dispersion.

The compositions of the present invention may additionally include components to provide sustained release and/or comfort. Such components include high molecular weight, anionic mucomimetic polymers, gelling polysaccharides and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes. The compositions of the present invention can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995; as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). In embodiments, the formulations of the compositions of the present invention can be delivered by the use of liposomes which fuse with the cellular membrane or are endocytosed, i.e., by employing receptor ligands attached to the liposome, that bind to surface membrane protein receptors of the cell resulting in endocytosis. By using liposomes, particularly where the liposome surface carries receptor ligands specific for target cells, or are otherwise preferentially directed to a specific organ, one can focus the delivery of the compositions of the present invention into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989). The compositions of the present invention can also be delivered as nanoparticles.

The present invention describes methods utilizing and compositions comprising or expressing T cell epitopes, T cell epitope-containing peptides, and T cell epitope-containing proteins associated with binding to a subset of the naturally occurring MHC Class II and/or MHC Class I molecules within the human population. Compositions comprising or expressing one or more of the disclosed peptides (e.g., the amino acid sequences set forth in any one of Tables 1-2) or polynucleotides encoding the same, covering different HLA Class II and/or MHC Class I alleles, capable of generating a treatment acting broadly on a population level are disclosed herein. As the antigen repertoire of MHC Class I and MHC Class II alleles varies from one individual to another and from one ethnic population to another, it is challenging to provide vaccines or peptide or epitopes-based immunotherapies that can be offered to subjects of any geographic region in the world or provide sufficient protection against infection across a wide segment of the populations unless numerous epitopes or peptides are included (e.g., in a vaccine). Taking into consideration the need for a single vaccine formulation that can provide protection across populations, if it desirable to provide a treatment containing or expressing proteins, peptides or epitopes that will provide protection against infection amongst the majority of the worldwide population. Also, taking into consideration the enormous costs and risks in the clinical development of new treatments and the increasing demands from regulatory bodies to meet high standards for toxicity testing, dose justification, safety and efficacy trials, it is desirable to provide treatments containing or expressing as few peptides as possible, but at the same time to be able to treat the majority of subjects in a worldwide population with a single immunotherapy. Such a product should comprise as a first requirement an expression or inclusion of combination of epitopes or peptides that are able to bind the worldwide MHC Class I and/or MHC Class II allele repertoire, and the resulting peptide-MHC complexes should as a second requirement be recognized by the T cells of the subject so as to induce the desired immunological reactions.

It is an object of claims of the present invention to provide improved epitope or peptide combinations for modulating an immune response, for treating a subject for an infection or aberrant immune response, and for use in diagnostic methods and kits comprising such peptide combinations. It is another object of the invention to provide epitope or peptide combinations exhibiting very good HLA Class I and Class II coverage in a worldwide population and being immunologically potent in a worldwide population. It is another object of the invention to provide epitope or peptide combinations having good cross reactivity to other viral strains, including co-circulating strains (for example, mutants) of cytomegaloviruses, including HCMV, etc. It is another object of the invention to provide epitope or peptide combinations of a relatively small number of epitopes or peptides yet obtaining at least 70%, and more preferably around 90-100% donor coverage in a donor cohort representative of a worldwide population. In certain embodiments, this is achieved by selecting one or more immunodominant and/or immunoprevalent proteins (e.g., a HCMV protein) or subsequences, portions, homologues, variants or derivatives thereof for use in the methods and compositions of the present disclosure, wherein said immunodominant and/or immunoprevalent proteins or subsequences, portions, homologues, variants or derivatives thereof comprise two or more epitopes that are immunodominant and/or immunoprevalant. In some embodiments, the two or more epitopes comprise two to ten epitopes and/or polynucleotides encoding the same. Another object of the invention is to provide epitope combinations which are so immunologically potent that even at very low doses of epitopes, the percentage of responding donors can be retained at a very high level in a donor cohort representative of a worldwide population. Another object of the invention is to provide epitope combinations which have minor risk of inducing IgE-mediated adverse events. An additional object of the invention is to provide proteins, peptides, or nucleic acids containing or expressing epitopes or combinations of such proteins, peptides or nucleic acids which have a sufficient solubility profile for being formulated in a pharmaceutical product, preferably which have acceptable estimated in vivo stability. One further objective of the invention is to select epitopes for use in the compositions and methods described herein, based on one or both of their immunodominance or immunoprevalence. A still further object of the invention is to select such epitopes and epitopes combinations not only in accordance with those embodiments previously described, but also those epitopes and epitope combinations capable of eliciting a B cell response and T cell response (e.g., selecting one or more peptides for use in the methods and compositions described herein capable of generating a T cell and antibody response in a subject).

Provided herein are methods and compositions for diagnosing, treating, and immunizing against a cytomegalovirus, including methods and compositions of detecting an immune response or immune cells relevant to a cytomegalovirus infection. These methods and compositions include vaccines, diagnostics, therapies, reagents and kits, for modulating, eliciting, or detecting T cells responsive to one or more cytomegalovirus peptides or proteins. The proteins and peptides described herein comprise, consist of, or consist essentially of: one or more amino acid sequences selected from those sequences set forth in Table 1 or Table 2, or a subsequence, portion, homologue, variant or derivative thereof; a fusion protein comprising one or more amino acid sequences selected from those sequences set forth in Table 1 or Table 2; a pool of 2 or more peptides selected from the amino acid sequences set forth in Table 1 or Table 2, or a polynucleotide that encodes one or more peptides or proteins, comprising, consisting of, or consisting essentially of an amino acid sequence selected from those sequences set forth in Table 1 or Table 2, or a subsequence, portion, homologue, variant or derivative thereof. In certain preferred embodiments, the cytomegalovirus is one or more of HCMV or a variant thereof. Further description and embodiments of such methods and compositions are provided in the definitions provided herein, and a person skilled in the art will recognize that the methods and compositions can be embodied in numerous variations, changes, and substitutions or as may occur to or be understood by one skilled in the art without departing from the invention.

The present inventors recognized that defining a comprehensive set of epitope specificities is important for several reasons. First, it allows the determination of whether within different HCMV antigens certain regions are immunodominant. This will be important for vaccine design, so as to ensure that vaccine constructs include not only regions targeted by neutralizing antibodies but also include regions capable of delivering sufficient T cell help and are suitable targets of CD4+ T cell activity. Additionally, a comprehensive set of epitopes helps define the breadth of responses, in terms of the average number of different CD4+ and CD8+ T cell HCMV epitopes generally recognized by each individual.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of′. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically, and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

TABLE 1
Unique hCMV Epitopes

| | | | | No. of | Magnitude |
|---|---|---|---|---|---|
| S. | Peptide | Peptide | | subjects | of |
| No | sequence | length | ORF(s) | responding | response |
| 1 | NGIRWQYQELQYLVE | 15 | ORFL46W, | 2 | 222 |
| | | | ORFL46W.iORF1_(UL13), | | |
| | | | ORFL46W.iORF2 | | |
| 2 | RYNALTVRSRDSLLL | 15 | ORFL46W, | 2 | 204 |
| | | | ORFL46W.iORF1_(UL13), | | |
| | | | ORFL46W.iORF2 | | |
| 3 | RVRTWFVQRTTLWRR | 15 | ORFL46W, | 1 | 60 |
| | | | ORFL46W.iORF1_(UL13), | | |
| | | | ORFL46W.iORF2 | | |
| 4 | GLWVSSYLVRRPMTI | 15 | ORFL46W, | 2 | 890 |
| | | | ORFL46W.iORF1_(UL13), | | |
| | | | ORFL46W.iORF2 | | |
| 5 | QGATYQLSIVRQAMQ | 15 | ORFL46W.iORF1_(UL13) | 1 | 650 |
| 6 | GAGLRQLRQQLTVRW | 15 | ORFL46W.iORF2 | 1 | 20 |
| 7 | MRTVPVTKLYTSRMV | 15 | ORFL49W_(UL16)(UL16P?) | 1 | 5677 |
| 8 | AITLFFFLLALRIPQ | 15 | ORFL49W.iORF1 | 1 | 207 |
| 9 | ALFTHFVGRPRHCRL | 15 | ORFL50W_(UL17) | 1 | 57 |
| 10 | MLGIRAMLVMLDYYW | 15 | ORFL53W_(UL20) | 1 | 350 |
| 11 | PSVRMDFRARRPLRR | 15 | ORFL55C_(UL21A) | 3 | 1740 |
| 12 | ARRLWILSLLAVTLT | 15 | ORFL57W_(UL22A), | 1 | 100 |
| | | | ORFL57W.iORF1 | | |
| 13 | LLAVTLTVALAAPSQ | 15 | ORFL57W_(UL22A) | 2 | 6420 |
| 14 | KDRCLVIRRRWRLVR | 15 | ORFL64C_(UL23) | 1 | 60 |
| 15 | FVAESITEFLNIGLR | 15 | ORFL64C_(UL23) | 1 | 530 |
| 16 | HENGIYYGTRSMRKL | 15 | ORFL64C_(UL23), | 1 | 827 |
| | | | ORFL64C.iORF1 | | |
| 17 | FCRRFFFPDRPDFFL | 15 | ORFL65C | 1 | 107 |
| 18 | AEDSVFTSTRARSAT | 15 | ORFL70W_(UL25) | 1 | 490 |
| 19 | KFVLQDFDVQHLRRL | 15 | ORFL70W_(UL25) | 1 | 40 |
| 20 | IINYYYVAQKKARHM | 15 | ORFL70W_(UL25) | 1 | 163 |
| 21 | ALALHFLTSRKGVTD | 15 | ORFL70W_(UL25) | 1 | 40 |
| 22 | LMITHFORTIRVLRC | 15 | ORFL70W_(UL25) | 3 | 2421 |
| 23 | DFLRVVRQQDAFICT | 15 | ORFL70W_(UL25) | 2 | 174 |
| 24 | ICVARLQAQPSSRHI | 15 | ORFL70W_(UL25) | 1 | 37 |
| 25 | GVSSVTLLKIFSQVP | 15 | ORFL70W_(UL25) | 2 | 220 |
| 26 | VLATLAAVRTRRRSV | 15 | ORFL71C, ORFL71C.iORF1 | 2 | 340 |
| | | | (UL24) | | |
| 27 | EAYVRINAGQVLPVV | 15 | ORFL71C, ORFL71C.iORF1 | 1 | 1853 |
| | | | (UL24) | | |
| 28 | LHCMRYLTSSLVKRY | 15 | ORFL71C | 1 | 150 |
| 29 | KRYFRPLLRAWSLGL | 15 | ORFL71C, ORFL71C.iORF1 | 4 | 1388 |
| | | | (UL24) | | |
| 30 | HLLRNIKTAFGMRVL | 15 | ORFL71C, ORFL71C.iORF1 | 2 | 1093 |
| | | | (UL24) | | |
| 31 | ARNLMEFARVGLRAV | 15 | ORFL71C.iORF1 (UL24) | 1 | 1450 |
| 32 | TGLVLLLLLLVVRLL | 15 | ORFL73C | 1 | 1440 |
| 33 | MLFRPTISNSIPRCR | 15 | ORFL76C | 1 | 47 |
| 34 | LRIIRLLRASIRHEY | 15 | ORFL79C_(UL27) | 1 | 810 |
| 35 | RAHIQKFERLHVRRF | 15 | ORFL79C_(UL27) | 1 | 2523 |
| 36 | SLQFIGLQRRDVVAL | 15 | ORFL92C_(UL32) | 1 | 83 |
| 37 | RDVVALVNFLRHLTQ | 15 | ORFL92C_(UL32) | 1 | 1710 |
| 38 | RRTVLFNELMLWLGY | 15 | ORFL92C_(UL32) | 1 | 180 |
| 39 | VNAVNKLVYTGRLIM | 15 | ORFL92C_(UL32) | 1 | 340 |
| 40 | KELRMCLSFDSNYCR | 15 | ORFL92C_(UL32) | 1 | 127 |
| 41 | GMKTVAFDLSSPQKS | 15 | ORFL92C.iORF1 | 1 | 193 |
| 42 | NAIVLITQLLTNRVL | 15 | ORFL93W_(UL33) | 1 | 37 |
| 43 | STNFLTLTVLPFIVL | 15 | ORFL93W_(UL33) | 1 | 63 |
| 44 | VLPFIVLSNQWLLPA | 15 | ORFL93W_(UL33) | 1 | 247 |
| 45 | FATVALIAADRYRVL | 15 | ORFL93W_(UL33) | 4 | 2310 |
| 46 | SYRSTYIILLLTWFA | 15 | ORFL93W_(UL33) | 3 | 3990 |
| 47 | LTLRRTIGTLSRLVP | 15 | ORFL93W_(UL33) | 1 | 163 |
| 48 | RRRMVSVTLFSPYSV | 15 | ORFL98W.iORF1, | 1 | 53 |
| | | | ORFL98W.iORF2 | | |
| 49 | GRLMEVRQRNGRLRR | 15 | ORFL100C | 1 | 30 |
| 50 | WPERCFIQLRSRSAL | 15 | ORFL101C, | 3 | 313 |
| | | | ORFL101C.iORF1_(UL36) | | |
| 51 | GPGFMRYQLIVLIGQ | 15 | ORFL101C, | 1 | 3167 |
| | | | ORFL101C.iORF1_(UL36) | | |
| 52 | IQTMELMIRTVPRIT | 15 | ORFL101C, | 2 | 384 |
| | | | ORFL101C.iORF1_(UL36) | | |
| 53 | EFLVRQYVLVDTFGV | 15 | ORFL101C | 2 | 104 |
| 54 | RREAIVRLEKTPTCQ | 15 | ORFL101C, | 2 | 407 |
| | | | ORFL101C.iORF1_(UL36) | | |
| 55 | RRRFKVCDVGRRHII | 15 | ORFL101C, | 1 | 63 |
| | | | ORFL101C.iORF1_(UL36) | | |
| 56 | RHRFLWQRRRRARLL | 15 | ORFL103C_(vMIA), | 1 | 1000 |
| | | | ORFL104C_(UL37) | | |
| 57 | GSFSSFYSQIARSLG | 15 | ORFL105C_(UL40) | 1 | 363 |
| 58 | FLKKMLLCALKGRAS | 15 | ORFL115C_(UL45), | 1 | 930 |
| | | | ORFL115C.iORF1 | | |
| 59 | MPVQRLTVNVARCVF | 15 | ORFL115C_(UL45) | 1 | 20 |
| 60 | KFIFELYRLPRLSIA | 15 | ORFL115C_(UL45) | 1 | 173 |
| 61 | ASKIKMLETRVTLAL | 15 | ORFL116W_(UL47) | 1 | 140 |
| 62 | ATMLSKYTRMSSLFN | 15 | ORFL127C_(UL48A) | 2 | 650 |
| 63 | AFKLDLLRMIAVSRT | 15 | ORFL127C_(UL48A) | 2 | 477 |
| 64 | MLFFQRYAPAFVTGY | 15 | ORFL143C_(UL54) | 1 | 57 |
| 65 | DLKYILTRLEYLYKV | 15 | ORFL143C_(UL54) | 1 | 30 |
| 66 | DPSYVREHGVPIHAD | 15 | ORFL143C_(UL54) | 1 | 123 |
| 67 | TDLIRFERNIVCTSM | 15 | ORFL145C_(UL55) | 3 | 273 |
| 68 | EGIMVVYKRNIVAHT | 15 | ORFL145C_(UL55) | 1 | 637 |
| 69 | HTFKVRVYQKVLTFR | 15 | ORFL145C_(UL55) | 1 | 423 |
| 70 | YQKVLTFRRSYAYIH | 15 | ORFL145C_(UL55) | 1 | 40 |
| 71 | RRSYAYIHTTYLLGS | 15 | ORFL145C_(UL55) | 5 | 2730 |
| 72 | QLMPDDYSNTHSTRY | 15 | ORFL145C_(UL55) | 5 | 45517 |
| 73 | NLNCMVTITTARSKY | 15 | ORFL145C_(UL55) | 2 | 4810 |
| 74 | NADKFFIFPNYTIVS | 15 | ORFL145C_(UL55) | 4 | 5120 |
| 75 | GLVVFWQGIKQKSLV | 15 | ORFL145C_(UL55) | 1 | 53 |
| 76 | QLQFTYDTLRGYINR | 15 | ORFL145C_(UL55) | 3 | 2236 |
| 77 | LRGYINRALAQIAEA | 15 | ORFL145C_(UL55) | 3 | 30337 |
| 78 | KELSKINPSAILSAI | 15 | ORFL145C_(UL55) | 3 | 18557 |
| 79 | AILSAIYNKPIAARF | 15 | ORFL145C_(UL55) | 5 | 11264 |
| 80 | ASCVTINQTSVKVLR | 15 | ORFL145C_(UL55) | 5 | 8941 |
| 81 | YLFKRMIDLSSISTV | 15 | ORFL145C_(UL55) | 1 | 67 |
| 82 | EQAYQMLLALARLDA | 15 | ORFL145C_(UL55) | 4 | 21807 |
| 83 | LLDRLRHRKNGYRHL | 15 | ORFL145C.iORF1 | 3 | 12544 |
| 84 | QILWTDGLARRTRDR | 15 | ORFL145C.iORF2 | 1 | 37 |
| 85 | RVGITIQQLNVYHQL | 15 | ORFL146C_(UL56) | 1 | 963 |
| 86 | TMRSVFEMQRIRHGA | 15 | ORFL147C | 1 | 67 |
| 87 | NIFLVGFYLLVPYLG | 15 | ORFL147C | 1 | 1633 |
| 88 | SLLILVVLLLIYRCC | 15 | ORFL159W | 1 | 23 |
| 89 | LSYMKYHHLHGLPVN | 15 | ORFL161C_(UL69) | 3 | 879 |
| 90 | VELCLGAGAGHVVVV | 15 | ORFL162W | 1 | 383 |
| 91 | RSSWRASCVEVPKKP | 15 | ORFL165W | 1 | 370 |
| 92 | MQKYFSLDNFLHDYV | 15 | UL70 | 2 | 20343 |
| 93 | QTIYFLGLTALLLRY | 15 | ORFL181C_(UL74) | 1 | 583 |
| 94 | SFYLVNAMSRNLFRV | 15 | ORFL181C_(UL74) | 1 | 43 |
| 95 | TMRKLKRKQALVKEQ | 15 | ORFL181C_(UL74) | 1 | 1563 |
| 96 | TAVSEFMKNTHVLIR | 15 | ORFL181C.iORF1 | 1 | 747 |
| 97 | WREDVLMDRVRKRYL | 15 | ORFL189W_(UL77) | 1 | 67 |
| 98 | IKMWFLLGAPMIAVL | 15 | ORFL196W_(UL78), | 3 | 2867 |
| | | | ORFL196W.iORF1 | | |
| 99 | LFIIAFFSREPTKDL | 15 | ORFL196W_(UL78) | 1 | 190 |
| 100 | PKSFTLTRIHPEYIV | 15 | ORFL202C_(UL82/pp71) | 2 | 340 |
| 101 | PEYIVQIQNAFETNQ | 15 | ORFL202C_(UL82/pp71) | 4 | 527 |
| 102 | GALTLVIPSWHVFAS | 15 | ORFL202C_(UL82/pp71) | 2 | 190 |
| 103 | CRSATSLVGNTNADV | 15 | ORFL203W | 1 | 30 |
| 104 | SSCAHTTCRSATSLV | 15 | ORFL203W | 2 | 104 |
| 105 | SWLGQMLRPVGLCTL | 15 | ORFL204W | 1 | 43 |
| 106 | QTGIHVRVSQPSLIL | 15 | ORFL205C_(UL83/pp65), | 11 | 33563 |
| | | | ORFL205C.iORF1 | | |
| 107 | MSIYVYALPLKMLNI | 15 | ORFL205C_(UL83/pp65) | 1 | 83 |
| 108 | PLKMLNIPSINVHHY | 15 | ORFL205C_(UL83/pp65) | 7 | 2920 |
| 109 | ATKMQVIGDQYVKVY | 15 | ORFL205C_(UL83/pp65), | 2 | 4637 |
| | | | ORFL205C.iORF1 | | |
| 110 | PKNMIIKPGKISHIM | 15 | ORFL205C_(UL83/pp65), | 5 | 538 |
| | | | ORFL205C.iORF1 | | |
| 111 | PGKISHIMLDVAFTS | 15 | ORFL205C_(UL83/pp65), | 9 | 2386 |
| | | | ORFL205C.iORF1 | | |
| 112 | MNGQQIFLEVQAIRE | 15 | ORFL205C_(UL83/pp65), | 6 | 3990 |
| | | | ORFL205C.iORF1 | | |
| 113 | ELRQYDPVAALFFFD | 15 | ORFL205C_(UL83/pp65) | 8 | 1106 |
| 114 | GILARNLVPMVATVQ | 15 | ORFL205C_(UL83/pp65), | 11 | 34633 |
| | | | ORFL205C.iORF1 | | |
| 115 | ALFFFDIDLLLQRGP | 15 | ORFL205C.iORF1 | 2 | 73 |
| 116 | RVTGLVFSVVFSVSL | 15 | ORFL206W | 4 | 6380 |
| 117 | LTWCVIADRQPRFSV | 15 | ORFL206W | 3 | 240 |
| 118 | RPKRRVVAPFRVAAA | 15 | ORFL207W | 2 | 283 |
| 119 | APFRVAAAGETPLGR | 15 | ORFL207W | 6 | 1089 |
| 120 | IPQRLHLIKHYQLGL | 15 | ORFL209C_(UL85) | 1 | 437 |
| 121 | IVPMPLALEINQRLL | 15 | ORFL209C_(UL85) | 1 | 283 |
| 122 | LASELTMTYVRKLAL | 15 | ORFL209C_(UL85) | 1 | 37 |
| 123 | HSILADFNSYKAHLT | 15 | ORFL212C_(UL86) Major Capsid | 1 | 27 |
| | | | Protein | | |
| 124 | FHELRTWEIMEHMRL | 15 | ORFL212C_(UL86) Major Capsid | 1 | 7343 |
| | | | Protein | | |
| 125 | PQLLFHYRNLVAVLR | 15 | ORFL212C_(UL86) Major Capsid | 1 | 60 |
| | | | Protein | | |
| 126 | RNLVAVLRLVTRISA | 15 | ORFL212C_(UL86) Major Capsid | 1 | 20 |
| | | | Protein | | |
| 127 | LFLAVQFVGEHVKVL | 15 | ORFL212C_(UL86) Major Capsid | 1 | 53 |
| | | | Protein | | |
| 128 | VRVQDLFRVFPMNVY | 15 | ORFL212C_(UL86) Major Capsid | 1 | 43 |
| | | | Protein | | |
| 129 | LGYNSKFYSPCAQYF | 15 | ORFL212C_(UL86) Major Capsid | 1 | 20 |
| | | | Protein | | |
| 130 | TQEALPILSTTTLAL | 15 | ORFL212C_(UL86) Major Capsid | 1 | 407 |
| | | | Protein | | |
| 131 | PFTVLRLSYAYRIFA | 15 | ORFL229W_(UL98) | 1 | 33 |
| 132 | AREFLLSHDAALFRA | 15 | ORFL229W_(UL98) | 1 | 23 |
| 133 | MLIQQYVLSQYYIKK | 15 | ORFL229W_(UL98) | 1 | 20 |
| 134 | RLGTAATQIQKQTLY | 15 | ORFL233C | 1 | 30 |
| 135 | KTQIFNKLFTNRISV | 15 | ORFL236C | 1 | 1587 |
| 136 | VRSLAVDAQHAAKRV | 15 | ORFL238W | 1 | 53 |
| 137 | LEERDEWVRSLAVDA | 15 | ORFL238W | 1 | 47 |
| 138 | AAITVVPVITQSRLA | 15 | ORFL245C | 3 | 450 |
| 139 | PWYPITQARTLELTP | 15 | ORFL246C | 2 | 996 |
| 140 | MSTKRSTVPWYPITQ | 15 | ORFL246C | 3 | 477 |
| 141 | LRVTFHRVKPTLQRE | 15 | ORFL248W.iORF1 | 2 | 830 |
| 142 | SGRVILWTTLRLCIL | 15 | ORFL249C | 1 | 20 |
| 143 | VVRKYWTFTNPNRIL | 15 | ORFL251W, ORFL252W, | 3 | 16876 |
| | | | ORFL253W_(UL112), | | |
| | | | ORFL253W.iORF1, | | |
| | | | ORFL253W.iORF2 | | |
| 144 | TFDVRQFVFDNARLV | 15 | ORFL251W, ORFL252W, | 6 | 13736 |
| | | | ORFL253W_(UL112), | | |
| | | | ORFL253W.iORF1, | | |
| | | | ORFL253W.iORF2 | | |
| 145 | VRGGIVENKSVSSVV | 15 | ORFL251W, ORFL252W, | 5 | 2691 |
| | | | ORFL253W_(UL112), | | |
| | | | ORFL253W.iORF1, | | |
| | | | ORFL253W.iORF2 | | |
| 146 | GNLQVTYVRHYLKNH | 15 | ORFL253W_(UL112), | 4 | 655 |
| | | | ORFL253W.iORF1, | | |
| | | | ORFL253W.iORF2 | | |
| 147 | AVAFLNYSSSSSAVS | 15 | ORFL253W_(UL112), | 3 | 2151 |
| | | | ORFL253W.iORF1, | | |
| | | | ORFL253W.iORF2 | | |
| 148 | AGLMMMRRMRRAPA | 15 | ORFL253W_(UL112) | 1 | 490 |
| | E | | | | |
| 149 | CDLPLVSSRLLPETS | 15 | ORFL253W_(UL112) | 1 | 123 |
| 150 | CEIKPYVVNPVVATA | 15 | ORFL253W_(UL112) | 3 | 2583 |
| 151 | DPLLRLSQVAGSGRR | 15 | ORFL253W_(UL112) | 2 | 2067 |
| 152 | LPLCSTARLRLAPRR | 15 | ORFL253W.iORF3 | 1 | 467 |
| 153 | RATGNFRSTSLYAAV | 15 | ORFL253W.iORF3 | 3 | 4220 |
| 154 | RCCTLRFRRRCRARC | 15 | ORFL253W.iORF4 | 2 | 713 |
| 155 | MSATRHHRCCTLRFR | 15 | ORFL253W.iORF4 | 1 | 277 |
| 156 | RVFCLSADWIRFLSL | 15 | ORFL254C_(UL114) | 2 | 846 |
| 157 | HLGWQTLSNHVIRRL | 15 | ORFL254C_(UL114) | 1 | 127 |
| 158 | TVVRLHVQIAGRSFT | 15 | ORFL258C_(UL119) | 1 | 213 |
| 159 | SCTHPYVISLVTPLT | 15 | ORFL260C_(UL121) | 1 | 80 |
| 160 | ISLVTPLTINATLRL | 15 | ORFL260C_(UL121) | 1 | 317 |
| 161 | CRVDADLGLLYAVCL | 15 | ORFL260C_(UL121) | 1 | 673 |
| 162 | VCLILSFSIVTAALW | 15 | ORFL260C_(UL121) | 1 | 43 |
| 163 | MFFLAIRDHDTAGGI | 15 | ORFL261W | 1 | 1637 |
| 164 | LQTMLRKEVNSQLSL | 15 | ORFL264C_(UL123) IE1, | 2 | 1606 |
| | | | ORFL265C_(UL122) IE2 | | |
| 165 | LVKQIKVRVDMVRHR | 15 | ORFL264C_(UL123) IE1 | 3 | 580 |
| 166 | RVDMVRHRIKEHMLK | 15 | ORFL264C_(UL123) IE1 | 2 | 250 |
| 167 | LRRKMMYMCYRNIEF | 15 | ORFL264C_(UL123) IE1 | 6 | 1697 |
| 168 | CSPDEIMSYAQKIFK | 15 | ORFL264C_(UL123) IE1 | 2 | 194 |
| 169 | EERDKVLTHIDHIFM | 15 | ORFL264C_(UL123) IE1 | 2 | 107 |
| 170 | VLCCYVLEETSVMLA | 15 | ORFL264C_(UL123) IE1 | 1 | 93 |
| 171 | ITKPEVISVMKRRIE | 15 | ORFL264C_(UL123) IE1 | 1 | 1600 |
| 172 | FAQYILGADPLRVCS | 15 | ORFL264C_(UL123) IE1 | 1 | 30 |
| 173 | EAIVAYTLATAGASS | 15 | ORFL264C_(UL123) IE1 | 3 | 19333 |
| 174 | TTRPFKVIIKPPVPP | 15 | ORFL265C_(UL122) IE2 | 2 | 424 |
| 175 | NKGIQIIYTRNHEVK | 15 | ORFL265C_(UL122) IE2, | 7 | 3794 |
| | | | ORFL265C.iORF1, | | |
| | | | ORFL265C.iORF2, | | |
| | | | ORFL265C.iORF3, | | |
| 176 | LGSMCNLALSTPFLM | 15 | ORFL265C_(UL122) IE2, | 4 | 668 |
| | | | ORFL265C.iORF1, | | |
| | | | ORFL265C.iORF2 | | |
| 177 | STPFLMEHTMPVTHP | 15 | ORFL265C_(UL122) IE2, | 4 | 740 |
| | | | ORFL265C.iORF1, | | |
| | | | ORFL265C.iORF2, | | |
| | | | ORFL265C.iORF3, | | |
| 178 | YRNMIIHAATPVDLL | 15 | ORFL265C.iORF3 | 2 | 130 |
| 179 | VMVRIFSTNQGGFML | 15 | ORFL265C.iORF3 | 2 | 3560 |
| 180 | VVVGIVLCLSLASTV | 15 | ORFL266W_(UL124) | 1 | 1417 |
| 181 | SPVAAELPHPSPAPM | 15 | ORFL267C | 2 | 166 |
| 182 | SYLAVHLRISHRYYH | 15 | ORFL269C | 1 | 290 |
| 183 | IAITMVMRFWQYING | 15 | ORFL270C | 3 | 163 |
| 184 | TALWLLLGHSRVPRV | 15 | UL128 | 1 | 177 |
| 185 | AEIRGIVTTMTHSLT | 15 | ORFL271C_(UL128_truncated) | 1 | 1713 |
| 186 | NPLYLEADGRIRCGK | 15 | ORFL271C_(UL128_truncated), | 2 | 884 |
| | | | UL128 strain GSV9 | | |
| 187 | LHRRAAVSGRRSLLQ | 15 | ORFL271C.iORF1 | 1 | 87 |
| 188 | MLRLLFTLVLLALYG | 15 | ORFL278C_(UL148) | 3 | 4593 |
| 189 | HVRLLSYRGDPLVFK | 15 | ORFL278C_(UL148) | 3 | 333 |
| 190 | VVRFALYLETLSRIV | 15 | ORFL278C_(UL148) | 2 | 123 |
| 191 | FYMNWTLRRSQTHYL | 15 | ORFL278C_(UL148) | 6 | 16883 |
| 192 | QVEILKPRGVRHRAI | 15 | ORFL278C_(UL148) | 9 | 3468 |
| 193 | FCVYRYNARLTRGYV | 15 | ORFL278C_(UL148) | 3 | 700 |
| 194 | TRGYVRYTLSPKARL | 15 | ORFL278C_(UL148) | 7 | 9337 |
| 195 | SLDRFIVQYLNTLLI | 15 | ORFL278C_(UL148) | 8 | 23368 |
| 196 | PTWSTTVNAHNSFLH | 15 | ORFL278C.iORF1 | 1 | 47 |
| 197 | DRLSTLAATMCMFDY | 15 | ORFL279C | 1 | 53 |
| 198 | LFYRAVALGTLSALV | 15 | ORFL280C_(UL147A) | 3 | 2633 |
| 199 | SSIFTSTHRGVIVAP | 15 | ORFL283W | 1 | 27 |
| 200 | LSVRYLSLTAYMLLA | 15 | ORFL284C_(UL147) | 1 | 1200 |
| 201 | TAYKAFLWKYAKKLN | 15 | ORFL284C_(UL147) | 1 | 503 |
| 202 | WKYAKKLNYHYFRLR | 15 | ORFL284C_(UL147) | 1 | 237 |
| 203 | VYLWYVRRQLVAFCL | 15 | ORFL318C_(UL148A) | 3 | 253 |
| 204 | FPSARDIPKQLPEQP | 15 | ORFL320W | 1 | 27 |
| 205 | VVAYVILERLWLAAR | 15 | ORFL321W.iORF1 | 1 | 23 |
| 206 | IRRWWISVAIVIFIG | 15 | ORFL321W.iORF2, | 3 | 10480 |
| | | | ORFL321W.iORF3_(UL148D) | | |
| 207 | RWQFAVCAASKTATR | 15 | ORFL322W | 1 | 50 |
| 208 | PQRLLLTALAIWQRT | 15 | ORFL324C_(UL150) | 1 | 983 |
| 209 | PWWRRLRVKRPKFPS | 15 | ORFS326C, | 1 | 240 |
| | | | ORFS326C.iORF1_(US1) | | |
| 210 | LWYLGDYGAILKIYF | 15 | ORFS337C_(US10) | 1 | 40 |
| 211 | LFCGACVITRSLLLI | 15 | ORFS337C_(US10) | 1 | 487 |
| 212 | MNLVMLILALWAPVA | 15 | ORFS338C_(US11) | 1 | 553 |
| 213 | VSEYRVEYSEARCVL | 15 | ORFS338C_(US11) | 1 | 263 |
| 214 | MLVVTVFDTTRLFEI | 15 | ORFS345C_(US17) | 1 | 840 |
| 215 | VCAFCWLVLPHRLEQ | 15 | ORFS351C_(US21) | 1 | 1960 |
| 216 | VSVLYFMPSEPGSAH | 15 | ORFS351C.iORF2 | 1 | 177 |
| 217 | VFQKTLSMLQGLYLR | 15 | ORFS352C_(US22) | 2 | 327 |
| 218 | GLYLRQYDPPALRTY | 15 | ORFS352C_(US22) | 2 | 633 |
| 219 | WFLVMREQAAIPQIY | 15 | ORFS352C_(US22) | 4 | 1100 |
| 220 | QIYARSLAADYLCCD | 15 | ORFS352C_(US22) | 1 | 33 |
| 221 | DFRDLLNFIRQRLCC | 15 | ORFS352C_(US22) | 1 | 30 |
| 222 | PSQEILLLCARHLDE | 15 | ORFS353C_(US23) | 3 | 110 |
| 223 | TDCWPFEVAPAARLA | 15 | ORFS353C_(US23) | 2 | 1637 |
| 224 | LFRAGLMKVYVRRRY | 15 | ORFS353C_(US23) | 1 | 870 |
| 225 | VVFMGRFSRVYAYDT | 15 | ORFS355C_(US24) | 1 | 70 |
| 226 | EKYMVLVSHNLDELA | 15 | ORFS355C_(US24) | 1 | 20 |
| 227 | PRLHCLVTTRSSTRE | 15 | ORFS355C.iORF1 | 1 | 1277 |
| 228 | LRYKWLIRKDRFIVR | 15 | ORFS361C_(US26) | 1 | 3247 |
| 229 | TNIMLQVSNVTNHTL | 15 | ORFS363W_(US27) | 1 | 58 |
| 230 | IVVGLPFFLEYAKHH | 15 | ORFS363W_(US27) | 2 | 1250 |
| 231 | YNRMVRFIINYVGKW | 15 | ORFS363W_(US27) | 1 | 23 |
| 232 | ITFCLYVGQFLAYVR | 15 | ORFS363W_(US27) | 1 | 27 |
| 233 | HDPLGLTRFIMRQLM | 15 | ORFS370W_(US33A) | 1 | 473 |
| 234 | FIMRQLMMYPLVLPF | 15 | ORFS370W_(US33A) | 1 | 530 |
| 235 | GLVYRELHDFYGYLQ | 15 | ORFS371W_(US34) | 1 | 963 |

TABLE 2
CMV SPECIFIC CLASS II EPITOPES

| S. | | Peptide | | |
|---|---|---|---|---|
| No | Peptide sequence | length | ORF | Antigen Name from IEDB |
| 1 | HINSHSQCYSSYSRVIA | 17 | ORFL145C_(UL55) | glycoprotein B |
| 2 | SRVIAGTVFVAYHRD | 15 | ORFL145C_(UL55) | glycoprotein B |
| 3 | CMVTITTARSKYPYH | 15 | ORFL145C_(UL55) | glycoprotein B |
| 4 | VFETTGGLVVFWQGI | 15 | ORFL145C_(UL55) | glycoprotein B |
| 5 | MQLIPDDYSNTHSTRYVTVK | 20 | ORFL145C_(UL55) | glycoprotein B |
| 6 | LPLKMLNIPSINVH | 14 | ORFL205C_(UL83/pp65) | 65 kDa lower matrix phosphoprotein |
| 7 | PQYSEHPTFTSQYRIQ | 16 | ORFL205C_(UL83/pp65) | 65 kDa lower matrix phosphoprotein |
| 8 | FTSQYRIQGKLEYRHT | 16 | ORFL205C_(UL83/pp65) | 65 kDa lower matrix phosphoprotein |
| 9 | PPWQAGILARNLVPMV | 16 | ORFL205C_(UL83/pp65) | 65 kDa lower matrix phosphoprotein |
| 10 | KYQEFFWDANDIYRIF | 16 | ORFL205C_(UL83/pp65) | 65 kDa lower matrix phosphoprotein |
| 11 | GPISGHVLKAVFSRG | 15 | ORFL205C_(UL83/pp65) | 65 kDa lower matrix phosphoprotein |
| 12 | LLQTGIHVRVSQPSL | 15 | ORFL205C_(UL83/pp65) | 65 kDa lower matrix phosphoprotein |
| 13 | IYVYALPLKMLNIPS | 15 | ORFL205C_(UL83/pp65) | 65 kDa lower matrix phosphoprotein |
| 14 | LPLKMLNIPSINVHH | 15 | ORFL205C_(UL83/pp65) | 65 kDa lower matrix phosphoprotein |
| 15 | KDVALRHVVCAHELV | 15 | ORFL205C_(UL83/pp65) | 65 kDa lower matrix phosphoprotein |
| 16 | RHVVCAHELVCSMEN | 15 | ORFL205C_(UL83/pp65) | 65 kDa lower matrix phosphoprotein |
| 17 | CSMENTRATKMQVIG | 15 | ORFL205C_(UL83/pp65) | 65 kDa lower matrix phosphoprotein |
| 18 | TRATKMQVIGDQYVK | 15 | ORFL205C_(UL83/pp65) | 65 kDa lower matrix phosphoprotein |
| 19 | MQVIGDQYVKVYLES | 15 | ORFL205C_(UL83/pp65) | 65 kDa lower matrix phosphoprotein |
| 20 | VYLESFCEDVPSGKL | 15 | ORFL205C_(UL83/pp65) | 65 kDa lower matrix phosphoprotein |
| 21 | FCEDVPSGKLFMHVT | 15 | ORFL205C_(UL83/pp65) | 65 kDa lower matrix phosphoprotein |
| 22 | LGSDVEEDLTMTRNP | 15 | ORFL205C_(UL83/pp65) | 65 kDa lower matrix phosphoprotein |
| 23 | EEDLTMTRNPQPFMR | 15 | ORFL205C_(UL83/pp65) | 65 kDa lower matrix phosphoprotein |
| 24 | QPFMRPHERNGFTVL | 15 | ORFL205C_(UL83/pp65) | 65 kDa lower matrix phosphoprotein |
| 25 | KISHIMLDVAFTSHE | 15 | ORFL205C_(UL83/pp65) | 65 kDa lower matrix phosphoprotein |
| 26 | MLDVAFTSHEHFGLL | 15 | ORFL205C_(UL83/pp65) | 65 kDa lower matrix phosphoprotein |
| 27 | FTSHEHFGLLCPKSI | 15 | ORFL205C_(UL83/pp65) | 65 kDa lower matrix phosphoprotein |
| 28 | PQYSEHPTFTSQYRI | 15 | ORFL205C_(UL83/pp65) | 65 kDa lower matrix phosphoprotein |
| 29 | SQYRIQGKLEYRHTW | 15 | ORFL205C_(UL83/pp65) | 65 kDa lower matrix phosphoprotein |
| 30 | YRHTWDRHDEGAAQG | 15 | ORFL205C_(UL83/pp65) | 65 kDa lower matrix phosphoprotein |
| 31 | IHNPAVFTWPPWQAG | 15 | ORFL205C_(UL83/pp65) | 65 kDa lower matrix phosphoprotein |
| 32 | PWQAGILARNLVPMV | 15 | ORFL205C_(UL83/pp65) | 65 kDa lower matrix phosphoprotein |
| 33 | ATVQGQNLKYQEFFW | 15 | ORFL205C_(UL83/pp65) | 65 kDa lower matrix phosphoprotein |
| 34 | QNLKYQEFFWDANDI | 15 | ORFL205C_(UL83/pp65) | 65 kDa lower matrix phosphoprotein |
| 35 | QEFFWDANDIYRIFA | 15 | ORFL205C_(UL83/pp65) | 65 kDa lower matrix phosphoprotein |
| 36 | ELEGVWQPAAQPKRR | 15 | ORFL205C_(UL83/pp65) | 65 kDa lower matrix phosphoprotein |
| 37 | IFLEVQAIRETVELR | 15 | ORFL205C_(UL83/pp65) | 65 kDa lower matrix phosphoprotein |
| 38 | PPWQAGILARNLVPM | 15 | ORFL205C_(UL83/pp65) | 65 kDa lower matrix phosphoprotein |
| 39 | DVPSGKLFMHVTLGS | 15 | ORFL205C_(UL83/pp65) | 65 kDa lower matrix phosphoprotein |
| 40 | KLFMHVTLGSDVEED | 15 | ORFL205C_(UL83/pp65) | 65 kDa lower matrix phosphoprotein |
| 41 | DVEEDLTMTRNPQPF | 15 | ORFL205C_(UL83/pp65) | 65 kDa lower matrix phosphoprotein |
| 42 | VAFTSHEHFGLLCPK | 15 | ORFL205C_(UL83/pp65) | 65 kDa lower matrix phosphoprotein |
| 43 | SEHPTFTSQYRIQGK | 15 | ORFL205C_(UL83/pp65) | 65 kDa lower matrix phosphoprotein |
| 44 | LEYRHTWDRHDEGAA | 15 | ORFL205C_(UL83/pp65) | 65 kDa lower matrix phosphoprotein |
| 45 | PLKMLNIPSINVHHY | 15 | ORFL205C_(UL83/pp65) | 65 kDa lower matrix phosphoprotein |
| 46 | KVYLESFCEDVPSGK | 15 | ORFL205C_(UL83/pp65) | 65 kDa lower matrix phosphoprotein |
| 47 | TLGSDVEEDLTMTRN | 15 | ORFL205C_(UL83/pp65) | 65 kDa lower matrix phosphoprotein |
| 48 | ASTSAGRKRKSASSA | 15 | ORFL205C_(UL83/pp65) | 65 kDa lower matrix phosphoprotein |
| 49 | ACTSGVMTRGRLKAE | 15 | ORFL205C_(UL83/pp65) | 65 kDa lower matrix phosphoprotein |
| 50 | AGILARNLVPMVATV | 15 | ORFL205C_(UL83/pp65) | 65 kDa lower matrix phosphoprotein |
| 51 | EPDVYYTSAFVFPTK | 15 | ORFL205C_(UL83/pp65) | 65 kDa lower matrix phosphoprotein |
| 52 | QVIGDQYVKVYLESF | 15 | ORFL205C_(UL83/pp65) | 65 kDa lower matrix phosphoprotein |
| 53 | FFWDANDIYRIFAEL | 15 | ORFL205C_(UL83/pp65) | 65 kDa lower matrix phosphoprotein |
| 54 | LVSQYTPDSTPCHRG | 15 | ORFL205C_(UL83/pp65) | 65 kDa lower matrix phosphoprotein |
| 55 | SHIMLDVAFTSHEH | 14 | ORFL205C_(UL83/pp65) | 65 kDa lower matrix phosphoprotein |
| 56 | DEDSDNEIHNPAVFTW | 16 | ORFL205C_(UL83/pp65) | 65 kDa lower matrix phosphoprotein |
| 57 | SQYTPDSTPCHRG | 13 | ORFL205C_(UL83/pp65) | 65 kDa lower matrix phosphoprotein |
| 58 | KPGKISHIMLDVA | 13 | ORFL205C_(UL83/pp65) | 65 kDa lower matrix phosphoprotein |
| 59 | PTFTSQYRIQGKL | 13 | ORFL205C_(UL83/pp65) | 65 kDa lower matrix phosphoprotein |
| 60 | DTPVLPHETRLLQTGIHVRV | 20 | ORFL205C_(UL83/pp65) | 65 kDa lower matrix phosphoprotein |
| 61 | INVHHYPSAAERKHRHLPVA | 20 | ORFL205C_(UL83/pp65) | 65 kDa lower matrix phosphoprotein |
| 62 | LLQRGPQYSEHPTFT | 15 | ORFL205C_(UL83/pp65) | 65 kDa lower matrix phosphoprotein |
| 63 | ALFFFDIDLLLQRGPQYSE | 19 | ORFL205C_(UL83/pp65) | 65 kDa lower matrix phosphoprotein |
| 64 | DQYVKVYLESFCEDVPSGKL | 20 | ORFL205C_(UL83/pp65) | 65 kDa lower matrix phosphoprotein |
| 65 | MTRNPQPFMRPHERNGFTVL | 20 | ORFL205C_(UL83/pp65) | 65 kDa lower matrix phosphoprotein |
| 66 | MISVLGPISGHVLKAVFSRG | 20 | ORFL205C_(UL83/pp65) | 65 kDa lower matrix phosphoprotein |
| 67 | ASGKQMWQARLTVSGLAWTR | 20 | ORFL205C_(UL83/pp65) | 65 kDa lower matrix phosphoprotein |
| 68 | LPLKMLNIPSINVHHYPSAA | 20 | ORFL205C_(UL83/pp65) | 65 kDa lower matrix phosphoprotein |
| 69 | PHETRLLQTGIHVRVSQPSL | 20 | ORFL205C_(UL83/pp65) | 65 kDa lower matrix phosphoprotein |
| 70 | IYVYALPLKMLNIPSINVHH | 20 | ORFL205C_(UL83/pp65) | 65 kDa lower matrix phosphoprotein |
| 71 | QYDPVAALFFFDIDLLLQRG | 20 | ORFL205C_(UL83/pp65) | 65 kDa lower matrix phosphoprotein |
| 72 | RQYDPVAALFFFDIDL | 16 | ORFL205C_(UL83/pp65) | 65 kDa lower matrix phosphoprotein |
| 73 | HETRLLQTGIHVRVS | 15 | ORFL205C_(UL83/pp65) | 65 kDa lower matrix phosphoprotein |
| 74 | VYALPLKMLNIPSIN | 15 | ORFL205C_(UL83/pp65) | 65 kDa lower matrix phosphoprotein |
| 75 | VALRHVVCAHELVCS | 15 | ORFL205C_(UL83/pp65) | 65 kDa lower matrix phosphoprotein |
| 76 | HIMLDVAFTSHEHFG | 15 | ORFL205C_(UL83/pp65) | 65 kDa lower matrix phosphoprotein |
| 77 | FTSQYRIQGKLEYRH | 15 | ORFL205C_(UL83/pp65) | 65 kDa lower matrix phosphoprotein |
| 78 | YRIQGKLEYRHTWDR | 15 | ORFL205C_(UL83/pp65) | 65 kDa lower matrix phosphoprotein |
| 79 | ARNLVPMVATVQGQN | 15 | ORFL205C_(UL83/pp65) | 65 kDa lower matrix phosphoprotein |
| 80 | ANDIYRIFAELEGVW | 15 | ORFL205C_(UL83/pp65) | 65 kDa lower matrix phosphoprotein |
| 81 | TRQQNQWKEPDVYYT | 15 | ORFL205C_(UL83/pp65) | 65 kDa lower matrix phosphoprotein |
| 82 | TERKTPRVTGGGAMA | 15 | ORFL205C_(UL83/pp65) | 65 kDa lower matrix phosphoprotein |
| 83 | NLKYQEFFWDANDIY | 15 | ORFL205C_(UL83/pp65) | 65 kDa lower matrix phosphoprotein |
| 84 | TPRVTGGGAMAGAST | 15 | ORFL205C_(UL83/pp65) | 65 kDa lower matrix phosphoprotein |
| 85 | DQYVKVYLESFCEDV | 15 | ORFL205C_(UL83/pp65) | HCMVUL83 |
| 86 | GKISHIMLDVAFTSH | 15 | ORFL205C_(UL83/pp65) | HCMVUL83 |
| 87 | EHPTFTSQYRIQGKL | 15 | ORFL205C_(UL83/pp65) | HCMVUL83 |
| 88 | GQNLKYQEFFWDAND | 15 | ORFL205C_(UL83/pp65) | HCMVUL83 |
| 89 | KYQEFFWDANDIYRI | 15 | ORFL205C_(UL83/pp65) | HCMVUL83 |
| 90 | IIKPGKISHIMLDVA | 15 | ORFL205C_(UL83/pp65) | HCMVUL83 |
| 91 | TRATKMQVIGDQYVKVYLES | 20 | ORFL205C_(UL83/pp65) | HCMVUL83 |
| 92 | KLFMHVTLGSDVEEDLTMTR | 20 | ORFL205C_(UL83/pp65) | HCMVUL83 |
| 93 | KPGKISHIMLDVAFTSHEHF | 20 | ORFL205C_(UL83/pp65) | HCMVUL83 |
| 94 | LPVADAVIHASGKQMWQARL | 20 | ORFL205C_(UL83/pp65) | HCMVUL83 |
| 95 | GSDSDEELVTTERKTPRVTG | 20 | ORFL205C_(UL83/pp65) | HCMVUL83 |
| 96 | RHRQDALPGPCIASTPKKHR | 20 | ORFL205C_(UL83/pp65) | HCMVUL83 |
| 97 | YQEFFWDANDIYR | 13 | ORFL205C_(UL83/pp65) | HCMVUL83 |
| 98 | LAWTRQQNQWKEPDV | 15 | ORFL205C_(UL83/pp65) | HCMVUL83 |
| 99 | YQEFFWDANDIYRIF | 15 | ORFL205C_(UL83/pp65) | HCMVUL83 |
| 100 | EFFWDANDIYRIF | 13 | ORFL205C_(UL83/pp65) | HCMVUL83 |
| 101 | VEEDLTMTRNPQPFM | 15 | ORFL205C_(UL83/pp65) | HCMVUL83 |
| 102 | KPGKISHIMLDVAFTSH | 17 | ORFL205C_(UL83/pp65) | HCMVUL83 |
| 103 | TSQYRIQGKLEYRHT | 15 | ORFL205C_(UL83/pp65) | HCMVUL83 |
| 104 | MSIYVYALPLKMLNI | 15 | ORFL205C_(UL83/pp65) | HCMVUL83 |
| 105 | VYYTSAFVFPTKDVA | 15 | ORFL205C_(UL83/pp65) | HCMVUL83 |
| 106 | LRQYDPVAALFFFDI | 15 | ORFL205C_(UL83/pp65) | HCMVUL83 |
| 107 | GPQYSEHPTFTSQYRI | 16 | ORFL205C_(UL83/pp65) | HCMVUL83 |
| 108 | HPTFTSQYRIQGKLE | 15 | ORFL205C_(UL83/pp65) | HCMVUL83 |
| 109 | TRLLQTGIHVRVSQP | 15 | ORFL205C_(UL83/pp65) | HCMVUL83 |
| 110 | RNGFTVLCPKNMIIK | 15 | ORFL205C_(UL83/pp65) | HCMVUL83 |
| 111 | PISGHVLKAVFSRGD | 15 | ORFL205C_(UL83/pp65) | HCMVUL83 |
| 112 | GIHVRVSQPSLILVS | 15 | ORFL205C_(UL83/pp65) | HCMVUL83 |
| 113 | IHASGKQMWQARLTV | 15 | ORFL205C_(UL83/pp65) | HCMVUL83 |
| 114 | GKQMWQARLTVSGLA | 15 | ORFL205C_(UL83/pp65) | HCMVUL83 |
| 115 | ENTRATKMQVIGDQY | 15 | ORFL205C_(UL83/pp65) | HCMVUL83 |
| 116 | ATKMQVIGDQYVKVY | 15 | ORFL205C_(UL83/pp65) | HCMVUL83 |
| 117 | RPHERNGFTVLCPKN | 15 | ORFL205C_(UL83/pp65) | HCMVUL83 |
| 118 | AQGDDDVWTSGSDSD | 15 | ORFL205C_(UL83/pp65) | HCMVUL83 |
| 119 | SSATACTSGVMTRGR | 15 | ORFL205C_(UL83/pp65) | HCMVUL83 |
| 120 | YRIFAELEGVWQPAA | 15 | ORFL205C_(UL83/pp65) | HCMVUL83 |
| 121 | AELEGVWQPAAQPKR | 15 | ORFL205C_(UL83/pp65) | HCMVUL83 |
| 122 | AVFSRGDTPVLPHET | 15 | ORFL205C_(UL83/pp65) | phosphorylated matrix protein |
| | | | | (pp65) |
| 123 | ALPLKMLNIPSINVH | 15 | ORFL205C_(UL83/pp65) | pp65 |
| 124 | HVLKAVFSRGDTPVL | 15 | ORFL205C_(UL83/pp65) | pp65 |
| 125 | AHELVCSMENTRATKMQVIG | 20 | ORFL205C_(UL83/pp65) | tegument protein pp65 |
| 126 | FCEDVPSGKLFMHVTLGSDV | 20 | ORFL205C_(UL83/pp65) | tegument protein pp65 |
| 127 | TLGSDVEEDLTMTRNPQPF | 19 | ORFL205C_(UL83/pp65) | tegument protein pp65 |
| 128 | LLQTGIHVRVSQPSLILV | 18 | ORFL205C_(UL83/pp65) | tegument protein pp65 |
| 129 | SICPSQEPMSIYVYA | 15 | ORFL205C_(UL83/pp65) | tegument protein pp65 |
| 130 | SQEPMSIYVYALPLK | 15 | ORFL205C_(UL83/pp65) | tegument protein pp65 |
| 131 | LNIPSINVHHYPSAA | 15 | ORFL205C_(UL83/pp65) | tegument protein pp65 |
| 132 | HDVSKGDDNKLGGALQAKA | 19 | ORFL264C_(UL123) IE1 | 55 kDa immediate-early protein 1 |
| 133 | ALQAKAR DKKDELRRKMMY | 19 | ORFL264C_(UL123) IE1 | 55 kDa immediate-early protein 1 |
| 134 | KEHMLKKYTQTEEKF | 15 | ORFL264C_(UL123) IE1 | 55 kDa immediate-early protein 1 |
| 135 | QTEEKFTGAFNMMGGCLQN | 19 | ORFL264C_(UL123) IE1 | 55 kDa immediate-early protein 1 |
| 136 | MGGCLQNALDILDKVHEPFE | 20 | ORFL264C_(UL123) IE1 | 55 kDa immediate-early protein 1 |
| 137 | AIVAYTLATAGVSSSDSLV | 19 | ORFL264C_(UL123) IE1 | 55 kDa immediate-early protein 1 |
| 138 | TMQSMYENYIVPEDKREMW | 19 | ORFL264C_(UL123) IE1 | 55 kDa immediate-early protein 1 |
| 139 | RRKMMYMCYRNIEFFTKNS | 19 | ORFL264C_(UL123) IE1 | 55 kDa immediate-early protein 1 |
| 140 | FFTKNSAFPKTTNGCSQAM | 19 | ORFL264C_(UL123) IE1 | 55 kDa immediate-early protein 1 |
| 141 | CVETMCNEYKVTSDACMMT | 19 | ORFL264C_(UL123) IE1 | 55 kDa immediate-early protein 1 |
| 142 | DACMMTMYGGASLLSEFCR | 19 | ORFL264C_(UL123) IE1 | 55 kDa immediate-early protein 1 |
| 143 | NYIVPEDKREMWMACIKELH | 20 | ORFL264C_(UL123) IE1 | 55 kDa immediate-early protein 1 |
| 144 | VRHRIKEHMLKKYTQTEEKF | 20 | ORFL264C_(UL123) IE1 | 55 kDa immediate-early protein 1 |
| 145 | VRVDMVRHRIKEHML | 15 | ORFL264C_(UL123) IE1 | 55 kDa immediate-early protein 1 |
| 146 | VKQIKVRVDMVRHRI | 15 | ORFL264C_(UL123) IE1 | 55 kDa immediate-early protein 1 |
| 147 | VRHRIKEHMLKKYTQ | 15 | ORFL264C_(UL123) IE1 | 55 kDa immediate-early protein 1 |
| 148 | EQSDEEEEEGAQEER | 15 | ORFL264C_(UL123) IE1 | 55 kDa immediate-early protein 1 |
| 149 | VKSEPVSEIEEVAPE | 15 | ORFL264C_(UL123) IE1 | 55 kDa immediate-early protein 1 |
| 150 | PVSEIEEVAPEEEED | 15 | ORFL264C_(UL123) IE1 | 55 kDa immediate-early protein 1 |
| 151 | LQNALDILDKVHEPF | 15 | ORFL264C_(UL123) IE1 | 55 kDa immediate-early protein 1 |
| 152 | EDKREMWMACIKELH | 15 | ORFL264C_(UL123) IE1 | 55 kDa immediate-early protein 1 |
| 153 | THIDHIFMDILTTCV | 15 | ORFL264C_(UL123) IE1 | 55 kDa immediate-early protein 1 |
| 154 | VLEETSVMLAKRPLI | 15 | ORFL264C_(UL123) IE1 | 55 kDa immediate-early protein 1 |
| 155 | TKPEVISVMKRRIEE | 15 | ORFL264C_(UL123) IE1 | 55 kDa immediate-early protein 1 |
| 156 | RRIEEICMKVFAQYI | 15 | ORFL264C_(UL123) IE1 | 55 kDa immediate-early protein 1 |
| 157 | NIEFFTKNSAFPKTT | 15 | ORFL264C_(UL123) IE1 | regulatory protein IE1 |
| 158 | LTHIDHIFMDILTTCVETM | 19 | ORFL264C_(UL123) IE1 | regulatory protein IE1 |
| 159 | AIVAYTLATAGASSSDSLV | 19 | ORFL264C_(UL123) IE1 | UL123; IE1 |
| 160 | VRVDMVRHRIKEHMLKKYTQ | 20 | ORFL264C_(UL123) IE1 | UL123; IE1 |
| 161 | DKREMWMACIKELH | 14 | ORFL264C_(UL123) IE1 | UL123; IE1 |
| 162 | QSMYENYIVPEDKREMWMAC | 20 | ORFL264C_(UL123) IE1 | UL123; IE1 |
| 163 | TRRGRVKIDEVSRMF | 15 | ORFL265C_(UL122) IE2 | 45 kDa immediate-early protein 2 |
| 164 | GDILAQAVNHAGIDS | 15 | ORFL265C_(UL122) IE2 | 45 kDa immediate-early protein 2 |
| 165 | KTTRPFKVIIKPPVP | 15 | ORFL265C_(UL122) IE2 | 45 kDa immediate-early protein 2 |
| 166 | FKVIIKPPVPPAPIM | 15 | ORFL265C_(UL122) IE2 | 45 kDa immediate-early protein 2 |
| 167 | PEPDFTIQYRNKIID | 15 | ORFL265C_(UL122) IE2 | 45 kDa immediate-early protein 2 |
| 168 | PFTIPSMHQVLDEAI | 15 | ORFL265C_(UL122) IE2 | 45 kDa immediate-early protein 2 |
| 169 | LMQKFPKQVMVRIFS | 15 | ORFL265C_(UL122) IE2 | 45 kDa immediate-early protein 2 |
| 170 | VRIFSTNQGGFMLPI | 15 | ORFL265C_(UL122) IE2 | 45 kDa immediate-early protein 2 |
| 171 | PEDLDTLSLAIEAAI | 15 | ORFL265C_(UL122) IE2 | 45 kDa immediate-early protein 2 |
| 172 | TLSLAIEAAIQDLRN | 15 | ORFL265C_(UL122) IE2 | 45 kDa immediate-early protein 2 |
| 173 | SMHQVLDEAIKACKT | 15 | ORFL265C_(UL122) IE2 | 45 kDa immediate-early protein 2 |
| 174 | KGIQIIYTRNHEVKS | 15 | ORFL265C_(UL122) IE2 | 45 kDa immediate-early protein 2 |
| 175 | ALSTPFLMEHTMPVT | 15 | ORFL265C_(UL122) IE2 | 45 kDa immediate-early protein 2 |
| 176 | FLMEHTMPVTHPPEV | 15 | ORFL265C_(UL122) IE2 | 45 kDa immediate-early protein 2 |
| 177 | PYAVAFQPLLAYAY | 14 | UL57 | single-stranded DNA-binding protein |
| 178 | KTQLNRHSYLKDSDFLDAA | 19 | UL75 | envelope glycoprotein H |
| 179 | RQTEKHELLVLVKKAQLNRH | 20 | UL75 | Glycoprotein H precursor |
| 180 | LDPHAFHLLLNTYGRPIR | 18 | UL75 | Glycoprotein H precursor |
| 181 | KAQLNRHSYLKDSDFLDAA | 19 | UL75 | Glycoprotein H precursor |
| 182 | DVLKSGRCQMLDRRTVEMA | 19 | UL75 | Glycoprotein H precursor |
| 183 | LDKAFHLLLNTYGRPIR | 17 | UL75 | Glycoprotein H precursor |
| 184 | KDQLNRHSYLKDPDFLDAA | 19 | UL75 | Glycoprotein H precursor |
| 185 | SYLKDSDFLDAAL | 13 | UL75 | HCMVUL75 |
| 186 | RRIPHFYRVRREVPRTVNE | 19 | UL86 | Major capsid protein |
| 187 | MDVNYFKIPNNPRGRASCM | 19 | UL86 | Major capsid protein |

### REFERENCES

1. Stem-Ginossar N, Weisburd B, Michalski A, Le V T, Hein M Y, Huang S X, et al. Decoding human cytomegalovirus. Science. 2012; 338(6110):1088-93.
2. Collins-McMillen D, Buehler J, Peppenelli M, and Goodrum F. Molecular Determinants and the Regulation of Human Cytomegalovirus Latency and Reactivation. Viruses. 2018; 10(8).
3. Hargett D, and Shenk T E. Experimental human cytomegalovirus latency in CD14+ monocytes. Proc Natl Acad Sci USA. 2010; 107(46):20039-44.
4. Chaturvedi S, Klein J, Vardi N, Bolovan-Fritts C, Wolf M, Du K, et al. A molecular mechanism for probabilistic bet hedging and its role in viral latency. Proc Natl Acad Sci USA. 2020; 117(29):17240-8.
5. Davison A J, Dolan A, Akter P, Addison C, Dargan D J, Alcendor D J, et al. The human cytomegalovirus genome revisited: comparison with the chimpanzee cytomegalovirus genome. J Gen Virol. 2003; 84(Pt 1):17-28.
6. Mattes F M, Vargas A, Kopycinski J, Hainsworth E G, Sweny P, Nebbia G, et al. Functional impairment of cytomegalovirus specific CD8 T cells predicts high-level replication after renal transplantation. Am J Transplant. 2008; 8(5):990-9.
7. Walker S, Fazou C, Crough T, Holdsworth R, Kiely P, Veale M, et al. Ex vivo monitoring of human cytomegalovirus-specific CD8+ T-cell responses using QuantiFERON-CMV. Transpl Infect Dis. 2007; 9(2):165-70.
8. Clarke L M, Duerr A, Feldman J, Sierra M F, Daidone B J, and Landesman S H. Factors associated with cytomegalovirus infection among human immunodeficiency virus type 1-seronegative and -seropositive women from an urban minority community. J Infect Dis. 1996; 173(1):77-82.
9. Doyle M, Atkins J T, and Rivera-Matos I R. Congenital cytomegalovirus infection in infants infected with human immunodeficiency virus type 1. Pediatr Infect Dis J. 1996; 15(12):1102-6.
10. Duryea E L, Sanchez P J, Sheffield J S, Jackson G L, Wendel G D, McElwee B S, et al. Maternal human immunodeficiency virus infection and congenital transmission of cytomegalovirus. Pediatr Infect Dis J 2010; 29(10):915-8.
11. Kovacs A, Schluchter M, Easley K, Demmler G, Shearer W, La Russa P, et al. Cytomegalovirus infection and HIV-1 disease progression in infants born to HIV-1-infected women. Pediatric Pulmonary and Cardiovascular Complications of Vertically Transmitted HIV Infection Study Group. N Engl J Med. 1999; 341(2):77-84.
12. Schoenfisch A L, Dollard S C, Amin M, Gardner L I, Klein R S, Mayer K, et al. Cytomegalovirus (CMV) shedding is highly correlated with markers of immunosuppression in CMV-seropositive women. J Med Microbiol. 2011; 60(Pt 6):768-74.
13. Demmler-Harrison G J. Congenital cytomegalovirus: Public health action towards awareness, prevention, and treatment. J Clin Virol. 2009; 46 Suppl 4:S1-5.
14. Jeon J, Victor M, Adler S P, Arwady A, Demmler G, Fowler K, et al. Knowledge and awareness of congenital cytomegalovirus among women. Infect Dis Obstet Gynecol. 2006; 2006:80383.
15. Fowler K B, Stagno S, Pass R F, Britt W J, Boll T J, and Alford C A. The outcome of congenital cytomegalovirus infection in relation to maternal antibody status. N Engl J Med. 1992; 326(10):663-7.
16. Ross S A, Fowler K B, Ashrith G, Stagno S, Britt W J, Pass R F, et al. Hearing loss in children with congenital cytomegalovirus infection born to mothers with preexisting immunity. J Pediatr. 2006; 148(3):332-6.
17. Fowler K B, and Boppana S B. Congenital cytomegalovirus (CMV) infection and hearing deficit. J Clin Virol. 2006; 35(2):226-31.
18. Ross S A, and Boppana S B. Congenital cytomegalovirus infection: outcome and diagnosis. Semin Pediatr Infect Dis. 2005; 16(1):44-9.
19. Britt W J. Maternal Immunity and the Natural History of Congenital Human Cytomegalovirus Infection. Viruses. 2018; 10(8).
20. Griffiths P D. Strategies to prevent CMV infection in the neonate. Semin Neonatol. 2002; 7(4):293-9.
21. Kimberlin D W, Lin C Y, Sanchez P J, Demmler G J, Dankner W, Shelton M, et al. Effect of ganciclovir therapy on hearing in symptomatic congenital cytomegalovirus disease involving the central nervous system: a randomized, controlled trial. J Pediatr. 2003; 143(1):16-25.
22. Michaels M G, Greenberg D P, Sabo D L, and Wald E R. Treatment of children with congenital cytomegalovirus infection with ganciclovir. Pediatr Infect Dis J 2003; 22(6):504-9.
23. Whitley R J, Cloud G, Gruber W, Storch G A, Demmler G J, Jacobs R F, et al. Ganciclovir treatment of symptomatic congenital cytomegalovirus infection: results of a phase II study. National Institute of Allergy and Infectious Diseases Collaborative Antiviral Study Group. J Infect Dis. 1997; 175(5):1080-6.
24. Benedict C A. A CMV vaccine: TREATing despite the TRICKs. Expert Rev Vaccines. 2013; 12(11):1235-7.
25. Permar S R, Schleiss M R, and Plotkin S A. Advancing Our Understanding of Protective Maternal Immunity as a Guide for Development of Vaccines To Reduce Congenital Cytomegalovirus Infections. J Virol. 2018; 92(7).
26. Plotkin S A, and Boppana S B. Vaccination against the human cytomegalovirus. Vaccine. 2019; 37(50):7437-42.
27. Li C R, Greenberg P D, Gilbert M J, Goodrich J M, and Riddell S R. Recovery of HLA-restricted cytomegalovirus (CMV)-specific T-cell responses after allogeneic bone marrow transplant: correlation with CMV disease and effect of ganciclovir prophylaxis. Blood. 1994; 83(7):1971-9.
28. Quinnan G V, Jr., Kirmani N, Rook A H, Manischewitz J F, Jackson L, Moreschi G, et al. Cytotoxic t cells in cytomegalovirus infection: HLA-restricted T-lymphocyte and non-T-lymphocyte cytotoxic responses correlate with recovery from cytomegalovirus infection in bone-marrow-transplant recipients. N Engl J Med. 1982; 307(1):7-13.
29. Reusser P, Riddell S R, Meyers J D, and Greenberg P D. Cytotoxic T-lymphocyte response to cytomegalovirus after human allogeneic bone marrow transplantation: pattern of recovery and correlation with cytomegalovirus infection and disease. Blood. 1991; 78(5):1373-80.
30. Smith C J, Quinn M, and Snyder C M. CMV-Specific CD8 T Cell Differentiation and Localization: Implications for Adoptive Therapies. Front Immunol. 2016; 7:352.
31. Sylwester A W, Mitchell B L, Edgar J B, Taormina C, Pelte C, Ruchti F, et al. Broadly targeted human cytomegalovirus-specific CD4+ and CD8+ T cells dominate the memory compartments of exposed subjects. J Exp Med. 2005; 202(5):673-85.
32. Kern F, Bunde T, Faulhaber N, Kiecker F, Khatamzas E, Rudawski I M, et al. Cytomegalovirus (CMV) phosphoprotein 65 makes a large contribution to shaping the T cell repertoire in CMV-exposed individuals. J Infect Dis. 2002; 185(12):1709-16.
33. Kern F, Surel I P, Faulhaber N, Frommel C, Schneider-Mergener J, Schonemann C, et al. Target structures of the CD8(+)-T-cell response to human cytomegalovirus: the 72-kilodalton major immediate-early protein revisited. J Virol. 1999; 73(10):8179-84.
34. Khan N, Best D, Bruton R, Nayak L, Rickinson A B, and Moss P A. T cell recognition patterns of immunodominant cytomegalovirus antigens in primary and persistent infection. J Immunol. 2007; 178(7):4455-65.
35. Khan N, Cobbold M, Keenan R, and Moss P A. Comparative analysis of CD8+ T cell responses against human cytomegalovirus proteins pp65 and immediate early 1 shows similarities in precursor frequency, oligoclonality, and phenotype. J Infect Dis. 2002; 185(8):1025-34.
36. Elkington R, Walker S, Crough T, Menzies M, Tellam J, Bharadwaj M, et al. Ex vivo profiling of CD8+-T-cell responses to human cytomegalovirus reveals broad and multispecific reactivities in healthy virus carriers. J Virol. 2003; 77(9):5226-40.
37. Elkington R, Shoukry N H, Walker S, Crough T, Fazou C, Kaur A, et al. Cross-reactive recognition of human and primate cytomegalovirus sequences by human CD4 cytotoxic T lymphocytes specific for glycoprotein B and H. Eur J Immunol. 2004; 34(11):3216-26.
38. Paston S J, Dodi I A, and Madrigal J A. Progress made towards the development of a CMV peptide vaccine. Hum Immunol. 2004; 65(5):544-9.
39. Fuhrmann S, Streitz M, Reinke P, Volk H D, and Kern F. T cell response to the cytomegalovirus major capsid protein (UL86) is dominated by helper cells with a large polyfunctional component and diverse epitope recognition. J Infect Dis. 2008; 197(10):1455-8.
40. Paul S, Lindestam Arlehamn C S, Scriba T J, Dillon M B, Oseroff C, Hinz D, et al. Development and validation of a broad scheme for prediction of HLA class II restricted T cell epitopes. J Immunol Methods. 2015; 422:28-34.
41. Vita R, Mahajan S, Overton J A, Dhanda S K, Martini S, Cantrell J R, et al. The Immune Epitope Database (IEDB): 2018 update. Nucleic Acids Res. 2019; 47(D1):D339-D43.
42. Bancroft T, Dillon M B, da Silva Antunes R, Paul S, Peters B, Crotty S, et al. Th1 versus Th2 T cell polarization by whole-cell and acellular childhood pertussis vaccines persists upon re-immunization in adolescence and adulthood. Cell Immunol. 2016; 304-305:35-43.
43. da Silva Antunes R, Paul S, Sidney J, Weiskopf D, Dan J M, Phillips E, et al. Definition of Human Epitopes Recognized in Tetanus Toxoid and Development of an Assay Strategy to Detect Ex Vivo Tetanus CD4+ T Cell Responses. PLoS One. 2017; 12(1):e0169086.
44. Grifoni A, Weiskopf D, Ramirez S I, Mateus J, Dan J M, Moderbacher C R, et al. Targets of T Cell Responses to SARS-CoV-2 Coronavirus in Humans with COVID-19 Disease and Unexposed Individuals. Cell. 2020; 181(7):1489-501 e15.
45. Lindestam Arlehamn C S, McKinney D M, Carpenter C, Paul S, Rozot V, Makgotlho E, et al. A Quantitative Analysis of Complexity of Human Pathogen-Specific CD4 T Cell Responses in Healthy M. tuberculosis Infected South Africans. PLoS Pathog. 2016; 12(7):e1005760.
46. Mateus J, Grifoni A, Tarke A, Sidney J, Ramirez S I, Dan J M, et al. Selective and cross-reactive SARS-CoV-2 T cell epitopes in unexposed humans. Science. 2020; 370(6512):89-94.
47. Carrasco Pro S, Sidney J, Paul S, Lindestam Arlehamn C, Weiskopf D, Peters B, et al. Automatic Generation of Validated Specific Epitope Sets. J Immunol Res. 2015; 2015:763461.
48. Dan J M, Mateus J, Kato Y, Hastie K M, Yu E D, Faliti C E, et al. Immunological memory to SARS-CoV-2 assessed for up to 8 months after infection. Science. 2021; 371(6529).
49. Nielsen M, Justesen S, Lund O, Lundegaard C, and Buus S. NetMHCIIpan-2.0—Improved pan-specific HLA-DR predictions using a novel concurrent alignment and weight optimization training procedure. Immunome Res. 2010; 6:9.
50. Nielsen M, and Lund O. NN-align. An artificial neural network-based alignment algorithm for MHC class II peptide binding prediction. BMC Bioinformatics. 2009; 10:296.
51. Paul S, Kolla R V, Sidney J, Weiskopf D, Fleri W, Kim Y, et al. Evaluating the immunogenicity of protein drugs by applying in vitro MHC binding data and the immune epitope database and analysis resource. Clin Dev Immunol. 2013; 2013:467852.
52. Salimi N, Fleri W, Peters B, and Sette A. The immune epitope database: a historical retrospective of the first decade. Immunology. 2012; 137(2):117-23.
53. Wang P, Sidney J, Kim Y, Sette A, Lund O, Nielsen M, et al. Peptide binding predictions for HLA DR, DP and DQ molecules. BMC Bioinformatics. 2010; 11:568.
54. Dhanda S K, Karosiene E, Edwards L, Grifoni A, Paul S, Andreatta M, et al. Predicting HLA CD4 Immunogenicity in Human Populations. Front Immunol. 2018; 9:1369.
55. Lim E Y, Jackson S E, and Wills M R. The CD4+ T Cell Response to Human Cytomegalovirus in Healthy and Immunocompromised People. Front Cell Infect Microbiol. 2020; 10:202.
56. Tian Y, Seumois G, De-Oliveira-Pinto L M, Mateus J, Herrera-de la Mata S, Kim C, et al. Molecular Signatures of Dengue Virus-Specific IL-10/IFN-gamma Co-producing CD4 T Cells and Their Association with Dengue Disease. Cell Rep. 2019; 29(13):4482-95 e4.
57. Wehrens E J, Wong K A, Gupta A, Khan A, Benedict C A, and Zuniga E L. IL-27 regulates the number, function and cytotoxic program of antiviral CD4 T cells and promotes cytomegalovirus persistence. PLoS One. 2018; 13(7):e0201249.
58. Humphreys I R, de Trez C, Kinkade A, Benedict C A, Croft M, and Ware C F. Cytomegalovirus exploits IL-10-mediated immune regulation in the salivary glands. J Exp Med. 2007; 204(5):1217-25.
59. Jackson S E, Sedikides G X, Mason G M, Okecha G, and Wills M R. Human Cytomegalovirus (HCMV)-Specific CD4(+) T Cells Are Polyfunctional and Can Respond to HCMV-Infected Dendritic Cells In Vitro. J Virol. 2017; 91(6).
60. Pachnio A, Ciaurriz M, Begum J, Lal N, Zuo J, Beggs A, et al. Cytomegalovirus Infection Leads to Development of High Frequencies of Cytotoxic Virus-Specific CD4+ T Cells Targeted to Vascular Endothelium. PLoS Pathog. 2016; 12(9):e1005832.
61. van Leeuwen E M, Remmerswaal E B, Vossen M T, Rowshani A T, Wertheim-van Dillen P M, van Lier R A, et al. Emergence of a CD4+CD28− granzyme B+, cytomegalovirus-specific T cell subset after recovery of primary cytomegalovirus infection. J Immunol. 2004; 173(3):1834-41.
62. Verma S, Weiskopf D, Gupta A, McDonald B, Peters B, Sette A, et al. Cytomegalovirus-Specific CD4 T Cells Are Cytolytic and Mediate Vaccine Protection. J Virol. 2016; 90(2):650-8.
63. Erhard F, Dolken L, Schilling B, and Schlosser A. Identification of the Cryptic HLA-I Immunopeptidome. Cancer Immunol Res. 2020; 8(8):1018-26.
64. Erhard F, Halenius A, Zimmermann C, L'Hernault A, Kowalewski D J, Weekes M P, et al. Improved Ribo-seq enables identification of cryptic translation events. Nat Methods. 2018; 15(5):363-6.
65. Laumont C M, and Perreault C. Exploiting non-canonical translation to identify new targets for T cell-based cancer immunotherapy. Cell Mol Life Sci. 2018; 75(4):607-21.
66. Nobre L V, Nightingale K, Ravenhill B J, Antrobus R, Soday L, Nichols J, et al. Human cytomegalovirus interactome analysis identifies degradation hubs, domain associations and viral protein functions. Elife. 2019; 8.
67. Picarda G, and Benedict C A. Cytomegalovirus: Shape-Shifting the Immune System. J Immunol. 2018; 200(12):3881-9.
68. Deere J D, Chang W L W, Villalobos A, Schmidt K A, Deshpande A, Castillo L D, et al. Neutralization of rhesus cytomegalovirus IL-10 reduces horizontal transmission and alters long-term immunity. Proc Natl Acad Sci USA. 2019; 116(26):13036-41.
69. Eberhardt M K, Deshpande A, Chang W L, Barthold S W, Walter M R, and Barry P A. Vaccination against a virus-encoded cytokine significantly restricts viral challenge. J Virol. 2013; 87(21):11323-31.
70. Nguyen C C, and Kamil J P. Pathogen at the Gates: Human Cytomegalovirus Entry and Cell Tropism. Viruses. 2018; 10(12).
71. Vanarsdall A L, and Johnson D C. Human cytomegalovirus entry into cells. Curr Opin Virol. 2012; 2(1):37-42.
72. Gema G, and Lilleri D. Human cytomegalovirus (HCMV) infection/re-infection: development of a protective HCMV vaccine. New Microbiol. 2019; 42(1):1-20.

1-157. (canceled)

158. A composition comprising:
one or more peptides or proteins consisting of an amino acid sequence selected from the group consisting of SEQ ID NOs.: 1-235 of Table 1 and SEQ ID NOs: 1-187 of Table 2; or
a fusion protein comprising one or more amino acid sequences selected from the group consisting of SEQ ID NOs.: 1-235 of Table 1 and SEQ ID NOs: 1-187 of Table 2; or
a pool of 2 or more or more peptides comprising amino acid sequences selected from the group consisting of SEQ ID NOs.: 1-235 of Table 1 and SEQ ID NOs: 1-187 of Table 2; or
a polynucleotide that encodes one or more peptides or proteins, comprising, consisting of, or consisting essentially of an amino acid sequence selected from the group consisting of SEQ ID NOs.: 1-235 of Table 1 and SEQ ID NOs: 1-187 of Table 2.

159. The composition according to claim 158, wherein the amino acid sequence is selected from a cytomegalovirus T cell epitope.

160. The composition of claim 158, the one or more peptides or proteins comprises a cytomegalovirus CD8+ or CD4+ T cell epitope.

161. The composition of claim 159, wherein the cytomegalovirus is HCMV and the HCMV T cell epitope is not conserved in another cytomegalovirus.

162. The composition of claim 159, wherein the cytomegalovirus is HCMV and the HCMV T cell epitope is conserved in another cytomegalovirus.

163. The composition of claim 158, wherein the one or more peptides or proteins is selected from the group consisting of a HCMV Glycoprotein B, a 65 kDa lower matrix phosphoprotein, a HCMVUL83, a phosphorylated matrix protein (pp65), a tegument protein pp65, a 55 kDa immediate-early protein 1, a regulatory protein IE1, UL123, IE1, a 45 kDa immediate-early protein 2, a single-stranded DNA-binding protein, an envelope glycoprotein H, a glycoprotein H precursor, a major capsid protein, a HCMV UL75 protein or peptide, and variants, homologues, derivatives, or subsequences thereof.

164. The composition of claim 158, further comprising an adjuvant.

165. The composition of claim 164, wherein the adjuvant is selected from the group consisting of alum, aluminum hydroxide, aluminum phosphate, calcium phosphate hydroxide, cytosine-guanosine oligonucleotide (CpG-ODN) sequence, granulocyte macrophage colony stimulating factor (GM-CSF), monophosphoryl lipid A (MPL), poly(I:C), MF59, Quil A, N-acetyl muramyl-L-alanyl-D-isoglutamine (MDP), FIA, montanide, poly (DL-lactide-coglycolide), squalene, virosome, ASO3, ASO4, IL-1, IL-2, IL-3, IL-4, IL-5, IL-6, IL-7, IL-8, IL-10, IL-12, IL-15, IL-17, IL-18, STING, CD40L, pathogen-associated molecular patterns (PAMPs), damage-associated molecular pattern molecules (DAMPs), Freund's complete adjuvant, Freund's incomplete adjuvant, transforming growth factor (TGF)-beta antibody or antagonists, A2aR antagonists, lipopolysaccharides (LPS), Fas ligand, Trail, lymphotactin, Mannan (M-FP), APG-2, Hsp70 and Hsp90, pattern recognition receptor ligands, TLR3 ligands, TLR4 ligands, TLR5 ligands, TLR7/8 ligands, and TLR9 ligands.

166. The composition of claim 158, further comprising a modulator of immune response.

167. A method for detecting the presence of: (i) a cytomegalovirus or (ii) an immune response relevant to cytomegalovirus infections, vaccines or therapies, including T cells responsive to one or more cytomegalovirus peptides, comprising:
providing one or more proteins or peptides for detection of an amount or a relative amount of, and/or the activity of, and/or the state of antigen-specific T-cells;
contacting a biological sample suspected of having cytomegalovirus-specific T-cells with one or more proteins or peptides for detection; and
detecting an amount or a relative amount of, and/or the activity of, and/or the state of antigen-specific T-cells in the biological sample, wherein the one or more proteins or peptides for detection comprises one or more amino acid sequences as claimed in claim 158.

168. The method of claim 167, wherein detecting comprises one or more steps of identification or detection of the antigen-specific T-cells and measuring the amount of the antigen-specific T-cells.

169. The method of claim 167, wherein detecting comprises measuring one or more selected from the group consisting of a cytokine or lymphokine secretion assay, T cell proliferation, immunoprecipitation, immunoassay, ELISA, radioimmunoassay, immunofluorescence assay, Western Blot, FACS analysis, a competitive immunoassay, a noncompetitive immunoassay, a homogeneous immunoassay a heterogeneous immunoassay, a bioassay, a reporter assay, a luciferase assay, a microarray, a surface plasmon resonance detector, a florescence resonance energy transfer, immunocytochemistry, or a cell mediated assay, and a cytokine proliferation assay.

170. A method for detecting the presence of: (i) HCMV or (ii) an immune response relevant to HCMV infections, vaccines or therapies, including T cells responsive to one or more HCMV peptides, comprising:
providing one or more proteins or peptides for detection of an amount or a relative amount of, and/or the activity of, and/or the state of antigen-specific T-cells;
contacting a biological sample suspected of having HCMV-specific T-cells with one or more proteins or peptides for detection; and
detecting an amount or a relative amount of, and/or the activity of, and/or the state of antigen-specific T-cells in the biological sample, wherein the one or more proteins or peptides for detection comprise one or more amino acid sequences as claimed in claim 158.

171. A method of detecting a cytomegalovirus infection or exposure in a subject, the method comprising:
contacting a biological sample from a subject with the composition of claim 158; and
determining if the composition elicits an immune response from the contacted cells, wherein the presence of an immune response indicates that the subject has been exposed to or infected with cytomegalovirus.

172. The method of claim 171, wherein the response comprises inducing, increasing, promoting or stimulating anti-cytomegalovirus activity of T cells selected from CD8+ or CD4+ T cells.

173. A kit for the detection of cytomegalovirus or an immune response to cytomegalovirus in a subject comprising:
one or more T cells that specifically detect the presence of:
one or more amino acid sequences selected from the sequences of claim 158; or
a fusion protein of claim 158; or
a pool of 2 or more or more peptides of claim 158.

174. A method of stimulating, inducing, promoting, increasing, or enhancing an immune response against a cytomegalovirus in a subject, comprising administering to the subject the composition of claim 158, in an amount sufficient to stimulate, induce, promote, increase, or enhance an immune response against the cytomegalovirus in the subject.

175. The method of claim 174, wherein the method stimulates, induces, promotes, increases, or enhances an immune response against HCMV in the subject.

176. A method of stimulating, inducing, promoting, increasing, or enhancing an immune response against HCMV in a subject, comprising:
administering to the subject an amount of a protein or peptide or a polynucleotide that expresses the protein or peptide comprising an amino acid sequence selected from the group consisting a HCMV Glycoprotein B, a 65 kDa lower matrix phosphoprotein, a HCMVUL83, a phosphorylated matrix protein (pp65), a tegument protein pp65, a 55 kDa immediate-early protein 1, a regulatory protein IE1, UL123, IE1, a 45 kDa immediate-early protein 2, a single-stranded DNA-binding protein, an envelope glycoprotein H, a glycoprotein H precursor, a major capsid protein, a HCMV UL75 protein or peptide, and variants, homologues, derivatives, or subsequences thereof,
wherein the protein or peptide comprises at least two peptides selected from the amino acid sequences of claim 158,
wherein the amount administered is sufficient to prevent, stimulate, induce, promote, increase, immunize against, or enhance an immune response against HCMV in the subject.

177. A peptide or peptides that are immunoprevalent or immunodominant in a virus prepared by a method consisting essentially of:
obtaining an amino acid sequence of the virus;
determining one or more sets of overlapping peptides spanning one or more virus antigen using unbiased selection;
synthesizing one or more pools of virus peptides comprising the one or more sets of overlapping peptides;
combining the one or more pools of virus peptides with Class I major histocompatibility proteins (MHC), Class II MHC, or both Class I and Class II MHC to form peptide-MHC complexes;
contacting the peptide-MHC complexes with T cells from subjects exposed to the virus; and
determining which pools triggered cytokine release by the T cells; and
deconvoluting from the pool of peptides that elicited cytokine release by the T cells, which peptide or peptides are immunoprevalent or immunodominant in the pool.


SEQUENCE LISTING (439 sequences)

SEQ ID NO: 1 - protein, 15 aa, Human betaherpesvirus 5
NGIRWQYQELQYLVE

SEQ ID NO: 2 - protein, 15 aa, Human betaherpesvirus 5
RYNALTVRSRDSLLL

SEQ ID NO: 3 - protein, 15 aa, Human betaherpesvirus 5
RVRTWFVQRTTLWRR

SEQ ID NO: 4 - protein, 15 aa, Human betaherpesvirus 5
GLWVSSYLVRRPMTI

SEQ ID NO: 5 - protein, 15 aa, Human betaherpesvirus 5
QGATYQLSIVRQAMQ

SEQ ID NO: 6 - protein, 15 aa, Human betaherpesvirus 5
GAGLRQLRQQLTVRW

SEQ ID NO: 7 - protein, 15 aa, Human betaherpesvirus 5
MRTVPVTKLYTSRMV

SEQ ID NO: 8 - protein, 15 aa, Human betaherpesvirus 5
AITLFFFLLALRIPQ

SEQ ID NO: 9 - protein, 15 aa, Human betaherpesvirus 5
ALFTHFVGRPRHCRL

SEQ ID NO: 10 - protein, 15 aa, Human betaherpesvirus 5
MLGIRAMLVMLDYYW

SEQ ID NO: 11 - protein, 15 aa, Human betaherpesvirus 5
PSVRMDFRARRPLRR

SEQ ID NO: 12 - protein, 15 aa, Human betaherpesvirus 5
ARRLWILSLLAVTLT

SEQ ID NO: 13 - protein, 15 aa, Human betaherpesvirus 5
LLAVTLTVALAAPSQ

SEQ ID NO: 14 - protein, 15 aa, Human betaherpesvirus 5
KDRCLVIRRRWRLVR

SEQ ID NO: 15 - protein, 15 aa, Human betaherpesvirus 5
FVAESITEFLNIGLR

SEQ ID NO: 16 - protein, 15 aa, Human betaherpesvirus 5
HENGIYYGTRSMRKL

SEQ ID NO: 17 - protein, 15 aa, Human betaherpesvirus 5
FCRRFFFPDRPDFFL

SEQ ID NO: 18 - protein, 15 aa, Human betaherpesvirus 5
AEDSVFTSTRARSAT

SEQ ID NO: 19 - protein, 15 aa, Human betaherpesvirus 5
KFVLQDFDVQHLRRL

SEQ ID NO: 20 - protein, 15 aa, Human betaherpesvirus 5
IINYYYVAQKKARHM

SEQ ID NO: 21 - protein, 15 aa, Human betaherpesvirus 5
ALALHFLTSRKGVTD

SEQ ID NO: 22 - protein, 15 aa, Human betaherpesvirus 5
LMITHFQRTIRVLRC

SEQ ID NO: 23 - protein, 15 aa, Human betaherpesvirus 5
DFLRVVRQQDAFICT

SEQ ID NO: 24 - protein, 15 aa, Human betaherpesvirus 5
ICVARLQAQPSSRHI

SEQ ID NO: 25 - protein, 15 aa, Human betaherpesvirus 5
GVSSVTLLKIFSQVP

SEQ ID NO: 26 - protein, 15 aa, Human betaherpesvirus 5
VLATLAAVRTRRRSV

SEQ ID NO: 27 - protein, 15 aa, Human betaherpesvirus 5
EAYVRINAGQVLPVV

SEQ ID NO: 28 - protein, 15 aa, Human betaherpesvirus 5
LHCMRYLTSSLVKRY

SEQ ID NO: 29 - protein, 15 aa, Human betaherpesvirus 5
KRYFRPLLRAWSLGL

SEQ ID NO: 30 - protein, 15 aa, Human betaherpesvirus 5
HLLRNIKTAFGMRVL

SEQ ID NO: 31 - protein, 15 aa, Human betaherpesvirus 5
ARNLMEFARVGLRAV

SEQ ID NO: 32 - protein, 15 aa, Human betaherpesvirus 5
TGLVLLLLLLVVRLL

SEQ ID NO: 33 - protein, 15 aa, Human betaherpesvirus 5
MLFRPTISNSIPRCR

SEQ ID NO: 34 - protein, 15 aa, Human betaherpesvirus 5
LRIIRLLRASIRHEY

SEQ ID NO: 35 - protein, 15 aa, Human betaherpesvirus 5
RAHIQKFERLHVRRF

SEQ ID NO: 36 - protein, 15 aa, Human betaherpesvirus 5
SLQFIGLQRRDVVAL

SEQ ID NO: 37 - protein, 15 aa, Human betaherpesvirus 5
RDVVALVNFLRHLTQ

SEQ ID NO: 38 - protein, 15 aa, Human betaherpesvirus 5
RRTVLFNELMLWLGY

SEQ ID NO: 39 - protein, 15 aa, Human betaherpesvirus 5
VNAVNKLVYTGRLIM

SEQ ID NO: 40 - protein, 15 aa, Human betaherpesvirus 5
KELRMCLSFDSNYCR

SEQ ID NO: 41 - protein, 15 aa, Human betaherpesvirus 5
GMKTVAFDLSSPQKS

SEQ ID NO: 42 - protein, 15 aa, Human betaherpesvirus 5
NAIVLITQLLTNRVL

SEQ ID NO: 43 - protein, 15 aa, Human betaherpesvirus 5
STNFLTLTVLPFIVL

SEQ ID NO: 44 - protein, 15 aa, Human betaherpesvirus 5
VLPFIVLSNQWLLPA

SEQ ID NO: 45 - protein, 15 aa, Human betaherpesvirus 5
FATVALIAADRYRVL

SEQ ID NO: 46 - protein, 15 aa, Human betaherpesvirus 5
SYRSTYIILLLTWFA

SEQ ID NO: 47 - protein, 15 aa, Human betaherpesvirus 5
LTLRRTIGTLSRLVP

SEQ ID NO: 48 - protein, 15 aa, Human betaherpesvirus 5
RRRMVSVTLFSPYSV

SEQ ID NO: 49 - protein, 15 aa, Human betaherpesvirus 5
GRLMEVRQRNGRLRR

SEQ ID NO: 50 - protein, 15 aa, Human betaherpesvirus 5
WPERCFIQLRSRSAL

SEQ ID NO: 51 - protein, 15 aa, Human betaherpesvirus 5
GPGFMRYQLIVLIGQ

SEQ ID NO: 52 - protein, 15 aa, Human betaherpesvirus 5
IQTMELMIRTVPRIT

SEQ ID NO: 53 - protein, 15 aa, Human betaherpesvirus 5
EFLVRQYVLVDTFGV

SEQ ID NO: 54 - protein, 15 aa, Human betaherpesvirus 5
RREAIVRLEKTPTCQ

SEQ ID NO: 55 - protein, 15 aa, Human betaherpesvirus 5
RRRFKVCDVGRRHII

SEQ ID NO: 56 - protein, 15 aa, Human betaherpesvirus 5
RHRFLWQRRRRARLL

SEQ ID NO: 57 - protein, 15 aa, Human betaherpesvirus 5
GSFSSFYSQIARSLG

SEQ ID NO: 58 - protein, 15 aa, Human betaherpesvirus 5
FLKKMLLCALKGRAS

SEQ ID NO: 59 - protein, 15 aa, Human betaherpesvirus 5
MPVQRLTVNVARCVF

SEQ ID NO: 60 - protein, 15 aa, Human betaherpesvirus 5
KFIFELYRLPRLSIA

SEQ ID NO: 61 - protein, 15 aa, Human betaherpesvirus 5
ASKIKMLETRVTLAL

SEQ ID NO: 62 - protein, 15 aa, Human betaherpesvirus 5
ATMLSKYTRMSSLFN

SEQ ID NO: 63 - protein, 15 aa, Human betaherpesvirus 5
AFKLDLLRMIAVSRT

SEQ ID NO: 64 - protein, 15 aa, Human betaherpesvirus 5
MLFFQRYAPAFVTGY

SEQ ID NO: 65 - protein, 15 aa, Human betaherpesvirus 5
DLKYILTRLEYLYKV

SEQ ID NO: 66 - protein, 15 aa, Human betaherpesvirus 5
DPSYVREHGVPIHAD

SEQ ID NO: 67 - protein, 15 aa, Human betaherpesvirus 5
TDLIRFERNIVCTSM

SEQ ID NO: 68 - protein, 15 aa, Human betaherpesvirus 5
EGIMVVYKRNIVAHT

SEQ ID NO: 69 - protein, 15 aa, Human betaherpesvirus 5
HTFKVRVYQKVLTFR

SEQ ID NO: 70 - protein, 15 aa, Human betaherpesvirus 5
YQKVLTFRRSYAYIH

SEQ ID NO: 71 - protein, 15 aa, Human betaherpesvirus 5
RRSYAYIHTTYLLGS

SEQ ID NO: 72 - protein, 15 aa, Human betaherpesvirus 5
QLMPDDYSNTHSTRY

SEQ ID NO: 73 - protein, 15 aa, Human betaherpesvirus 5
NLNCMVTITTARSKY

SEQ ID NO: 74 - protein, 15 aa, Human betaherpesvirus 5
NADKFFIFPNYTIVS

SEQ ID NO: 75 - protein, 15 aa, Human betaherpesvirus 5
GLVVFWQGIKQKSLV

SEQ ID NO: 76 - protein, 15 aa, Human betaherpesvirus 5
QLQFTYDTLRGYINR

SEQ ID NO: 77 - protein, 15 aa, Human betaherpesvirus 5
LRGYINRALAQIAEA

SEQ ID NO: 78 - protein, 15 aa, Human betaherpesvirus 5
KELSKINPSAILSAI

SEQ ID NO: 79 - protein, 15 aa, Human betaherpesvirus 5
AILSAIYNKPIAARF

SEQ ID NO: 80 - protein, 15 aa, Human betaherpesvirus 5
ASCVTINQTSVKVLR

SEQ ID NO: 81 - protein, 15 aa, Human betaherpesvirus 5
YLFKRMIDLSSISTV

SEQ ID NO: 82 - protein, 15 aa, Human betaherpesvirus 5
EQAYQMLLALARLDA

SEQ ID NO: 83 - protein, 15 aa, Human betaherpesvirus 5
LLDRLRHRKNGYRHL

SEQ ID NO: 84 - protein, 15 aa, Human betaherpesvirus 5
QILWTDGLARRTRDR

SEQ ID NO: 85 - protein, 15 aa, Human betaherpesvirus 5
RVGITIQQLNVYHQL

SEQ ID NO: 86 - protein, 15 aa, Human betaherpesvirus 5
TMRSVFEMQRIRHGA

SEQ ID NO: 87 - protein, 15 aa, Human betaherpesvirus 5
NIFLVGFYLLVPYLG

SEQ ID NO: 88 - protein, 15 aa, Human betaherpesvirus 5
SLLILVVLLLIYRCC

SEQ ID NO: 89 - protein, 15 aa, Human betaherpesvirus 5
LSYMKYHHLHGLPVN

SEQ ID NO: 90 - protein, 15 aa, Human betaherpesvirus 5
VELCLGAGAGHVVVV

SEQ ID NO: 91 - protein, 15 aa, Human betaherpesvirus 5
RSSWRASCVEVPKKP

SEQ ID NO: 92 - protein, 15 aa, Human betaherpesvirus 5
MQKYFSLDNFLHDYV

SEQ ID NO: 93 - protein, 15 aa, Human betaherpesvirus 5
QTIYFLGLTALLLRY

SEQ ID NO: 94 - protein, 15 aa, Human betaherpesvirus 5
SFYLVNAMSRNLFRV

SEQ ID NO: 95 - protein, 15 aa, Human betaherpesvirus 5
TMRKLKRKQALVKEQ

SEQ ID NO: 96 - protein, 15 aa, Human betaherpesvirus 5
TAVSEFMKNTHVLIR

SEQ ID NO: 97 - protein, 15 aa, Human betaherpesvirus 5
WREDVLMDRVRKRYL

SEQ ID NO: 98 - protein, 15 aa, Human betaherpesvirus 5
IKMWFLLGAPMIAVL

SEQ ID NO: 99 - protein, 15 aa, Human betaherpesvirus 5
LFIIAFFSREPTKDL

SEQ ID NO: 100 - protein, 15 aa, Human betaherpesvirus 5
PKSFTLTRIHPEYIV

SEQ ID NO: 101 - protein, 15 aa, Human betaherpesvirus 5
PEYIVQIQNAFETNQ

SEQ ID NO: 102 - protein, 15 aa, Human betaherpesvirus 5
GALTLVIPSWHVFAS

SEQ ID NO: 103 - protein, 15 aa, Human betaherpesvirus 5
CRSATSLVGNTNADV

SEQ ID NO: 104 - protein, 15 aa, Human betaherpesvirus 5
SSCAHTTCRSATSLV

SEQ ID NO: 105 - protein, 15 aa, Human betaherpesvirus 5
SWLGQMLRPVGLCTL

SEQ ID NO: 106 - protein, 15 aa, Human betaherpesvirus 5
QTGIHVRVSQPSLIL

SEQ ID NO: 107 - protein, 15 aa, Human betaherpesvirus 5
MSIYVYALPLKMLNI

SEQ ID NO: 108 - protein, 15 aa, Human betaherpesvirus 5
PLKMLNIPSINVHHY

SEQ ID NO: 109 - protein, 15 aa, Human betaherpesvirus 5
ATKMQVIGDQYVKVY

SEQ ID NO: 110 - protein, 15 aa, Human betaherpesvirus 5
PKNMIIKPGKISHIM

SEQ ID NO: 111 - protein, 15 aa, Human betaherpesvirus 5
PGKISHIMLDVAFTS

SEQ ID NO: 112 - protein, 15 aa, Human betaherpesvirus 5
MNGQQIFLEVQAIRE

SEQ ID NO: 113 - protein, 15 aa, Human betaherpesvirus 5
ELRQYDPVAALFFFD

SEQ ID NO: 114 - protein, 15 aa, Human betaherpesvirus 5
GILARNLVPMVATVQ

SEQ ID NO: 115 - protein, 15 aa, Human betaherpesvirus 5
ALFFFDIDLLLQRGP

SEQ ID NO: 116 - protein, 15 aa, Human betaherpesvirus 5
RVTGLVFSVVFSVSL

SEQ ID NO: 117 - protein, 15 aa, Human betaherpesvirus 5
LTWCVIADRQPRFSV

SEQ ID NO: 118 - protein, 15 aa, Human betaherpesvirus 5
RPKRRVVAPFRVAAA

SEQ ID NO: 119 - protein, 15 aa, Human betaherpesvirus 5
APFRVAAAGETPLGR

SEQ ID NO: 120 - protein, 15 aa, Human betaherpesvirus 5
IPQRLHLIKHYQLGL

SEQ ID NO: 121 - protein, 15 aa, Human betaherpesvirus 5
IVPMPLALEINQRLL

SEQ ID NO: 122 - protein, 15 aa, Human betaherpesvirus 5
LASELTMTYVRKLAL

SEQ ID NO: 123 - protein, 15 aa, Human betaherpesvirus 5
HSILADFNSYKAHLT

SEQ ID NO: 124 - protein, 15 aa, Human betaherpesvirus 5
FHELRTWEIMEHMRL

SEQ ID NO: 125 - protein, 15 aa, Human betaherpesvirus 5
PQLLFHYRNLVAVLR

SEQ ID NO: 126 - protein, 15 aa, Human betaherpesvirus 5
RNLVAVLRLVTRISA

SEQ ID NO: 127 - protein, 15 aa, Human betaherpesvirus 5
LFLAVQFVGEHVKVL

SEQ ID NO: 128 - protein, 15 aa, Human betaherpesvirus 5
VRVQDLFRVFPMNVY

SEQ ID NO: 129 - protein, 15 aa, Human betaherpesvirus 5
LGYNSKFYSPCAQYF

SEQ ID NO: 130 - protein, 15 aa, Human betaherpesvirus 5
TQEALPILSTTTLAL

SEQ ID NO: 131 - protein, 15 aa, Human betaherpesvirus 5
PFTVLRLSYAYRIFA

SEQ ID NO: 132 - protein, 15 aa, Human betaherpesvirus 5
AREFLLSHDAALFRA

SEQ ID NO: 133 - protein, 15 aa, Human betaherpesvirus 5
MLIQQYVLSQYYIKK

SEQ ID NO: 134 - protein, 15 aa, Human betaherpesvirus 5
RLGTAATQIQKQTLY

SEQ ID NO: 135 - protein, 15 aa, Human betaherpesvirus 5
KTQIFNKLFTNRISV

SEQ ID NO: 136 - protein, 15 aa, Human betaherpesvirus 5
VRSLAVDAQHAAKRV

SEQ ID NO: 137 - protein, 15 aa, Human betaherpesvirus 5
LEERDEWVRSLAVDA

SEQ ID NO: 138 - protein, 15 aa, Human betaherpesvirus 5
AAITVVPVITQSRLA

SEQ ID NO: 139 - protein, 15 aa, Human betaherpesvirus 5
PWYPITQARTLELTP

SEQ ID NO: 140 - protein, 15 aa, Human betaherpesvirus 5
MSTKRSTVPWYPITQ

SEQ ID NO: 141 - protein, 15 aa, Human betaherpesvirus 5
LRVTFHRVKPTLQRE

SEQ ID NO: 142 - protein, 15 aa, Human betaherpesvirus 5
SGRVILWTTLRLCIL

SEQ ID NO: 143 - protein, 15 aa, Human betaherpesvirus 5
VVRKYWTFTNPNRIL

SEQ ID NO: 144 - protein, 15 aa, Human betaherpesvirus 5
TFDVRQFVFDNARLV

SEQ ID NO: 145 - protein, 15 aa, Human betaherpesvirus 5
VRGGIVFNKSVSSVV

SEQ ID NO: 146 - protein, 15 aa, Human betaherpesvirus 5
GNLQVTYVRHYLKNH

SEQ ID NO: 147 - protein, 15 aa, Human betaherpesvirus 5
AVAFLNYSSSSSAVS

SEQ ID NO: 148 - protein, 15 aa, Human betaherpesvirus 5
AGLMMMRRMRRAPAE

SEQ ID NO: 149 - protein, 15 aa, Human betaherpesvirus 5
CDLPLVSSRLLPETS

SEQ ID NO: 150 - protein, 15 aa, Human betaherpesvirus 5
CEIKPYVVNPVVATA

SEQ ID NO: 151 - protein, 15 aa, Human betaherpesvirus 5
DPLLRLSQVAGSGRR

SEQ ID NO: 152 - protein, 15 aa, Human betaherpesvirus 5
LPLCSTARLRLAPRR

SEQ ID NO: 153 - protein, 15 aa, Human betaherpesvirus 5
RATGNFRSTSLYAAV

SEQ ID NO: 154 - protein, 15 aa, Human betaherpesvirus 5
RCCTLRFRRRCRARC

SEQ ID NO: 155 - protein, 15 aa, Human betaherpesvirus 5
MSATRHHRCCTLRFR

SEQ ID NO: 156 - protein, 15 aa, Human betaherpesvirus 5
RVFCLSADWIRFLSL

SEQ ID NO: 157 - protein, 15 aa, Human betaherpesvirus 5
HLGWQTLSNHVIRRL

SEQ ID NO: 158 - protein, 15 aa, Human betaherpesvirus 5
TVVRLHVQIAGRSFT

SEQ ID NO: 159 - protein, 15 aa, Human betaherpesvirus 5
SCTHPYVISLVTPLT

SEQ ID NO: 160 - protein, 15 aa, Human betaherpesvirus 5
ISLVTPLTINATLRL

SEQ ID NO: 161 - protein, 15 aa, Human betaherpesvirus 5
CRVDADLGLLYAVCL

SEQ ID NO: 162 - protein, 15 aa, Human betaherpesvirus 5
VCLILSFSIVTAALW

SEQ ID NO: 163 - protein, 15 aa, Human betaherpesvirus 5
MFFLAIRDHDTAGGI

SEQ ID NO: 164 - protein, 15 aa, Human betaherpesvirus 5
LQTMLRKEVNSQLSL

SEQ ID NO: 165 - protein, 15 aa, Human betaherpesvirus 5
LVKQIKVRVDMVRHR

SEQ ID NO: 166 - protein, 15 aa, Human betaherpesvirus 5
RVDMVRHRIKEHMLK

SEQ ID NO: 167 - protein, 15 aa, Human betaherpesvirus 5
LRRKMMYMCYRNIEF

SEQ ID NO: 168 - protein, 15 aa, Human betaherpesvirus 5
CSPDEIMSYAQKIFK

SEQ ID NO: 169 - protein, 15 aa, Human betaherpesvirus 5
EERDKVLTHIDHIFM

SEQ ID NO: 170 - protein, 15 aa, Human betaherpesvirus 5
VLCCYVLEETSVMLA

SEQ ID NO: 171 - protein, 15 aa, Human betaherpesvirus 5
ITKPEVISVMKRRIE

SEQ ID NO: 172 - protein, 15 aa, Human betaherpesvirus 5
FAQYILGADPLRVCS

SEQ ID NO: 173 - protein, 15 aa, Human betaherpesvirus 5
EAIVAYTLATAGASS

SEQ ID NO: 174 - protein, 15 aa, Human betaherpesvirus 5
TTRPFKVIIKPPVPP

SEQ ID NO: 175 - protein, 15 aa, Human betaherpesvirus 5
NKGIQIIYTRNHEVK

SEQ ID NO: 176 - protein, 15 aa, Human betaherpesvirus 5
LGSMCNLALSTPFLM

SEQ ID NO: 177 - protein, 15 aa, Human betaherpesvirus 5
STPFLMEHTMPVTHP

SEQ ID NO: 178 - protein, 15 aa, Human betaherpesvirus 5
YRNMIIHAATPVDLL

SEQ ID NO: 179 - protein, 15 aa, Human betaherpesvirus 5
VMVRIFSTNQGGFML

SEQ ID NO: 180 - protein, 15 aa, Human betaherpesvirus 5
VVVGIVLCLSLASTV

SEQ ID NO: 181 - protein, 15 aa, Human betaherpesvirus 5
SPVAAELPHPSPAPM

SEQ ID NO: 182 - protein, 15 aa, Human betaherpesvirus 5
SYLAVHLRISHRYYH

SEQ ID NO: 183 - protein, 15 aa, Human betaherpesvirus 5
IAITMVMRFWQYING

SEQ ID NO: 184 - protein, 15 aa, Human betaherpesvirus 5
TALWLLLGHSRVPRV

SEQ ID NO: 185 - protein, 15 aa, Human betaherpesvirus 5
AEIRGIVTTMTHSLT

SEQ ID NO: 186 - protein, 15 aa, Human betaherpesvirus 5
NPLYLEADGRIRCGK

SEQ ID NO: 187 - protein, 15 aa, Human betaherpesvirus 5
LHRRAAVSGRRSLLQ

SEQ ID NO: 188 - protein, 15 aa, Human betaherpesvirus 5
MLRLLFTLVLLALYG

SEQ ID NO: 189 - protein, 15 aa, Human betaherpesvirus 5
HVRLLSYRGDPLVFK

SEQ ID NO: 190 - protein, 15 aa, Human betaherpesvirus 5
VVRFALYLETLSRIV

SEQ ID NO: 191 - protein, 15 aa, Human betaherpesvirus 5
FYMNWTLRRSQTHYL

SEQ ID NO: 192 - protein, 15 aa, Human betaherpesvirus 5
QVEILKPRGVRHRAI

SEQ ID NO: 193 - protein, 15 aa, Human betaherpesvirus 5
FCVYRYNARLTRGYV

SEQ ID NO: 194 - protein, 15 aa, Human betaherpesvirus 5
TRGYVRYTLSPKARL

SEQ ID NO: 195 - protein, 15 aa, Human betaherpesvirus 5
SLDRFIVQYLNTLLI

SEQ ID NO: 196 - protein, 15 aa, Human betaherpesvirus 5
PTWSTTVNAHNSFLH

SEQ ID NO: 197 - protein, 15 aa, Human betaherpesvirus 5
DRLSTLAATMCMFDY

SEQ ID NO: 198 - protein, 15 aa, Human betaherpesvirus 5
LFYRAVALGTLSALV

SEQ ID NO: 199 - protein, 15 aa, Human betaherpesvirus 5
SSIFTSTHRGVIVAP

SEQ ID NO: 200 - protein, 15 aa, Human betaherpesvirus 5
LSVRYLSLTAYMLLA

SEQ ID NO: 201 - protein, 15 aa, Human betaherpesvirus 5
TAYKAFLWKYAKKLN

SEQ ID NO: 202 - protein, 15 aa, Human betaherpesvirus 5
WKYAKKLNYHYFRLR

SEQ ID NO: 203 - protein, 15 aa, Human betaherpesvirus 5
VYLWYVRRQLVAFCL

SEQ ID NO: 204 - protein, 15 aa, Human betaherpesvirus 5
FPSARDIPKQLPEQP

SEQ ID NO: 205 - protein, 15 aa, Human betaherpesvirus 5
VVAYVILERLWLAAR

SEQ ID NO: 206 - protein, 15 aa, Human betaherpesvirus 5
IRRWWISVAIVIFIG

SEQ ID NO: 207 - protein, 15 aa, Human betaherpesvirus 5
RWQFAVCAASKTATR

SEQ ID NO: 208 - protein, 15 aa, Human betaherpesvirus 5
PQRLLLTALAIWQRT

SEQ ID NO: 209 - protein, 15 aa, Human betaherpesvirus 5
PWWRRLRVKRPKFPS

SEQ ID NO: 210 - protein, 15 aa, Human betaherpesvirus 5
LWYLGDYGAILKIYF

SEQ ID NO: 211 - protein, 15 aa, Human betaherpesvirus 5
LFCGACVITRSLLLI

SEQ ID NO: 212 - protein, 15 aa, Human betaherpesvirus 5
MNLVMLILALWAPVA

SEQ ID NO: 213 - protein, 15 aa, Human betaherpesvirus 5
VSEYRVEYSEARCVL

SEQ ID NO: 214 - protein, 15 aa, Human betaherpesvirus 5
MLVVTVFDTTRLFEI

SEQ ID NO: 215 - protein, 15 aa, Human betaherpesvirus 5
VCAFCWLVLPHRLEQ

SEQ ID NO: 216 - protein, 15 aa, Human betaherpesvirus 5
VSVLYFMPSEPGSAH

SEQ ID NO: 217 - protein, 15 aa, Human betaherpesvirus 5
VFQKTLSMLQGLYLR

SEQ ID NO: 218 - protein, 15 aa, Human betaherpesvirus 5
GLYLRQYDPPALRTY

SEQ ID NO: 219 - protein, 15 aa, Human betaherpesvirus 5
WFLVMREQAAIPQIY

SEQ ID NO: 220 - protein, 15 aa, Human betaherpesvirus 5
QIYARSLAADYLCCD

SEQ ID NO: 221 - protein, 15 aa, Human betaherpesvirus 5
DFRDLLNFIRQRLCC

SEQ ID NO: 222 - protein, 15 aa, Human betaherpesvirus 5
PSQEILLLCARHLDE

SEQ ID NO: 223 - protein, 15 aa, Human betaherpesvirus 5
TDCWPFEVAPAARLA

SEQ ID NO: 224 - protein, 15 aa, Human betaherpesvirus 5
LFRAGLMKVYVRRRY

SEQ ID NO: 225 - protein, 15 aa, Human betaherpesvirus 5
VVFMGRFSRVYAYDT

SEQ ID NO: 226 - protein, 15 aa, Human betaherpesvirus 5
EKYMVLVSHNLDELA

SEQ ID NO: 227 - protein, 15 aa, Human betaherpesvirus 5
PRLHCLVTTRSSTRE

SEQ ID NO: 228 - protein, 15 aa, Human betaherpesvirus 5
LRYKWLIRKDRFIVR

SEQ ID NO: 229 - protein, 15 aa, Human betaherpesvirus 5
TNIMLQVSNVTNHTL

SEQ ID NO: 230 - protein, 15 aa, Human betaherpesvirus 5
IVVGLPFFLEYAKHH

SEQ ID NO: 231 - protein, 15 aa, Human betaherpesvirus 5
YNRMVRFIINYVGKW

SEQ ID NO: 232 - protein, 15 aa, Human betaherpesvirus 5
ITFCLYVGQFLAYVR

SEQ ID NO: 233 - protein, 15 aa, Human betaherpesvirus 5
HDPLGLTRFIMRQLM

SEQ ID NO: 234 - protein, 15 aa, Human betaherpesvirus 5
FIMRQLMMYPLVLPF

SEQ ID NO: 235 - protein, 15 aa, Human betaherpesvirus 5
GLVYRELHDFYGYLQ

SEQ ID NO: 236 - protein, 17 aa, Human betaherpesvirus 5
HINSHSQCYSSYSRVIA

SEQ ID NO: 237 - protein, 15 aa, Human betaherpesvirus 5
SRVIAGTVFVAYHRD

SEQ ID NO: 238 - protein, 15 aa, Human betaherpesvirus 5
CMVTITTARSKYPYH

SEQ ID NO: 239 - protein, 15 aa, Human betaherpesvirus 5
VFETTGGLVVFWQGI

SEQ ID NO: 240 - protein, 20 aa, Human betaherpesvirus 5
MQLIPDDYSNTHSTRYVTVK

SEQ ID NO: 241 - protein, 14 aa, Human betaherpesvirus 5
LPLKMLNIPSINVH

SEQ ID NO: 242 - protein, 16 aa, Human betaherpesvirus 5
PQYSEHPTFTSQYRIQ

SEQ ID NO: 243 - protein, 16 aa, Human betaherpesvirus 5
FTSQYRIQGKLEYRHT

SEQ ID NO: 244 - protein, 16 aa, Human betaherpesvirus 5
PPWQAGILARNLVPMV

SEQ ID NO: 245 - protein, 16 aa, Human betaherpesvirus 5
KYQEFFWDANDIYRIF

SEQ ID NO: 246 - protein, 15 aa, Human betaherpesvirus 5
GPISGHVLKAVFSRG

SEQ ID NO: 247 - protein, 15 aa, Human betaherpesvirus 5
LLQTGIHVRVSQPSL

SEQ ID NO: 248 - protein, 15 aa, Human betaherpesvirus 5
IYVYALPLKMLNIPS

SEQ ID NO: 249 - protein, 15 aa, Human betaherpesvirus 5
LPLKMLNIPSINVHH

SEQ ID NO: 250 - protein, 15 aa, Human betaherpesvirus 5
KDVALRHVVCAHELV

SEQ ID NO: 251 - protein, 15 aa, Human betaherpesvirus 5
RHVVCAHELVCSMEN

SEQ ID NO: 252 - protein, 15 aa, Human betaherpesvirus 5
CSMENTRATKMQVIG

SEQ ID NO: 253 - protein, 15 aa, Human betaherpesvirus 5
TRATKMQVIGDQYVK

SEQ ID NO: 254 - protein, 15 aa, Human betaherpesvirus 5
MQVIGDQYVKVYLES

SEQ ID NO: 255 - protein, 15 aa, Human betaherpesvirus 5
VYLESFCEDVPSGKL

SEQ ID NO: 256 - protein, 15 aa, Human betaherpesvirus 5
FCEDVPSGKLFMHVT

SEQ ID NO: 257 - protein, 15 aa, Human betaherpesvirus 5
LGSDVEEDLTMTRNP

SEQ ID NO: 258 - protein, 15 aa, Human betaherpesvirus 5
EEDLTMTRNPQPFMR

SEQ ID NO: 259 - protein, 15 aa, Human betaherpesvirus 5
QPFMRPHERNGFTVL

SEQ ID NO: 260 - protein, 15 aa, Human betaherpesvirus 5
KISHIMLDVAFTSHE

SEQ ID NO: 261 - protein, 15 aa, Human betaherpesvirus 5
MLDVAFTSHEHFGLL

SEQ ID NO: 262 - protein, 15 aa, Human betaherpesvirus 5
FTSHEHFGLLCPKSI

SEQ ID NO: 263 - protein, 15 aa, Human betaherpesvirus 5
PQYSEHPTFTSQYRI

SEQ ID NO: 264 - protein, 15 aa, Human betaherpesvirus 5
SQYRIQGKLEYRHTW

SEQ ID NO: 265 - protein, 15 aa, Human betaherpesvirus 5
YRHTWDRHDEGAAQG

SEQ ID NO: 266 - protein, 15 aa, Human betaherpesvirus 5
IHNPAVFTWPPWQAG

SEQ ID NO: 267 - protein, 15 aa, Human betaherpesvirus 5
PWQAGILARNLVPMV

SEQ ID NO: 268 - protein, 15 aa, Human betaherpesvirus 5
ATVQGQNLKYQEFFW

SEQ ID NO: 269 - protein, 15 aa, Human betaherpesvirus 5
QNLKYQEFFWDANDI

SEQ ID NO: 270 - protein, 15 aa, Human betaherpesvirus 5
QEFFWDANDIYRIFA

SEQ ID NO: 271 - protein, 15 aa, Human betaherpesvirus 5
ELEGVWQPAAQPKRR

SEQ ID NO: 272 - protein, 15 aa, Human betaherpesvirus 5
IFLEVQAIRETVELR

SEQ ID NO: 273 - protein, 15 aa, Human betaherpesvirus 5
PPWQAGILARNLVPM

SEQ ID NO: 274 - protein, 15 aa, Human betaherpesvirus 5
DVPSGKLFMHVTLGS

SEQ ID NO: 275 - protein, 15 aa, Human betaherpesvirus 5
KLFMHVTLGSDVEED

SEQ ID NO: 276 - protein, 15 aa, Human betaherpesvirus 5
DVEEDLTMTRNPQPF

SEQ ID NO: 277 - protein, 15 aa, Human betaherpesvirus 5
VAFTSHEHFGLLCPK

SEQ ID NO: 278 - protein, 15 aa, Human betaherpesvirus 5
SEHPTFTSQYRIQGK

SEQ ID NO: 279 - protein, 15 aa, Human betaherpesvirus 5
LEYRHTWDRHDEGAA

SEQ ID NO: 280 - protein, 15 aa, Human betaherpesvirus 5
KVYLESFCEDVPSGK

SEQ ID NO: 281 - protein, 15 aa, Human betaherpesvirus 5
TLGSDVEEDLTMTRN

SEQ ID NO: 282 - protein, 15 aa, Human betaherpesvirus 5
ASTSAGRKRKSASSA

SEQ ID NO: 283 - protein, 15 aa, Human betaherpesvirus 5
ACTSGVMTRGRLKAE

SEQ ID NO: 284 - protein, 15 aa, Human betaherpesvirus 5
AGILARNLVPMVATV

SEQ ID NO: 285 - protein, 15 aa, Human betaherpesvirus 5
EPDVYYTSAFVFPTK

SEQ ID NO: 286 - protein, 15 aa, Human betaherpesvirus 5
QVIGDQYVKVYLESF

SEQ ID NO: 287 - protein, 15 aa, Human betaherpesvirus 5
FFWDANDIYRIFAEL

SEQ ID NO: 288 - protein, 15 aa, Human betaherpesvirus 5
LVSQYTPDSTPCHRG

SEQ ID NO: 289 - protein, 14 aa, Human betaherpesvirus 5
SHIMLDVAFTSHEH

SEQ ID NO: 290 - protein, 16 aa, Human betaherpesvirus 5
DEDSDNEIHNPAVFTW

SEQ ID NO: 291 - protein, 13 aa, Human betaherpesvirus 5
SQYTPDSTPCHRG

SEQ ID NO: 292 - protein, 13 aa, Human betaherpesvirus 5
KPGKISHIMLDVA

SEQ ID NO: 293 - protein, 13 aa, Human betaherpesvirus 5
PTFTSQYRIQGKL

SEQ ID NO: 294 - protein, 20 aa, Human betaherpesvirus 5
DTPVLPHETRLLQTGIHVRV

SEQ ID NO: 295 - protein, 20 aa, Human betaherpesvirus 5
INVHHYPSAAERKHRHLPVA

SEQ ID NO: 296 - protein, 15 aa, Human betaherpesvirus 5
LLQRGPQYSEHPTFT

SEQ ID NO: 297 - protein, 19 aa, Human betaherpesvirus 5
ALFFFDIDLLLQRGPQYSE

SEQ ID NO: 298 - protein, 20 aa, Human betaherpesvirus 5
DQYVKVYLESFCEDVPSGKL

SEQ ID NO: 299 - protein, 20 aa, Human betaherpesvirus 5
MTRNPQPFMRPHERNGFTVL

SEQ ID NO: 300 - protein, 20 aa, Human betaherpesvirus 5
MISVLGPISGHVLKAVFSRG

SEQ ID NO: 301 - protein, 20 aa, Human betaherpesvirus 5
ASGKQMWQARLTVSGLAWTR

SEQ ID NO: 302 - protein, 20 aa, Human betaherpesvirus 5
LPLKMLNIPSINVHHYPSAA

SEQ ID NO: 303 - protein, 20 aa, Human betaherpesvirus 5
PHETRLLQTGIHVRVSQPSL

SEQ ID NO: 304 - protein, 20 aa, Human betaherpesvirus 5
IYVYALPLKMLNIPSINVHH

SEQ ID NO: 305 - protein, 20 aa, Human betaherpesvirus 5
QYDPVAALFFFDIDLLLQRG

SEQ ID NO: 306 - protein, 16 aa, Human betaherpesvirus 5
RQYDPVAALFFFDIDL

SEQ ID NO: 307 - protein, 15 aa, Human betaherpesvirus 5
HETRLLQTGIHVRVS

SEQ ID NO: 308 - protein, 15 aa, Human betaherpesvirus 5
VYALPLKMLNIPSIN

SEQ ID NO: 309 - protein, 15 aa, Human betaherpesvirus 5
VALRHVVCAHELVCS

SEQ ID NO: 310 - protein, 15 aa, Human betaherpesvirus 5
HIMLDVAFTSHEHFG

SEQ ID NO: 311 - protein, 15 aa, Human betaherpesvirus 5
FTSQYRIQGKLEYRH

SEQ ID NO: 312 - protein, 15 aa, Human betaherpesvirus 5
YRIQGKLEYRHTWDR

SEQ ID NO: 313 - protein, 15 aa, Human betaherpesvirus 5
ARNLVPMVATVQGQN

SEQ ID NO: 314 - protein, 15 aa, Human betaherpesvirus 5
ANDIYRIFAELEGVW

SEQ ID NO: 315 - protein, 15 aa, Human betaherpesvirus 5
TRQQNQWKEPDVYYT

SEQ ID NO: 316 - protein, 15 aa, Human betaherpesvirus 5
TERKTPRVTGGGAMA

SEQ ID NO: 317 - protein, 15 aa, Human betaherpesvirus 5
NLKYQEFFWDANDIY

SEQ ID NO: 318 - protein, 15 aa, Human betaherpesvirus 5
TPRVTGGGAMAGAST

SEQ ID NO: 319 - protein, 15 aa, Human betaherpesvirus 5
DQYVKVYLESFCEDV

SEQ ID NO: 320 - protein, 15 aa, Human betaherpesvirus 5
GKISHIMLDVAFTSH

SEQ ID NO: 321 - protein, 15 aa, Human betaherpesvirus 5
EHPTFTSQYRIQGKL

SEQ ID NO: 322 - protein, 15 aa, Human betaherpesvirus 5
GQNLKYQEFFWDAND

SEQ ID NO: 323 - protein, 15 aa, Human betaherpesvirus 5
KYQEFFWDANDIYRI

SEQ ID NO: 324 - protein, 15 aa, Human betaherpesvirus 5
IIKPGKISHIMLDVA

SEQ ID NO: 325 - protein, 20 aa, Human betaherpesvirus 5
TRATKMQVIGDQYVKVYLES

SEQ ID NO: 326 - protein, 20 aa, Human betaherpesvirus 5
KLFMHVTLGSDVEEDLTMTR

SEQ ID NO: 327 - protein, 20 aa, Human betaherpesvirus 5
KPGKISHIMLDVAFTSHEHF

SEQ ID NO: 328 - protein, 20 aa, Human betaherpesvirus 5
LPVADAVIHASGKQMWQARL

SEQ ID NO: 329 - protein, 20 aa, Human betaherpesvirus 5
GSDSDEELVTTERKTPRVTG

SEQ ID NO: 330 - protein, 20 aa, Human betaherpesvirus 5
RHRQDALPGPCIASTPKKHR

SEQ ID NO: 331 - protein, 13 aa, Human betaherpesvirus 5
YQEFFWDANDIYR

SEQ ID NO: 332 - protein, 15 aa, Human betaherpesvirus 5
LAWTRQQNQWKEPDV

SEQ ID NO: 333 - protein, 15 aa, Human betaherpesvirus 5
YQEFFWDANDIYRIF

SEQ ID NO: 334 - protein, 13 aa, Human betaherpesvirus 5
EFFWDANDIYRIF

SEQ ID NO: 335 - protein, 15 aa, Human betaherpesvirus 5
VEEDLTMTRNPQPFM

SEQ ID NO: 336 - protein, 17 aa, Human betaherpesvirus 5
KPGKISHIMLDVAFTSH

SEQ ID NO: 337 - protein, 15 aa, Human betaherpesvirus 5
TSQYRIQGKLEYRHT

SEQ ID NO: 338 - protein, 15 aa, Human betaherpesvirus 5
VYYTSAFVFPTKDVA

SEQ ID NO: 339 - protein, 15 aa, Human betaherpesvirus 5
LRQYDPVAALFFFDI

SEQ ID NO: 340 - protein, 16 aa, Human betaherpesvirus 5
GPQYSEHPTFTSQYRI

SEQ ID NO: 341 - protein, 15 aa, Human betaherpesvirus 5
HPTFTSQYRIQGKLE

SEQ ID NO: 342 - protein, 15 aa, Human betaherpesvirus 5
TRLLQTGIHVRVSQP

SEQ ID NO: 343 - protein, 15 aa, Human betaherpesvirus 5
RNGFTVLCPKNMIIK

SEQ ID NO: 344 - protein, 15 aa, Human betaherpesvirus 5
PISGHVLKAVFSRGD

SEQ ID NO: 345 - protein, 15 aa, Human betaherpesvirus 5
GIHVRVSQPSLILVS

SEQ ID NO: 346 - protein, 15 aa, Human betaherpesvirus 5
IHASGKQMWQARLTV

SEQ ID NO: 347 - protein, 15 aa, Human betaherpesvirus 5
GKQMWQARLTVSGLA

SEQ ID NO: 348 - protein, 15 aa, Human betaherpesvirus 5
ENTRATKMQVIGDQY

SEQ ID NO: 349 - protein, 15 aa, Human betaherpesvirus 5
RPHERNGFTVLCPKN

SEQ ID NO: 350 - protein, 15 aa, Human betaherpesvirus 5
AQGDDDVWTSGSDSD

SEQ ID NO: 351 - protein, 15 aa, Human betaherpesvirus 5
SSATACTSGVMTRGR

SEQ ID NO: 352 - protein, 15 aa, Human betaherpesvirus 5
YRIFAELEGVWQPAA

SEQ ID NO: 353 - protein, 15 aa, Human betaherpesvirus 5
AELEGVWQPAAQPKR

SEQ ID NO: 354 - protein, 15 aa, Human betaherpesvirus 5
AVFSRGDTPVLPHET

SEQ ID NO: 355 - protein, 15 aa, Human betaherpesvirus 5
ALPLKMLNIPSINVH

SEQ ID NO: 356 - protein, 15 aa, Human betaherpesvirus 5
HVLKAVFSRGDTPVL

SEQ ID NO: 357 - protein, 20 aa, Human betaherpesvirus 5
AHELVCSMENTRATKMQVIG

SEQ ID NO: 358 - protein, 20 aa, Human betaherpesvirus 5
FCEDVPSGKLFMHVTLGSDV

SEQ ID NO: 359 - protein, 19 aa, Human betaherpesvirus 5
TLGSDVEEDLTMTRNPQPF

SEQ ID NO: 360 - protein, 18 aa, Human betaherpesvirus 5
LLQTGIHVRVSQPSLILV

SEQ ID NO: 361 - protein, 15 aa, Human betaherpesvirus 5
SICPSQEPMSIYVYA

SEQ ID NO: 362 - protein, 15 aa, Human betaherpesvirus 5
SQEPMSIYVYALPLK

SEQ ID NO: 363 - protein, 15 aa, Human betaherpesvirus 5
LNIPSINVHHYPSAA

SEQ ID NO: 364 - protein, 19 aa, Human betaherpesvirus 5
HDVSKGDDNKLGGALQAKA

SEQ ID NO: 365 - protein, 19 aa, Human betaherpesvirus 5
ALQAKARDKKDELRRKMMY

SEQ ID NO: 366 - protein, 15 aa, Human betaherpesvirus 5
KEHMLKKYTQTEEKF

SEQ ID NO: 367 - protein, 19 aa, Human betaherpesvirus 5
QTEEKFTGAFNMMGGCLQN

SEQ ID NO: 368 - protein, 20 aa, Human betaherpesvirus 5
MGGCLQNALDILDKVHEPFE

SEQ ID NO: 369 - protein, 19 aa, Human betaherpesvirus 5
AIVAYTLATAGVSSSDSLV

SEQ ID NO: 370 - protein, 19 aa, Human betaherpesvirus 5
TMQSMYENYIVPEDKREMW

SEQ ID NO: 371 - protein, 19 aa, Human betaherpesvirus 5
RRKMMYMCYRNIEFFTKNS

SEQ ID NO: 372 - protein, 19 aa, Human betaherpesvirus 5
FFTKNSAFPKTTNGCSQAM

SEQ ID NO: 373 - protein, 19 aa, Human betaherpesvirus 5
CVETMCNEYKVTSDACMMT

SEQ ID NO: 374 - protein, 19 aa, Human betaherpesvirus 5
DACMMTMYGGASLLSEFCR

SEQ ID NO: 375 - protein, 20 aa, Human betaherpesvirus 5
NYIVPEDKREMWMACIKELH

SEQ ID NO: 376 - protein, 20 aa, Human betaherpesvirus 5
VRHRIKEHMLKKYTQTEEKF

SEQ ID NO: 377 - protein, 15 aa, Human betaherpesvirus 5
VRVDMVRHRIKEHML

SEQ ID NO: 378 - protein, 15 aa, Human betaherpesvirus 5
VKQIKVRVDMVRHRI

SEQ ID NO: 379 - protein, 15 aa, Human betaherpesvirus 5
VRHRIKEHMLKKYTQ

SEQ ID NO: 380 - protein, 15 aa, Human betaherpesvirus 5
EQSDEEEEEGAQEER

SEQ ID NO: 381 - protein, 15 aa, Human betaherpesvirus 5
VKSEPVSEIEEVAPE

SEQ ID NO: 382 - protein, 15 aa, Human betaherpesvirus 5
PVSEIEEVAPEEEED

SEQ ID NO: 383 - protein, 15 aa, Human betaherpesvirus 5
LQNALDILDKVHEPF

SEQ ID NO: 384 - protein, 15 aa, Human betaherpesvirus 5
EDKREMWMACIKELH

SEQ ID NO: 385 - protein, 15 aa, Human betaherpesvirus 5
THIDHIFMDILTTCV

SEQ ID NO: 386 - protein, 15 aa, Human betaherpesvirus 5
VLEETSVMLAKRPLI

SEQ ID NO: 387 - protein, 15 aa, Human betaherpesvirus 5
TKPEVISVMKRRIEE

SEQ ID NO: 388 - protein, 15 aa, Human betaherpesvirus 5
RRIEEICMKVFAQYI

SEQ ID NO: 389 - protein, 15 aa, Human betaherpesvirus 5
NIEFFTKNSAFPKTT

SEQ ID NO: 390 - protein, 19 aa, Human betaherpesvirus 5
LTHIDHIFMDILTTCVETM

SEQ ID NO: 391 - protein, 19 aa, Human betaherpesvirus 5
AIVAYTLATAGASSSDSLV

SEQ ID NO: 392 - protein, 20 aa, Human betaherpesvirus 5
VRVDMVRHRIKEHMLKKYTQ

SEQ ID NO: 393 - protein, 14 aa, Human betaherpesvirus 5
DKREMWMACIKELH

SEQ ID NO: 394 - protein, 20 aa, Human betaherpesvirus 5
QSMYENYIVPEDKREMWMAC

SEQ ID NO: 395 - protein, 15 aa, Human betaherpesvirus 5
TRRGRVKIDEVSRMF

SEQ ID NO: 396 - protein, 15 aa, Human betaherpesvirus 5
GDILAQAVNHAGIDS

SEQ ID NO: 397 - protein, 15 aa, Human betaherpesvirus 5
KTTRPFKVIIKPPVP

SEQ ID NO: 398 - protein, 15 aa, Human betaherpesvirus 5
FKVIIKPPVPPAPIM

SEQ ID NO: 399 - protein, 15 aa, Human betaherpesvirus 5
PEPDFTIQYRNKIID

SEQ ID NO: 400 - protein, 15 aa, Human betaherpesvirus 5
PFTIPSMHQVLDEAI

SEQ ID NO: 401 - protein, 15 aa, Human betaherpesvirus 5
LMQKFPKQVMVRIFS

SEQ ID NO: 402 - protein, 15 aa, Human betaherpesvirus 5
VRIFSTNQGGFMLPI

SEQ ID NO: 403 - protein, 15 aa, Human betaherpesvirus 5
PEDLDTLSLAIEAAI

SEQ ID NO: 404 - protein, 15 aa, Human betaherpesvirus 5
TLSLAIEAAIQDLRN

SEQ ID NO: 405 - protein, 15 aa, Human betaherpesvirus 5
SMHQVLDEAIKACKT

SEQ ID NO: 406 - protein, 15 aa, Human betaherpesvirus 5
KGIQIIYTRNHEVKS

SEQ ID NO: 407 - protein, 15 aa, Human betaherpesvirus 5
ALSTPFLMEHTMPVT

SEQ ID NO: 408 - protein, 15 aa, Human betaherpesvirus 5
FLMEHTMPVTHPPEV

SEQ ID NO: 409 - protein, 14 aa, Human betaherpesvirus 5
PYAVAFQPLLAYAY

SEQ ID NO: 410 - protein, 19 aa, Human betaherpesvirus 5
KTQLNRHSYLKDSDFLDAA

SEQ ID NO: 411 - protein, 20 aa, Human betaherpesvirus 5
RQTEKHELLVLVKKAQLNRH

SEQ ID NO: 412 - protein, 18 aa, Human betaherpesvirus 5
LDPHAFHLLLNTYGRPIR

SEQ ID NO: 413 - protein, 19 aa, Human betaherpesvirus 5
KAQLNRHSYLKDSDFLDAA

SEQ ID NO: 414 - protein, 19 aa, Human betaherpesvirus 5
DVLKSGRCQMLDRRTVEMA

SEQ ID NO: 415 - protein, 17 aa, Human betaherpesvirus 5
LDKAFHLLLNTYGRPIR

SEQ ID NO: 416 - protein, 19 aa, Human betaherpesvirus 5
KDQLNRHSYLKDPDFLDAA

SEQ ID NO: 417 - protein, 13 aa, Human betaherpesvirus 5
SYLKDSDFLDAAL

SEQ ID NO: 418 - protein, 19 aa, Human betaherpesvirus 5
RRIPHFYRVRREVPRTVNE

SEQ ID NO: 419 - protein, 19 aa, Human betaherpesvirus 5
MDVNYFKIPNNPRGRASCM

SEQ ID NO: 420 - protein, 15 aa, Human betaherpesvirus 5
MHHLVVHPFLPLATL

SEQ ID NO: 421 - protein, 15 aa, Human betaherpesvirus 5
FLPLATLFLFVVIAD

SEQ ID NO: 422 - protein, 15 aa, Human betaherpesvirus 5
IVERALVSAVILAKM

SEQ ID NO: 423 - protein, 15 aa, Human betaherpesvirus 5
AVILAKMSVRETLVT

SEQ ID NO: 424 - protein, 15 aa, Human betaherpesvirus 5
PVTTAYKFEQPTLTF

SEQ ID NO: 425 - protein, 15 aa, Human betaherpesvirus 5
SRIRKAKLSSPMTTT

SEQ ID NO: 426 - protein, 15 aa, Human betaherpesvirus 5
AVINTFIIFVGGPLN

SEQ ID NO: 427 - protein, 15 aa, Human betaherpesvirus 5
LLTNRVLGYSTPTIY

SEQ ID NO: 428 - protein, 15 aa, Human betaherpesvirus 5
FLSVIYYSSCTVGFA

SEQ ID NO: 429 - protein, 15 aa, Human betaherpesvirus 5
WFAGLIFSMPAAVYT

SEQ ID NO: 430 - protein, 15 aa, Human betaherpesvirus 5
FIADEVYTVLLSWKV

SEQ ID NO: 431 - protein, 15 aa, Human betaherpesvirus 5
WKVLLTLVWGAAPVI

SEQ ID NO: 432 - protein, 15 aa, Human betaherpesvirus 5
MTWFYAFFYSTVQRA

SEQ ID NO: 433 - protein, 15 aa, Human betaherpesvirus 5
RSRTLTFVSVLLISF

SEQ ID NO: 434 - protein, 15 aa, Human betaherpesvirus 5
LLISFVALQTPYVSI

SEQ ID NO: 435 - protein, 15 aa, Human betaherpesvirus 5
YVSIMIFNSYATAAW

SEQ ID NO: 436 - protein, 15 aa, Human betaherpesvirus 5
PILYALLGHDFLQRM

SEQ ID NO: 437 - protein, 15 aa, Human betaherpesvirus 5
RQCFRGQLLDRRAFL

SEQ ID NO: 438 - protein, 15 aa, Human betaherpesvirus 5
DRRAFLRSQQNQRAT

SEQ ID NO: 439 - protein, 14 aa, Human betaherpesvirus 5
SYRSTYIILLTWFA